(19) United States
(12) Patent Application Publication
(10) Pub. No.: US 2010/0267007 A1
(43) Pub. Date: Oct. 21, 2010

(54) SOLID-FLUID COMPOSITION AND USES THEREOF

(75) Inventor: Eran GABBAI, Kfar-MaAs (IL)

Correspondence Address: FOLEY AND LARDNER LLP;SUITE 500, 3000 K STREET NW, WASHINGTON, DC, 20007, US

(73) Assignee: Do-Coop Technologies LTD.

(21) Appl. No.: 12/350,710

(22) Filed: Jan. 8, 2009

Related U.S. Application Data
(62) Division of application No. 10/865,955, filed on Jun. 14, 2004.
Application No. 10/865,955 is a continuation-in-part of application No. PCT/IL02/01004, filed on Dec. 12, 2002.
(60) Provisional application No. 60/545,955, filed on Feb. 20, 2004.

(30) Foreign Application Priority Data
Dec. 12, 2001 (IL) 147049

(51) Int. Cl. C12Q 1/70; C12Q 1/02; G01N 33/53; C12Q 1/68; C12Q 1/42; C12Q 1/34

(52) U.S. Cl. 435/5; 435/29; 436/501; 435/6; 435/21; 435/18; 204/157.15; 977/924; 977/904

(57) ABSTRACT

A nanostructure comprising a core material of a nanometric size surrounded by an envelope of ordered fluid molecules is disclosed. The core material and the envelope of ordered fluid molecules are in a steady physical state. Also disclosed, a liquid composition comprising liquid and the nanostructure.

### CROSS-REFERENCE TO RELATED PATENT APPLICATIONS

This application claims the benefit of U.S. patent application Ser. No. 60/545,955 filed Feb. 20, 2004. This application is also a continuation-in-part of PCT Patent Application No. PCT/IL02/01004 filed Dec. 12, 2002, which claims priority from Israel Patent Application No. 147049 filed Dec. 12, 2001, the contents of which are hereby incorporated by reference.

### FIELD AND BACKGROUND OF THE INVENTION

The present invention relates to a solid-fluid composition and, more particularly, to a nanostructure and liquid composition having the nanostructure and characterized by a plurality of distinguishing physical, chemical and biological characteristics.

Nanoscience is the science of small particles of materials and is one of the most important research frontiers in modern science. These small particles are of interest from a fundamental view point since all properties of a material, such as its melting point and its electronic and optical properties, change when the of the particles that make up the material become nanoscopic. With new properties come new opportunities for technological and commercial development, and applications of nanoparticles have been shown or proposed in areas as diverse as micro- and nanoelectronics, nanofluidics, coatings and paints and biotechnology.

For example, much industrial and academic effort is presently directed at the development of integrated micro devices or systems combining electrical, mechanical and/or optical/electrooptical components, commonly known as Micro Electro Mechanical Systems (MEMS). MEMS are fabricated using integrated circuit batch processing techniques and can range in size from micrometers to millimeters. These systems can sense, control and actuate on the micro scale, and function individually or in arrays to generate effects on the macro scale.

In the area of biotechnology, nanoparticles are frequently used in nanometer-scale tools for probing the real-space structures and functions of biological molecules. Auxiliary nanoparticles, such as calcium alginate nanospheres, are also used as helpers to improve gene transfection.

In metal nanoparticles, resonant collective oscillations of conduction electrons, also known as particle plasmons, are excited by an optical field. The resonance frequency of a particle plasmon is determined mainly by the dielectric function of the metal, the surrounding medium and by the shape of the particle. Resonance leads to a narrow spectrally selective absorption and an enhancement of the local field confined on and close to the surface of the metal particle. When the laser wavelength is tuned to the plasmon resonance frequency of the particle, the local electric field in proximity to the nanoparticles could be enhanced by several orders of magnitude.

Hence, nanoparticles are used for absorbing or refocusing electromagnetic radiation in proximity to a cell or a molecule, e.g., for the purpose of identification of individual molecules in biological tissue samples, in a similar fashion to the traditional fluoresce labeling.

The special radiation absorption characteristics of nanoparticles are also exploited in the area of solar energy conversion, where gallium selenide nanoparticles are used for selectively absorbing electromagnetic radiation in the visible range while reflecting electromagnetic radiation at the red end of the spectrum, thereby significantly increasing the conversion efficiency.

An additional area in which nanoscience can play a role is related to heat transfer. Despite considerable previous research and development focusing on industrial heat transfer requirements, major improvements in cooling capabilities have been held back because of a fundamental limit in the heat transfer properties of conventional fluids. It is well known that materials in solid form have orders-of-magnitude larger thermal conductivities than those of fluids. Therefore, fluids containing suspended solid particles are expected to display significantly enhanced thermal conductivities relative to conventional heat transfer fluids.

Low thermal conductivity is a primary limitation in the development of energy-efficient heat transfer fluids required in many industrial applications. To overcome this limitation, a new class of heat transfer fluids called nanofluids has been developed. These nanofluids are typically liquid compositions in which a considerable amount of nanoparticles is by suspended in liquids such as water, oil or ethylene glycol. The resulting nanofluids possess extremely high thermal conductivities compared to the liquids without dispersed nanoparticles.

Numerous theoretical and experimental studies of the effective thermal conductivity of dispersions containing particles have been conducted since Maxwell's theoretical work was published more than 100 years ago. However, all previous studies of the thermal conductivity of suspensions have been confined to those containing millimeter- or micron-sized particles. Maxwell's model shows that the effective thermal conductivity of suspensions containing spherical particles increases with the volume fraction of the solid particles. It is also known that the thermal conductivity of suspensions increases with the ratio of the surface area to volume of the particle. Since the surface area to volume ratio is 1000 times larger for particles with a 10 nm diameter than for particles with a 10 mm diameter, a much more dramatic improvement in effective thermal conductivity is expected as a result of decreasing the particle size in a solution than can obtained by altering the particle shapes of large particles.

Traditionally, nanoparticles are synthesized from a molecular level up, by the application of arc discharge, laser evaporation, pyrolysis process, use of plasma, use of sol gel and the like. Widely used nanoparticles are the fullerene carbon nanotubes, which broadly defined as objects having a diameter below about 1 μm. In a narrower sense of the words, a material having the carbon hexagonal mesh sheet of carbon substantially in parallel with the axis is called a carbon nanotube, and one with amorphous carbon surrounding a carbon nanotube is also included within the category of carbon nanotube.

Also known in the art are nanoshells which are nanoparticles having a dielectric core and a conducting shall layer. Similarly to the carbon nanotubes, the nanoshells are also manufactured from a molecular level up, for example, by bonding atoms of metal on a dielectric substrate. Nanoshells are particularly useful in applications in which it is desired to exploit the above mention optical field enhancement phenomenon. Nanoshells, however, are known to be useful only in cases of near infrared wavelengths applications.

It is recognized that nanoparticles produced from a molecular level up tends to loose the physical properties of characterizing the bulk, unless further treatment is involved in the production process. As can be understood from the above non-exhaustive list of potential applications in which nanoparticles are already in demand, there is a large diversity of physical properties which are to be considered when producing nanoparticles. In particular, nanoparticles retaining physical properties of larger, micro-sized, particles are of utmost importance.

Among the diversity of fields in which the present invention finds uses is the field of molecular biology based research and diagnostics.

Over the past ten years, as biological and genomic research have revolutionized the understanding of the molecular basis of life, it has become increasingly clear that the temporal and spatial expression of genes is responsible for all life's processes. Science has progressed from an understanding of how single genetic defects cause the traditionally recognized hereditary disorders to a realization of the importance of the interaction of multiple genetic defects along with environmental factors of more complex disorders.

This understanding has become possible with the aid of nucleic acid amplification techniques. In particular, polymerase chain reaction (PCR) has found extensive application in various fields including the diagnosis of genetic disorders, the detection of nucleic acid sequences of pathogenic organisms in clinical samples, the genetic identification of forensic samples, the analysis of mutations in activated oncogenes and other genes, and the like. In addition, PCR amplification is being used to carry out a variety of tasks in molecular cloning and analysis of DNA. These tasks include the generation of specific sequences of DNA for cloning or use as probes, the detection of segments of DNA for genetic mapping, the detection and analysis of expressed sequences by amplification of particular segments of cDNA, the generation of libraries of cDNA from small amounts of mRNA, the generation of large amounts of DNA for sequencing, the analysis of mutations, and for chromosome crawling. It is expected that PCR, as well as other nucleic acid amplification techniques, will find increasing application in many other aspects of molecular biology.

As is well-known, a strand of DNA is comprised of four different nucleotides, as determined by their bases: Adenine, Thymine, Cytosine and Guanine, respectively designated A, T, C, G. For each strand of DNA, there is a homologous strand in which A pairs with T, and C pairs with G. A specific sequence of bases which codes for a protein is referred to as a gene and genes are often segmented into regions which are responsible for protein compositions (exons) and regions which do not directly contribute to protein composition (introns).

The PCR, described generally in U.S. Pat. No. 4,683,195, allows in vitro amplification of target DNA fragments laying between two regions of known sequence. Double stranded target DNA is first melted to separate the DNA strands, and then oligonucleotide are annealed to the template DNA. The primers are chosen in such a way that they are complementary and hence specifically bind to desired, preselected positions at the 5′ and 3′ boundaries of the desired target fragments.

The oligonucleotides serve as primers for the synthesis of new complementary DNA strands using a DNA polymerase enzyme and a process known as primer extension. The orientation of the primers with respect to one another is such that the 5′ to 3′ extension product from each primer contains, when extended far enough, the sequence which is complementary to the other oligonucleotide. Thus, each newly synthesized DNA strand becomes a template for synthesis of another DNA strand beginning with the other oligonucleotide as primer. The cycle of (i) melting, (ii) annealing of oligonucleotide primers, and (iii) primer extension, can be repeated a great number of times resulting in an exponential amplification of the target fragment in between the primers.

In prior art PCR techniques, the reaction must be carried out in a reaction buffer containing a DNA polymerase cofactor. A DNA polymerase cofactor is a non-protein compound on which the enzyme depends for activity. Without the presence of the cofactor the enzyme is catalytically inactive. Known cofactors include compounds containing manganese or magnesium in such a form that divalent cations are released into an aqueous solution. Typically these cofactors are in a form of manganese or magnesium salts, such as chlorides, sulfates, acetates and fatty acid salts.

The use of a buffer with a low concentration of cofactors results in mispriming and amplification of non-target sequences. Conversely, too high of a concentration reduces primer annealing and results in inefficient DNA amplification. In addition, thermostable DNA polymerases, such as Thermus aquaticus (Taq) DNA polymerase, are magnesium-dependent. Therefore, a precise concentration of magnesium ions is necessary to maximize the efficiency of the polymerase and the specificity of the reaction.

Over the years, many attempts have been made to optimize the PCR, inter alia, by a proper selection of the primer length and sequence, annealing temperatures, length of amplificate, concentration of buffers and reaction supplements and the like. As the number of variants which are responsible to the efficiency of the PCR is extremely large, it is extremely difficult to find an optimal set of parameters for all the components participating in the process.

As further detailed in the following sections, the efficiency of nucleic acid amplification techniques can be significantly improved with the aid of a liquid composition incorporating nanostructures therein.

### SUMMARY OF THE INVENTION

According to one aspect of the present invention there is provided a nanostructure comprising a core material of a nanometric size surrounded by an envelope of ordered fluid molecules, the core material and the envelope of ordered fluid molecules being in a steady physical state.

According to another aspect of the present invention there is provided a liquid composition comprising a liquid and nanostructures, each of the nanostructures comprises a core material of a nanometric size surrounded by an envelope of ordered fluid molecules, the core material and the envelope of ordered fluid molecules being in a steady physical state; the nanostructures are designed such that when the liquid composition is first contacted with a surface and then washed by a predetermined wash protocol, an electrochemical signature of the composition is preserved on the surface.

According to yet another aspect of the present invention there is provided a liquid composition comprising a liquid and nanostructures, the liquid composition facilitates increment of bacterial colony expansion rate, whereby each of the nanostructures comprises a core material of a nanometric size surrounded by an envelope of ordered fluid molecules, the core material and the envelope of ordered fluid molecules being in a steady physical state.

According to still another aspect of the present invention there is provided a liquid composition comprising a liquid and nanostructures, the liquid composition facilitates increment of phage-bacteria or virus-cell interaction, whereby each of the nanostructures comprises a core material of a nanometric size surrounded by an envelope of ordered fluid molecules, the core material and the envelope of ordered fluid molecules being in a steady physical state.

According to an additional aspect of the present invention there is provided a liquid composition comprising a liquid and nanostructures, the liquid composition is characterized by a zeta potential which is substantial larger than a zeta potential of the liquid per se, whereby each of the nanostructures comprises a core material of a nanometric size surrounded by an envelope of ordered fluid molecules, the core material and the envelope of ordered fluid molecules being in a steady physical state.

According to yet an additional aspect of the present invention there is provided a liquid composition comprising a liquid and nanostructures, each of the nanostructures comprises a core material of a nanometric size surrounded by an envelope of ordered fluid molecules, the core material and the envelope of ordered fluid molecules being in a steady physical state, and each of the nanostructures having a specific gravity lower than or equal to a specific gravity of the liquid.

According to still further features in the described preferred embodiments the nanostructures are designed such that when the liquid composition is mixed with a dyed solution, spectral properties of the dyed solution is substantially changed.

According to still an additional aspect of the present invention there is provided a liquid composition comprising a liquid and nanostructures, each of the nanostructures comprises a core material of a nanometric size surrounded by an envelope of ordered fluid molecules, the core material and the envelope of ordered fluid molecules being in a steady physical state; the nanostructures are designed such that when the liquid composition is mixed with a dyed solution, spectral properties of the dyed solution is substantially changed.

According to yet a further aspect of the present invention there is provided a liquid composition comprising a liquid and nanostructures, the liquid composition enhances macromolecule binding to solid phase matrix, whereby each of the nanostructures comprises a core material of a nanometric size surrounded by an envelope of ordered fluid molecules, the core material and the envelope of ordered fluid molecules being in a steady physical state.

According to further features in preferred embodiments of the invention described below, the composition wherein the solid phase matrix is hydrophilic.

According to still further features in the described preferred embodiments the solid phase matrix is hydrophobic.

According to still further features in the described preferred embodiments the solid phase matrix comprises hydrophobic regions and hydrophilic regions.

According to still further features in the described preferred embodiments the macromolecule is an antibody.

According to still further features in the described preferred embodiments the antibody is a polyclonal antibody.

According to still further features in the described preferred embodiments the macromolecule comprises at least one carbohydrate hydrophilic region.

According to still further features in the described preferred embodiments the macromolecule comprises at least one carbohydrate hydrophobic region.

According to still further features in the described preferred embodiments the macromolecule is a lectin.

According to still further features in the described preferred embodiments the macromolecule is a DNA molecule.

According to still further features in the described preferred embodiments the macromolecule is an RNA molecule.

According to still a further aspect of the present invention there is provided a liquid composition comprising a liquid and nanostructures, the liquid composition is capable of at least partially de-fold DNA molecules, whereby each of the nanostructures comprises a core material of a nanometric size surrounded by an envelope of ordered fluid molecules, the core material and the envelope of ordered fluid molecules being in a steady physical state.

According to still a further aspect of the present invention there is provided a liquid composition comprising a liquid and nanostructures, the liquid composition is capable of stabilizing enzyme activity, whereby each of the nanostructures comprises a core material of a nanometric size surrounded by an envelope of ordered fluid molecules, the core material and the envelope of ordered fluid molecules being in a steady physical state.

According to further features in preferred embodiments of the invention described below, the enzyme activity is of an unbound enzyme.

According to still further features in the described preferred embodiments the enzyme activity is of a bound enzyme.

According to still further features in the described preferred embodiments the enzyme activity is of an enzyme selected from the group consisting of Alkaline Phosphatase, and β-Galactosidase.

According to still a further aspect of the present invention there is provided a liquid composition comprising a liquid and nanostructures, the liquid composition is capable of improving affinity binding of nucleic acids to a resin and improving gel electrophoresis separation, whereby each of the nanostructures comprises a core material of a nanometric size surrounded by an envelope of ordered fluid molecules, the core material and the envelope of ordered fluid molecules being in a steady physical state.

According to still a further aspect of the present invention there is provided a liquid composition comprising a liquid and nanostructures, the liquid composition is capable of increasing a capacity of a column, whereby each of the nanostructures comprises a core material of a nanometric size surrounded by an envelope of ordered fluid molecules, the core material and the envelope of ordered fluid molecules being in a steady physical state.

According to still a further aspect of the present invention there is provided a liquid composition comprising a liquid and nanostructures, the liquid composition is capable of improving efficiency of nucleic acid amplification process, whereby each of the nanostructures comprises a core material of a nanometric size surrounded by an envelope of ordered fluid molecules, the core material and the envelope of ordered fluid molecules being in a steady physical state.

According to further features in preferred embodiments of the invention described below, the nucleic acid amplification process is a polymerase chain reaction.

According to still further features in the described preferred embodiments the composition is capable of enabling catalytic activity of a DNA polymerase of said polymerase chain reaction.

According to still further features in the described preferred embodiments the polymerase chain reaction is magnesium free.

According to still further features in the described preferred embodiments the polymerase chain reaction is manganese free.

According to still a further aspect of the present invention there is provided a kit for polymerase chain reaction, comprising, in separate packaging (a) a thermostable DNA polymerase; and (b) a liquid composition having a liquid and nanostructures, each of said nanostructures comprising a core material of a nanometric size surrounded by an envelope of ordered fluid molecules, said core material and said envelope of ordered fluid molecules being in a steady physical state.

According to further features in preferred embodiments of the invention described below, the kit further comprises at least one dNTP.

According to still further features in the described preferred embodiments the kit further comprises at least one control template DNA.

According to still further features in the described preferred embodiments the kit further comprises at least one control primer.

According to still a further aspect of the present invention there is provided a method of amplifying a DNA sequence, the method comprising (a) providing a liquid composition having a liquid and nanostructures, each of the nanostructures comprising a core material of a nanometric size surrounded by an envelope of ordered fluid molecules, the core material and the envelope of ordered fluid molecules being in a steady physical state; and (b) in the presence of the liquid composition, executing a plurality of polymerase chain reaction cycles on the DNA sequence, thereby amplifying the DNA sequence.

According to further features in preferred embodiments of the invention described below, at least a portion of the fluid molecules are in a gaseous state.

According to still further features in the described preferred embodiments the nanostructures are capable of clustering with at least one additional nanostructure.

According to still further features in the described preferred embodiments the nanostructures are capable of maintaining long range interaction with at least one additional nano structure.

According to still further features in the described preferred embodiments at least a portion of the fluid molecules are identical to molecule of the liquid.

According to still further features in the described preferred embodiments a concentration of the nanostructures is lower than 10²⁰ nanostructures per litter, more preferably lower than 10¹⁵ nanostructures per litter.

According to still further features in the described preferred embodiments the nanostructures are capable of maintaining long range interaction thereamongst.

According to still further features in the described preferred embodiments the core material is selected from the group consisting of a ferroelectric core material, a ferromagnetic core material and a piezoelectric core material.

According to still further features in the described preferred embodiments the core material is a crystalline core material.

According to still further features in the described preferred embodiments the liquid is water.

According to still further features in the described preferred embodiments the nanostructures are designed such that a contact angle between the composition and a solid surface is smaller than a contact angle between the liquid and the solid surface.

According to a further aspect of the present invention there is provided a method of producing a liquid composition from a solid powder, the method comprising: (a) heating the solid powder, thereby providing a heated solid powder; (b) immersing the heated solid powder in a cold liquid; and (c) substantially contemporaneously with the step (b), irradiating the cold liquid and the heated solid powder by electromagnetic radiation, the electromagnetic radiation being characterized by a frequency selected such that nanostructures are formed from particles of the solid powder.

According to further features in preferred embodiments of the invention described below, the solid powder comprises micro-sized particles.

According to still further features in the described preferred embodiments the micro-sized particles are crystalline particles.

According to still further features in the described preferred embodiments the nanostructures are crystalline nanostructures.

According to still further features in the described preferred embodiments the solid powder is selected from the group consisting of a ferroelectric material and a ferromagnetic material.

According to still further features in the described preferred embodiments the solid powder is selected from the group consisting of BaTiO₃, WO₃ and Ba₂F₉O₁₂.

According to still further features in the described preferred embodiments the solid powder comprises a material selected from the group consisting of a mineral, a ceramic material, glass, metal and synthetic polymer.

According to still further features in the described preferred embodiments the electromagnetic radiation is in the radiofrequency range.

According to still further features in the described preferred embodiments the electromagnetic radiation is continues wave electromagnetic radiation.

According to still further features in the described preferred embodiments the electromagnetic radiation is modulated electromagnetic radiation.

The present invention successfully addresses the shortcomings of the presently known configurations by providing a nanostructure and liquid composition having the nanostructure, which is characterized by numerous distinguishing physical, chemical and biological characteristics.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of a nanostructure, according to a preferred embodiment of the present invention;

FIG. 2a is a flowchart diagram of a method of producing a liquid composition, according to a preferred embodiment of the present invention;

FIG. 2b is a flowchart diagram of a method of amplifying a DNA sequence, according to a preferred embodiment of the present invention;

FIGS. 3a-e are TEM images of the nanostructures of the present invention;

FIG. 4 shows the effect of dye on the liquid composition of the present invention;

FIGS. 5a-b show the effect of high g centrifugation on the liquid composition, where FIG. 5a shows signals recorded of a lower portion of a tube and FIG. 5b show signals recorded of an upper portion of the tube;

FIGS. 6a-c show results of pH tests, performed on the liquid composition of the present invention;

FIG. 7 shows the absorption spectrum of the liquid composition of the present invention;

FIG. 8 show results of potential measurements of the liquid composition of the present invention;

FIGS. 9a-b show bacteriophage reaction in the presence of the liquid composition of the present invention (left) and in the presence of a control medium (right);

FIG. 10 shows a comparison between bacteriolysis surface areas of a control liquid and the liquid composition of the present invention;

FIG. 11 shows phage typing concentration at 100 routine test dilution, in the presence of the liquid composition of the present invention (left) and in the presence of a control medium (right);

FIG. 12 shows optic density as a function of time, of the liquid composition of the present invention and a control medium;

FIGS. 13a-c show optic density in slime-producing Staphylococcus epidermidis in an experiment directed to investigate the effect of the liquid composition of the present invention on the adherence of coagulase-negative staphylococci to microtiter plates;

FIG. 14 is a histogram representing 15 repeated experiments of slime adherence on different micro titer plates;

FIG. 15 shows slime adherence differences in the liquid composition of the present invention and the control on the same micro titer plate;

FIGS. 16a-c show an electrochemical deposition experimental setup;

FIGS. 17a-b show electrochemical deposition of the liquid composition of the present invention (FIG. 17a) and the control (FIG. 17b);

FIG. 18 show electrochemical deposition of reverse osmosis (RO) water in a cell which was in contact with the liquid composition of the present invention for a period of 30 minutes;

FIGS. 19a-b show results of Bacillus subtilis colony growth for the liquid composition of the present invention (FIG. 19a) and a control medium (FIG. 19b);

FIGS. 20a-c show results of Bacillus subtilis colony growth, for the water with a raw powder (FIG. 20a), reverse osmosis water (FIG. 20b) and the liquid composition of the present invention (FIG. 20c);

FIGS. 21a-d show bindings of labeled and non-labeled antibodies to medium costar microtitration plate (FIG. 21a), non-sorp microtitration plate (FIG. 21b), maxisorp microtitration plate (FIG. 21c) and polysorp microtitration plate (FIG. 21d), using the liquid composition of the present invention and control buffer;

FIGS. 22a-d show bindings of labeled antibodies to medium costar microtitration plate (FIG. 22a), non-sorp microtitration plate (FIG. 22b), maxisorp microtitration plate (FIG. 22c) and polysorp microtitration plate (FIG. 22d), using the liquid composition of the present invention and control buffer;

FIGS. 23a-d show bindings of labeled antibodies after overnight incubation at 4° C., to non-sorp microtitration plate (FIG. 23a), medium costar microtitration plate (FIG. 23b), polysorp microtitration plate (FIG. 23c) and maxisorp microtitration plate (FIG. 23d), using the liquid composition of the present invention and using buffer;

FIGS. 24a-d show bindings of labeled antibodies after 2 hours incubation at 37° C., to non-sorp microtitration plate (FIG. 24a), medium costar microtitration plate (FIG. 24b), polysorp microtitration plate (FIG. 24c) and maxisorp microtitration plate (FIG. 24d), using the liquid composition of the present invention and using control buffer;

FIGS. 25a-d show bindings of labeled and non-labeled antibodies after overnight incubation at 4° C., to medium costar microtitration plate (FIG. 25a), polysorp microtitration plate (FIG. 25b), maxisorp microtitration plate (FIG. 25c) and non-sorp microtitration plate (FIG. 25d), using the liquid composition of the present invention and control buffer;

FIGS. 26a-d show bindings of labeled and non-labeled antibodies after overnight incubation at room temperature, to medium costar microtitration plate (FIG. 25a), polysorp microtitration plate (FIG. 25b), maxisorp microtitration plate (FIG. 25c) and non-sorp microtitration plate (FIG. 25d), using the liquid composition of the present invention and control buffer;

FIGS. 27a-b show binding results of labeled and non-labeled antibodies (FIG. 27a) and only labeled antibodies (FIG. 27b) using phosphate washing buffer, for the liquid composition of the present invention and control buffer;

FIGS. 27c-d show binding results of labeled and non-labeled antibodies (FIG. 27a) and only labeled antibodies (FIG. 27b) using PBS washing buffer, for the liquid composition of the present invention and control buffer;

FIGS. 28a-b show bindings of labeled and non-labeled antibodies (FIG. 28a) and only labeled antibodies (FIG. 28a), after overnight incubation at 4° C., to medium costar microtitration plate, using the liquid composition of the present invention and control buffer;

FIGS. 29a-c show bindings of labeled lectin to non-sorp microtitration plate for acetate (FIG. 29a), carbonate (FIG. 29b) and phosphate (FIG. 29c) buffers, using the liquid composition of the present invention and control buffer;

FIGS. 30a-d show bindings of labeled lectin to maxisorp microtitration plate for carbonate (FIGS. 30a-b), acetate (FIG. 30c) and phosphate (FIG. 30d) buffers, using the liquid composition of the present invention and control buffer, where the graph shown in FIG. 30b is a linear portion of the graph shown in FIG. 30a;

FIGS. 31a-b show average binding enhancement capability of the liquid composition of the present invention for nucleic acid;

FIGS. 32-35b are images of PCR product samples before and after purifications for different buffer combinations and different elution steps;

FIGS. 36-37 are an image (FIG. 36) and quantitative analysis (FIG. 37) of columns having PCR product solutions in different amounts, concentrations and elution steps;

FIGS. 38a-c are image of columns having mixtures of columns 5-17 shown in FIG. 36, for three elution steps;

FIG. 39a shows the area of control buffer (designated CO) and the liquid composition of the present invention (designated LC) as a function of the loading volume for each of the three elution steps of FIGS. 38a-c;

FIG. 39b shows the ratio LC/CO as a function of the loading volume for each of the three elution steps of FIGS. 38a-c;

FIGS. 40a-42b are lane images comparing the migration speed of DNA in gel electrophoresis experiments in the presence of RO water (FIGS. 40a, 41a and 42a) and in the presence of the liquid composition of the present invention (FIGS. 40b, 41b and 42b);

FIGS. 43a-45d are lane images captured in gel electrophoresis experiments in which the effect of the liquid composition of the present invention on running buffer was investigated;

FIGS. 46a-48d are lane images captured in gel electrophoresis experiments in which the effect of the liquid composition of the present invention on the gel buffer was investigated;

FIG. 49 show values of a stability enhancement parameter, Sₑ, as a function of the dilution, in an experiment in which the effect of the liquid composition of the present invention on the activity and stability of unbound form of alkaline phosphatase was investigated;

FIG. 50 show enzyme activity of alkaline phosphatase bound to Strept-Avidin, diluted in RO water and the liquid composition of the present invention as a function of the dilution, in an experiment in which the effect of the liquid composition of the present invention on the activity and stability of bound form of alkaline phosphatase was investigated;

FIGS. 51a-d show stability of β-Galactosidase after 24 hours (FIG. 51a), 48 hours (FIG. 51b), 72 hours (FIG. 51c) and 120 hours (FIG. 51d), in an experiment in which the effect of the liquid composition of the present invention on the activity and stability of β-Galactosidase was investigated;

FIGS. 52a-d show values of a stability enhancement parameter, Sₑ, after 24 hours (FIG. 52a), 48 hours (FIG. 52b), 72 hours (FIG. 52c) and 120 hours (FIG. 52d), in the experiment in which the effect of the liquid composition of the present invention on the activity and stability of β-Galactosidase was investigated;

FIG. 53a show remaining activity of alkaline phosphatase after drying and heat treatment; and

FIG. 53b show values of the stability enhancement parameter, Sₑ, of alkaline phosphatase after drying and heat treatment.

### DESCRIPTION OF THE PREFERRED EMBODIMENTS

The present invention is of a nanostructure and liquid composition having the nanostructure and characterized by a plurality of distinguishing physical, chemical and biological characteristics. The liquid composition of the present invention can be used for many biological and chemical application such as, but not limited to, bacterial colony growth, electrochemical deposition and the like

The principles of a nanostructure and liquid composition according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

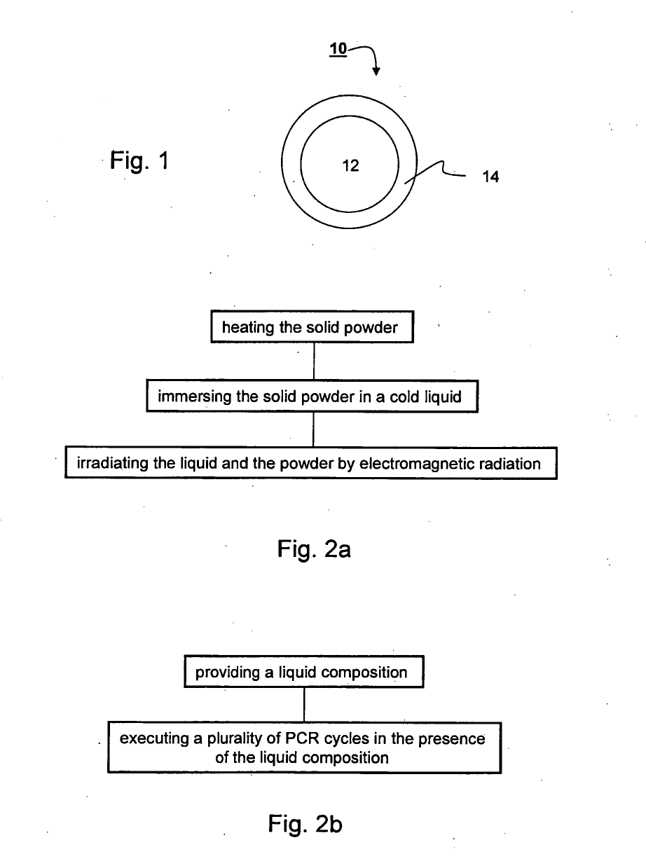

Referring now to the drawings, FIG. 1 illustrates a nanostructure 10 comprising a core material 12 of a nanometric size, surrounded by an envelope 14 of ordered fluid molecules. Core material 12 and envelope 14 are in a steady physical state.

As used herein the phrase “steady physical state” is referred to a situation in which objects or molecules are bound by any potential having at least a local minimum. Representative examples, for such a potential include, without limitation, Van der Waals potential, Yukawa potential, Lenard-Jones potential and the like. Other forms of potentials are also contemplated.

As used herein the phrase “ordered fluid molecules” is referred to an organized arrangement of fluid molecules having correlations thereamongst.

As used herein the term “about” refers to ±10%.

According to a preferred embodiment of the present invention, the fluid molecules of envelope 14 may be either in a liquid state or in a gaseous state. As further demonstrated in the Example section that follows (see Example 3), when envelope 14 comprises gaseous material, the nanostructure is capable of floating when subjected to sufficient g-forces.

Core material 12 is not limited to a certain type or family of materials, and can be selected in accordance with the application for which the nanostructure is designed. Representative examples include, without limitation, ferroelectric material, a ferromagnetic material and a piezoelectric material. As demonstrated in the Examples section that follows (see Example 1) core material 12 may also have crystalline structure.

A ferroelectric material is a material that maintains, over some temperature range, a permanent electric polarization that can be reversed or reoriented by the application of an electric field. A ferromagnetic material is a material that maintains permanent magnetization, which is reversible by applying a magnetic field. According to a preferred embodiment of the present invention, when core material 12 is ferroelectric or ferromagnetic, nanostructure 10 retains its ferroelectric or ferromagnetic properties. Hence, nanostructure 10 has a particular feature in which macro scale physical properties are brought into a nanoscale environment.

According to a preferred embodiment of the present invention nanostructure 10 is capable of clustering with at least one additional nanostructure. More specifically, when a certain concentration of nanostructure 10 is mixed in a liquid (e.g., water), attractive electrostatic forces between several nanostructures may cause adherence thereamongst so as to form a cluster of nano structures. Preferably, even when the distance between the nanostructures prevents cluster formation, nanostructure 10 is capable of maintaining long range interaction (about 0.5-10 μm), with the other nanostructures. Long range interaction between nanostructures present in liquid, induces unique characteristics on the liquid, which can be exploited in many applications, such as, but not limited to, biological and chemical assays.

The unique properties of nanostructure 10 may be accomplished, for example, by producing nanostructure 10 using a “top-down” process. More specifically, nanostructure 10 can be produced from a raw powder of micro-sized particles, say, above 1 μm or above 10 μm in diameter, which are broken in a controlled manner, to provide nanometer-sized particles. Typically, such a process is performed in a cold liquid (preferably, but not obligatory, water) into which high-temperature raw powder is inserted, under condition of electromagnetic radiofrequency (RF) radiation.

A more detailed description of the production process, is preceded by the following review of the physical properties of water, which, as stated, is the preferred liquid.

Hence, water is one of a remarkable substance, which has been very well studied. Although it appears to be a very simple molecule consisting of two hydrogen atoms attached to an oxygen atom, it has complex properties. Water has numerous special properties due to hydrogen bonding, such as high surface tension, high viscosity, and the capability of forming ordered hexagonal, pentagonal of dodecahedral water arrays by themselves of around other substances.

The melting point of water is over 100 K higher than expected when considering other molecules with similar molecular weight. In the hexagonal ice phase of the water (the normal form of ice and snow), all water molecules participate in four hydrogen bonds (two as donor and two as acceptor) and are held relatively static. In liquid water, some hydrogen bonds must be broken to allow the molecules move around. The large energy required for breaking these bonds must be supplied during the melting process and only a relatively minor amount of energy is reclaimed from the change in volume. The free energy change must be zero at the melting point. As temperature is increased, the amount of hydrogen bonding in liquid water decreases and its entropy increases. Melting will only occur when there is sufficient entropy change to provide the energy required for the bond breaking. The low entropy (high organization) of liquid water causes this melting point to be high.

Most of the water properties are attributed to the above mentioned hydrogen bonding occurring when an atom of hydrogen is attracted by rather strong forces to two oxygen atoms (as opposed to one), so that it can be considered to be acting as a bind between the two atoms.

Water has high density, which increases with the temperature, up to a local maximum occurring at a temperature of 3.984° C. This phenomenon is known as the density anomaly of water. The high density of liquid water is due mainly to the cohesive nature of the hydrogen-bonded network. This reduces the free volume and ensures a relatively high-density, compensating for the partial open nature of the hydrogen-bonded network. The anomalous temperature-density behavior of water can be explained utilizing the range of environments within whole or partially formed clusters with differing degrees of dodecahedral puckering.

The density maximum (and molar volume minimum) is brought about by the opposing effects of increasing temperature, causing both structural collapse that increases density and thermal expansion that lowers density. At lower temperatures, there is a higher concentration of expanded structures whereas at higher temperatures there is a higher concentration of collapsed structures and fragments, but the volume they occupy expands with temperature. The change from expanded structures to collapsed structures as the temperature rises is accompanied by positive changes in entropy and enthalpy due to the less ordered structure and greater hydrogen bond bending, respectively.

Generally, the hydrogen bonds of water create extensive networks, that can form numerous hexagonal, pentagonal of dodecahedral water arrays. The hydrogen-bonded network possesses a large extent of order. Additionally, there is temperature dependent competition between the ordering effects of hydrogen bonding and the disordering kinetic effects.

As known, water molecules can form ordered structures and superstructures. For example, shells of ordered water form around various biomolecules such as proteins and carbohydrates. The ordered water environment around these biomolecules are strongly involved in biological function with regards to intracellular function including, for example, signal transduction from receptors to cell nucleus. Additionally these water structures are stable and can protect the surface of the molecule.

Most of the ordered structure of liquefied water is on a short-range scale, typically about 1 nm. Although long-range order may, in principle exists, when the water is in its liquid phase, such long-range order has extremely low probability to occur spontaneously, because molecules in a liquid state are in constant thermal motion. Due to the hydrogen bonding and the non-bonding interactions, water molecules can form an infinite hydrogen-bonded network with specific and structured clustering. Thus, small clusters of water molecules can form water octamers that can further cluster with other smaller clusters to form icosahedral water clusters consisting of hundreds of water molecules. Therefore, water molecules can form ordered structures.

Other water properties include high boiling point, high critical point, reduction of the melting point with pressure (the pressure anomaly), compressibility which decreases with increasing temperature up to a minimum at about 46° C., and the like.

The unique properties of water have been exploited by the Inventor of the present invention for the purpose of producing nanostructure 10. Thus, according to another aspect of the present invention there is provided a method of producing a liquid composition.

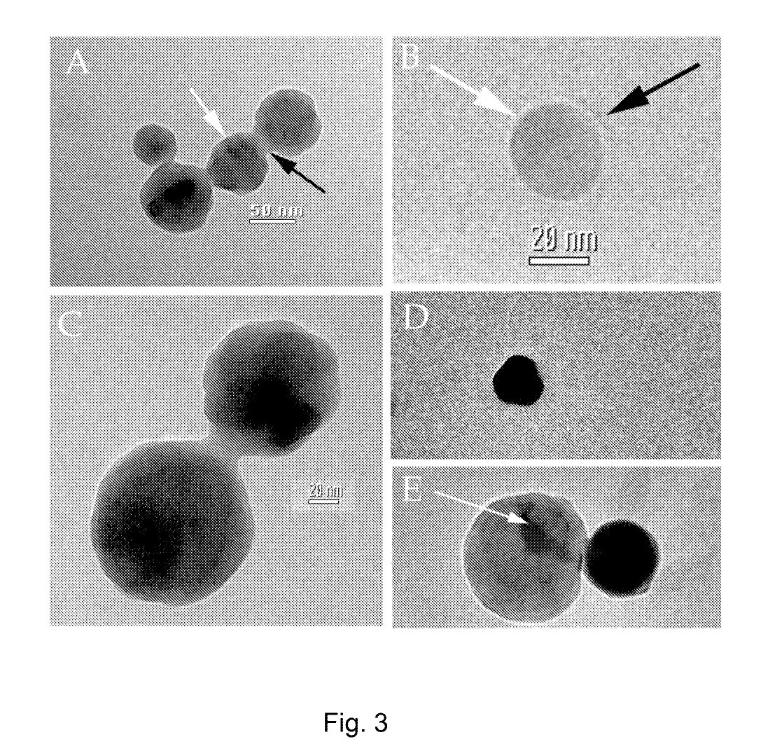

Reference is now made to FIG. 2a which is a flowchart diagram of the method, according to a preferred embodiment of the present invention. The method comprises the following method steps, in which in a first step, a solid powder (e.g., a mineral, a ceramic powder, a glass powder, a metal powder, a synthetic polymer, etc.) is heated, to a sufficiently high temperature, preferably more than about 700° C. Representative examples of solid powder which are contemplated include, without limitation, BaTiO₃, WO₃ and Ba₂F₉O₁₂. In a second step, the heated powder is immersed in a cold liquid, preferably water below its density anomaly temperature, e.g., 3° C. or 2° C. In a third step of the method, which is preferably executed substantially contemporaneously with the second step, the cold liquid and the powder are irradiated by electromagnetic RF radiation, preferably above 500 MHz, which may be either continues wave RF radiation or modulated RF radiation.

The formation of the nanostructures in the liquid may be explained as follows. The combination of cold liquid, and RF radiation (i.e., highly oscillating electromagnetic field) influences the interface between the particles and the liquid, breaks the liquid molecules and the particles. The broken liquid molecules are in the form of free radicals, which envelope the (nano-sized) debris of the particles. Being at a small temperature, the free radicals and the debris enter a steady physical state. The attraction of the free radicals to the nanostructures can be understood from the relatively small size of the nanostructures, compared to the correlation length of the liquid molecules. It has been argued [D. Bartolo, et al., Europhys. Lett., 2000, 49(6):729-734], that a small size perturbation may contribute to a pure Casimir effect, which is manifested by long-range interactions.

Performing the above method according to present invention successfully produces the nanostructure of the present invention. In particular, the above method allows the formation of envelope 14 as further detailed hereinabove. Thus, according to another aspect of the present invention, there is provided a liquid composition having a liquid and nanostructures 10. When the liquid composition is manufactured by the above method, with no additional steps, envelope 14 of nanostructure 10 is preferably made of molecules which are identical to the molecule of the liquid. Alternatively, the nanostructure may be further mixed (with or without RF irradiation) with a different liquid, so that in the final composition, at least a portion of envelope 14 is made of molecules which are different than the molecules of the liquid. Due to the formation of envelope 14 the nanostructures preferably has a specific gravity which is lower than or equal to a specific gravity of liquid.

The concentration of the nanostructures is not limited. A preferred concentration is below 10²⁰ nanostructures per litter, more preferably below 10¹⁵ nanostructures per litter. One ordinarily skilled in the art would appreciate that with such concentrations, the average distance between the nanostructures in the composition is rather large, of the order of microns. As further detailed hereinunder and demonstrated in the Example section that follows, the liquid composition of the present invention has many unique characteristics. These characteristics may be facilitated, for example, by long range interactions between the nanostructures. In particular, long range interactions allow that employment of the above relatively low concentrations.

Interactions between the nanostructures (both long range and short range interaction) facilitate self organization capability of the liquid composition, similar to a self organization of bacterial colonies. When a bacterial colony grows, self-organization allows it to cope with adverse external conditions and to “collectively learn” from the environment for improving the growth rate. Similarly, the long range interaction and thereby the long range order of the liquid composition allows the liquid composition to perform self-organization, so as to adjust to different environmental conditions, such as, but not limited to, different temperatures, electrical currents, radiation and the like.

The long range order of the liquid composition of the present invention is best seen when the liquid composition is subjected to an electrochemical deposition (ECD) experiment (see also Example 9 in the Examples section that follows).

ECD is a process in which a substance is subjected to a potential difference (for example using two electrodes), so that electrochemical process is initiated. A particular property of the ECD process is the material distribution obtained thereby. During the electrochemical process, the potential measured between the electrodes at a given current, is the sum of several types of over-voltage and the Ohmic drop in the substrate. The size of the Ohmic drop depends on the conductivity of the substrate and the distance between the electrodes. The current density of a specific local area of an electrode is a function of the distance to the opposite electrode. This effect is called the primary current distribution, and it depends on the geometry of the electrodes and the conductivity of the substrate.

When the potential difference between the electrodes is large, compared to the equilibrium voltage, the substrates experience a transition to a non-equilibrium state, and as a result, structures of different morphologies are formed. It has been found [E. Ben-Jacob, “From snowflake formation to growth of bacterial colonies,” Cont. Phys., 1993, 34(5)] that systems in non-equilibrium states may select a morphology and/or experience transitions between two morphologies: dense branching morphology and a dendritic morphology.

According to a preferred embodiment of the present invention when the liquid composition of the present invention is placed in an electrochemical deposition cell, a predetermined morphology (e.g., dense branching and/or dendritic) is formed. Preferably, the liquid composition of the present invention is capable of preserving an electrochemical signature on the surface of the cell even when replaced by a different liquid (e.g., water). More specifically, according to a preferred embodiment of the present invention, when the liquid composition is first contacted with the surface of the electrochemical deposition cell and then washed by a predetermined wash protocol, an electrochemical signature of the composition is preserved on the surface of the cell.

An additional characteristic of the present invention is a small contact angle between the liquid composition and solid surface. Preferably, the contact angle between the liquid composition and the surface is smaller than a contact angle between liquid (without the nanostructures) and the surface. One ordinarily skilled in the art would appreciate that small contact angle allows the liquid composition to “wet” the surface in larger extent. It is to be understood that this feature of the present invention is not limited to large concentration of the nanostructures in the liquid, but rather also to low concentrations, with the aid of the above-mentioned long range interactions between the nanostructures.

While reducing the present invention to practice, it has been unexpectedly realized (see Examples 6, 7 and 10 in the Examples section that follows) that the liquid composition of the present invention is capable of facilitating increment of bacterial colony expansion rate and phage-bacteria or virus-cell interaction, even when the solid powder used for preparing the liquid composition is toxic to the bacteria. The unique process by which the liquid composition is produced, which, as stated, allows the formation of envelope 14 surrounding core material 12, significantly suppresses any toxic influence of the liquid composition on the bacteria or phages.

An additional characteristic of the liquid composition of the present invention is related to the so called zeta (ζ) potential. ζ potential is related to a physical phenomena called electrophoresis and dielectrophoresis in which particles can move in a liquid under the influence of electric fields present therein. The ζ potential is the electric potential at a shear plane, defined on the boundary between two regions of the liquid having different behaviors. The electrophoretic mobility of particles (the ratio of the velocity of particles to the field strength) is proportional to the potential.

Being a surface related quantity, the ζ potential is particularly important in systems with small particle size, where the total surface area of the particles is large relative to their total volume, so that surface related phenomena determine their behavior.

According to a preferred embodiment of the present invention, the liquid composition is characterized by a ζ potential which is substantial larger than the ζ potential of the liquid per se. Large ζ potential corresponds to enhanced mobility of the nanostructures in the liquid, hence, it may contribute, for example, to the formation of special morphologies in the electrochemical deposition process.

There are many methods of measuring the ζ potential of the liquid composition, including, without limitation, microelectrophoresis, light scattering, light diffraction, acoustics, electroacoustics etc. For example, one method of measuring ζ potential is disclosed in U.S. Pat. No. 6,449,563, the contents of which are hereby incorporated by reference.

As stated in the Background section hereinabove, the present invention also relates to the field of molecular biology research and diagnosis, particularly to nucleic acid amplification techniques, such as, but not limited to, polymerase chain reaction (PCR), ligase chain reaction (LCR), strand displacement amplification (SDA) and self-sustained sequence replication (SSSR).

It has been found by the inventor of the present invention, that the liquid composition of the present invention is capable of improving efficiency of a nucleic acid amplification process, e.g., by enabling catalytic activity of a DNA polymerase in PCR procedures. The enablement of catalytic activity is preferably achieved without the use of additional cofactors such as, but not limited to, magnesium or manganese. As will be appreciated by one of ordinary skill in the art, the ability to employ a magnesium-free or manganese-free PCR is highly advantageous. This is because the efficiency of a PCR procedure is known to be very sensitive to the concentration of the cofactors present in the reaction. An expert scientist is often required to calculate in advance the concentration of cofactors or to perform many tests, each with a different concentration of cofactors, before achieving the desired amplification efficiency.

The use of the liquid composition of the present invention thus allows the user to execute a simple and highly efficient multi-cycle PCR procedure without having to calculate or vary the concentration of cofactors in the PCR mix.

Additionally, it has been found by the present inventor that polymerase chain reaction can take place devoid of any additional buffer or liquid. One of the major problems associated with the application of PCR to clinical diagnostics is the susceptibility of PCR to carryover contamination. These are false positives due to contamination of thw sample with molecules amplified in a previous PCR. The use of the liquid composition of the present invention as a sole PCR mix significantly reduces the probability for carryover contamination, because the entire procedure can be carried out without the need to add any additional buffer or liquid, hence avoiding the risk of contamination.

Thus, according to a preferred embodiment of the present invention there is provided a kit for polymerase chain reaction. The PCR kit of the present invention may, if desired, be presented in a pack which may contain one or more units of the kit of the present invention. The pack may be accompanied by instructions for using the kit. The pack may also be accommodated by a notice associated with the container in a form prescribed by a governmental agency regulating the manufacture, use or sale of laboratory supplements, which notice is reflective of approval by the agency of the form of the compositions.

The kit comprises, preferably in separate packaging, a thermostable DNA polymerase, such as, but not limited to, Taq polymerase and the liquid composition of the present invention. Additionally, the kit may comprise at least one dNTP, such as, but not limited to, dATP, dCTP, dGTP, dTTP. Analogues such as dITP and 7-deaza-dGTP are also contemplated.

According to a preferred embodiment of the present invention the kit may further comprises at least one control template DNA and/or at least one at least one control primer to allow the user to perform at least one control test to ensure the PCR performance.

According to an additional aspect of the present invention there is provided a method of amplifying a DNA sequence, the method comprises the following method steps illustrated in the flowchart of FIG. 2b. In a first step of the method, the liquid composition of the present invention is provided, and in a second step, a plurality of PCR cycles is executed on the DNA sequence in the presence of the liquid composition.

The PCR cycles can be performed in any way known in the art, such as, but not limited to, the PCR cycles disclosed in U.S. Pat. Nos. 4,683,195, 4,683,202, 4,800,159, 4,965,188, 5,512,462, 6,007,231, 6,150,094, 6,214,557, 6,231,812, 6,391,559, 6,740,510 and International Patent application No. WO/9911823.

Preferably, in each PCR cycle, the DNA sequence is first treated to form single-stranded complementary strands. Subsequently, a pair of oligonucleotide primers which are specific to the DNA sequence are added to the liquid composition. The pair of primers is then annealed to complementary sequences on the single-stranded complementary strands. Under proper conditions, the annealed primers extend to synthesize extension products which are respectively complementary to each of the single-strands.

Beside nucleic acid amplification, the liquid composition of the present invention can be used as a buffer or an add-on to an existing buffer, for improving many chemical and biological assays and reactions.

Hence, in one embodiment the liquid composition of the present invention can be used to at least partially de-fold DNA molecules.

In another embodiment, the liquid composition of the present invention can be used to facilitate isolation and purification of DNA.

In an additional embodiment, the liquid composition of the present invention can be used for stabilizing enzyme activity of many enzymes, either bound or unbound enzymes, such as, but not limited to, Alkaline Phosphatase or β-Galactosidase.

In still another embodiment, the liquid composition of the present invention can also be used for enhancing binding of macromolecule to solid phase matrix. As further demonstrated in the Examples section that follows (see Example 11), the liquid composition of the present invention can enhance binding to both hydrophilic and hydrophobic substances. In addition, the liquid composition of the present invention can enhance binding to substances having hydrophobic regions and hydrophilic regions. The binding of many macromolecules can be enhanced to the above substances, including, without limitation macromolecule having one or more carbohydrate hydrophilic or carbohydrate hydrophobic regions, antibodies, polyclonal antibodies, lectin, DNA molecules, RNA moleculs and the like.

Additionally, as demonstrated in the Examples section that follows (see Examples 12-14), it has been found by the present inventor that the liquid composition of the present invention can be used for increasing a capacity of a column, binding of nucleic acids to a resin and improving gel electrophoresis separation.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Examples

Reference is now made to the following examples, which, together with the above descriptions, illustrate the invention in a non limiting fashion.

The Examples below are directed at various characterization experiments, which has been performed using the nanostructure and the liquid composition of the present invention. The nanostructure and the liquid composition used in the following experiments were manufactured in accordance with the present invention as further detailed hereinabove. More specifically, in the production method which was employed to provide the nanostructure and the liquid composition, the following protocol was used:

First, a powder of micro-sized BaTiO₃ was heated, to a temperature of 880° C. Second, under condition of continues wave RF radiation at a frequency of 915 MHz, the heated powder was immersed in water at a temperature of 2° C. The radiation and sudden cooling caused the micro-sized particles of the powder to break into nanostructures. Subsequently, the liquid composition (nanostructure and water) was allowed to heat to room temperature.

In the following examples, different liquid compositions, manufactured according to a preferred embodiment of the present invention, are referred to as LC1, LC2, LC3, LC4, LC5, LC6, LC7, LC8 and LC9.

### Example 1

### Solid-Fluid Coupling and Clustering of the Nanostructure

In this Example, the coupling of the surrounding fluid molecules to the core material was investigated by Cryogenic-temperature transmission electron microscopy (cryo-TEM), which is a modern technique of structural fluid systems. The analysis involved the following steps in which in a first step, the liquid composition of the present invention (LC1) was cooled ultra-rapidly, so that vitreous sample was provided, and in a second step the vitreous sample was examined in via TEM at cryogenic temperatures.

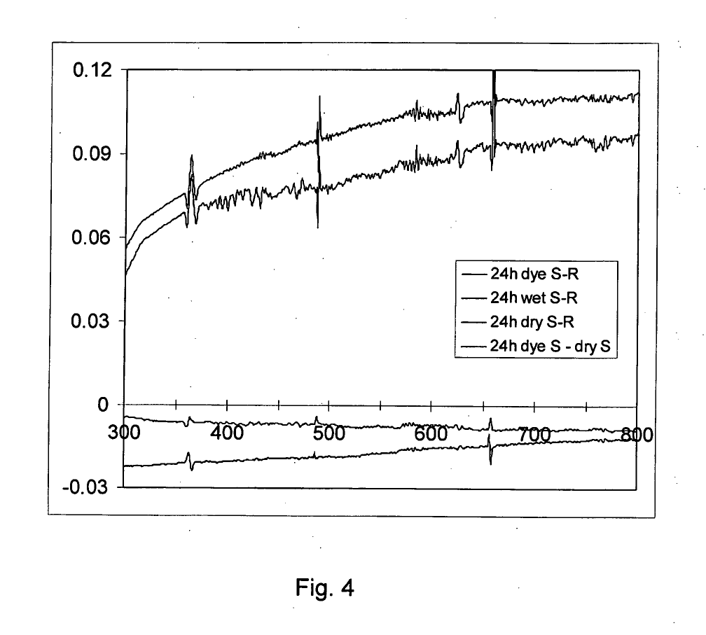

FIGS. 3a-e show TEM images of the nanostructures of the present invention. FIG. 3a is an image of a region, about 200 nm long and about 150 nm wide, occupied by a four nanostructures. As shown in FIG. 3a, the nanostructures form a cluster via intermediate regions of fluid molecules; one such region is marked by a black arrow. Striations surrounding the nanostructures, marked by a white arrow in FIG. 3a, suggest crystalline structure thereof.

FIG. 3b is an image of a single nanostructure, about 20 nm in diameter. A bright corona, marked by a white arrow, may be a consequence of an optical interference effect, commonly known as the Fresnel effect. An additional, darker, corona (marked by a black arrow in FIG. 3b) was observed at a further distance from the center of the nanostructure, as compared to the bright corona. The dark corona indelicate an ordered structure of fluid molecules surrounding the core, so that the entire nanostructure is in steady physical state.

FIGS. 3c-e are of equal magnification, which is illustrated by a scale-bar shown in FIG. 3c. FIG. 3c further demonstrates, in a larger magnification than FIG. 3a, the ability of the nanostructures of the present invention to cluster. FIG. 3d shows a single nanostructure characterized by crystalline facets and FIG. 3e shows a cluster of two nanostructures in which one is characterized by crystalline facets and the other has a well defined dark area which is also attributed to its crystalline structure.

### Example 2

### Effect of Dye on the Liquid Composition

The interaction of the liquid composition of the present invention with dye was investigated. A liquid composition, manufactured as further detailed above, was dyed with a Ru based dye (N3) dissolved in ethanol.

One cuvette containing the liquid composition of the present invention (LC1) was exposed to the dye solution for 24 hours. A second cuvette containing the liquid composition was exposed to the following protocol: (i) stirring, (ii) drying with air stream, and (iii) dying. Two additional cuvettes, containing pure water were subjected to the above tests as control groups.

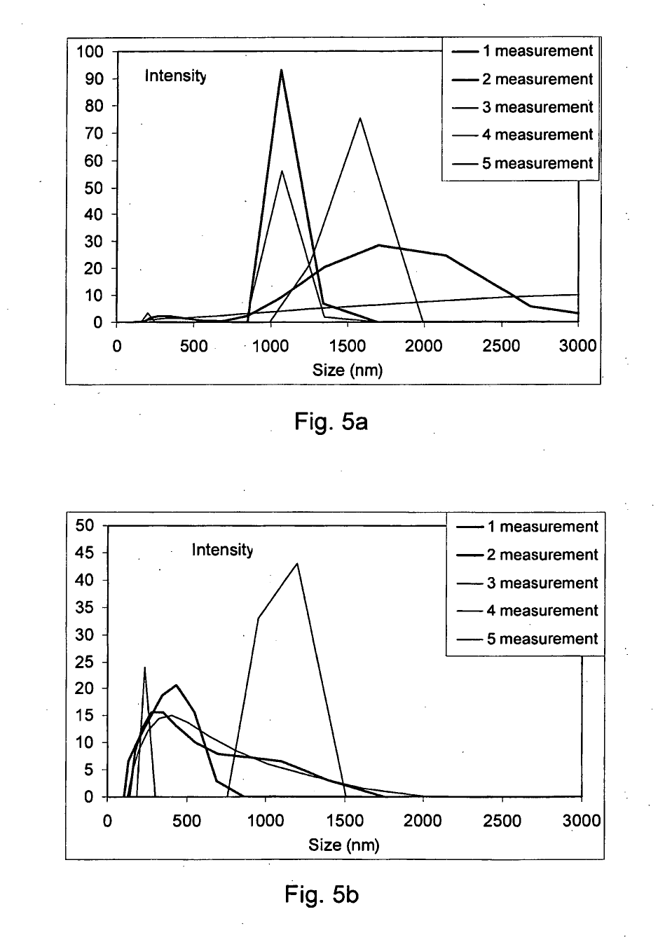

FIG. 4 shows the results of the four tests. As shown in FIG. 4 the addition of the dye result in disappearance of the dye color (see the lower curves in FIG. 4), in contrast to the case of pure water (see the lower curves in FIG. 4) where the color was maintained. Hence, the interaction with the nanostructures affects the dye spectrum by either a change of the electronic structure or dye oxidation.

The color disappearance is best evident in the picture of the cuvette. All samples presented in FIG. 4 were stirred containing the liquid composition of the present invention. The sample designated “dry S—R” was kept dry for 24 hours; the sample designated wet “S—R” was kept wet with ethanol; the sample designated “dye S—R” was dyed (dye in ethanol) and the sample designated “dye S-dry R” was dried and remeasured.

### Example 3

### Effect of High g Centrifugation on the Liquid Composition

Tubes containing the liquid composition of the present invention were centrifuged at high g values.

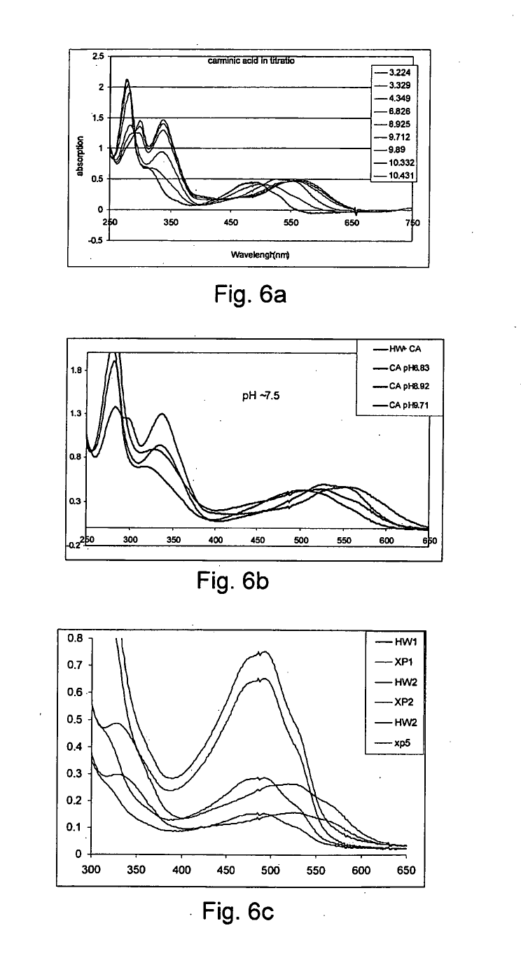

FIGS. 5a-b show results of five integrated light scattering (ILS) measurements of the liquid composition of the present invention (LC1) after centrifugation. FIG. 5a show signals recorded of the lower portion of the tubes. As shown, no signal of structures smaller that 1 μm was recorded from the lower portion. FIG. 5b shows signals recorded of the upper portion of the tubes. A clear presence of structures smaller than 1 μm is shown. In all the measurements, the location of the peaks are consistent with nanostructures of about 200-300 nm.

This experiment demonstrated that the nanostructures have a specific gravity which is lower than the specific gravity of the host liquid (water).

### Example 4

### pH Tests

The liquid composition of the present invention was subjected to two pH tests. In a first test, caraminic indicator was added to the liquid composition of the present invention (LC1) so as to provide indication of affective pH.

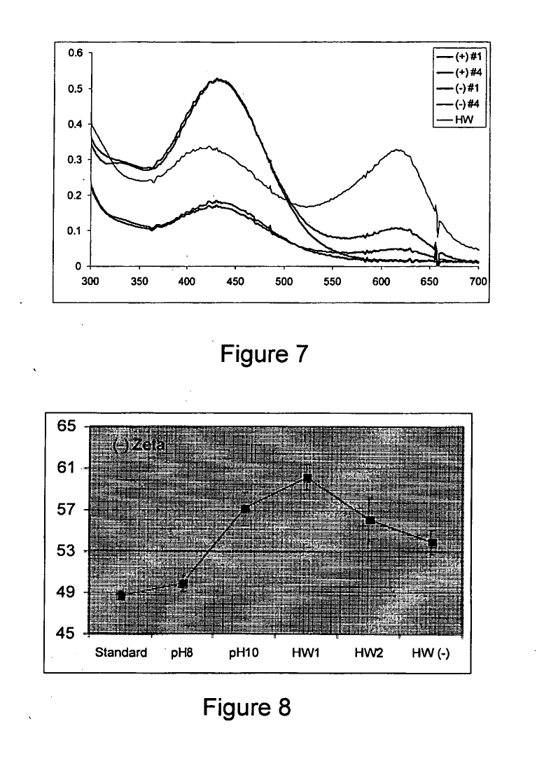

FIG. 6a shows the spectral change of the caraminic indicator during titration. These spectra are used to examine the pH of the liquid composition. FIG. 6b shows that the liquid composition spectrum is close to the spectrum of water at pH 7.5. FIG. 6c shows that unlike the original water used in the process several liquid composition samples have pH 7.5 spectra.

The results of the first test indicate that the liquid composition has a pH of 7.5, which is more than the pH value of pure water.

In a second test, Bromo Thymol Blue (BTB) was added to the liquid composition of the present invention (LC1). This indicator does not affect the pH and it changes colors in the pH range of interest.

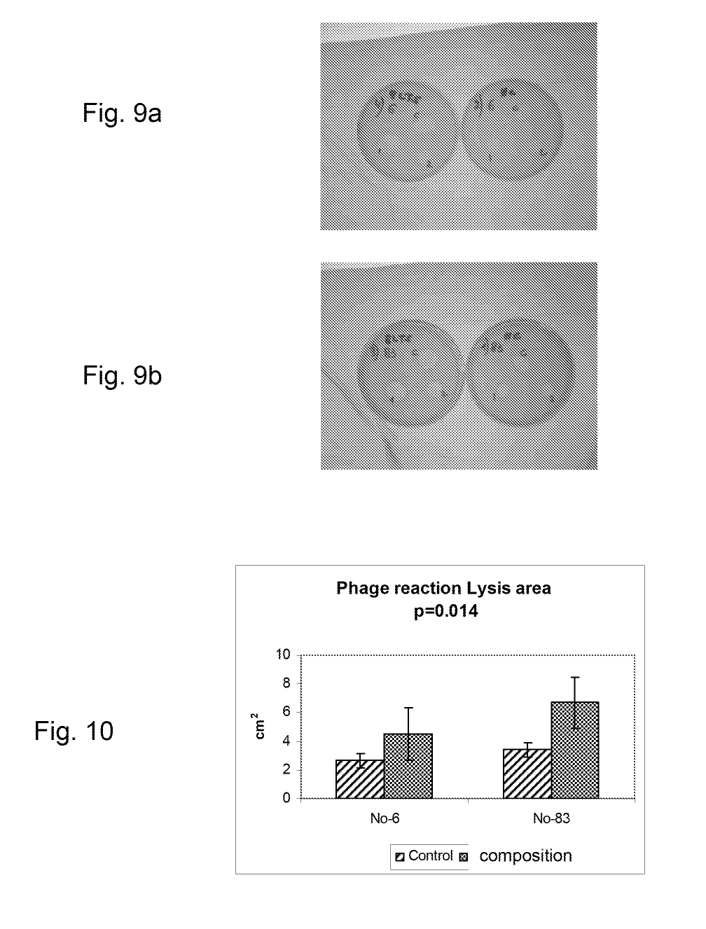

The absorption spectrum for samples No. 1 and 4 is shown in FIG. 7, where “HW” represents the spectrum of the liquid composition; “+” represents positive quality result and “−” represents negative quality result. Two absorption peaks of BTB are shown in FIG. 7. These are peaks result in a yellow color for the more acidic case and green-blue when more basic. When added to liquid composition, a correlation between the color and the quality of the liquid composition was found. The green color (basic) of the liquid composition indicates higher quality.

### Example 5

### Zeta Potential Measurement

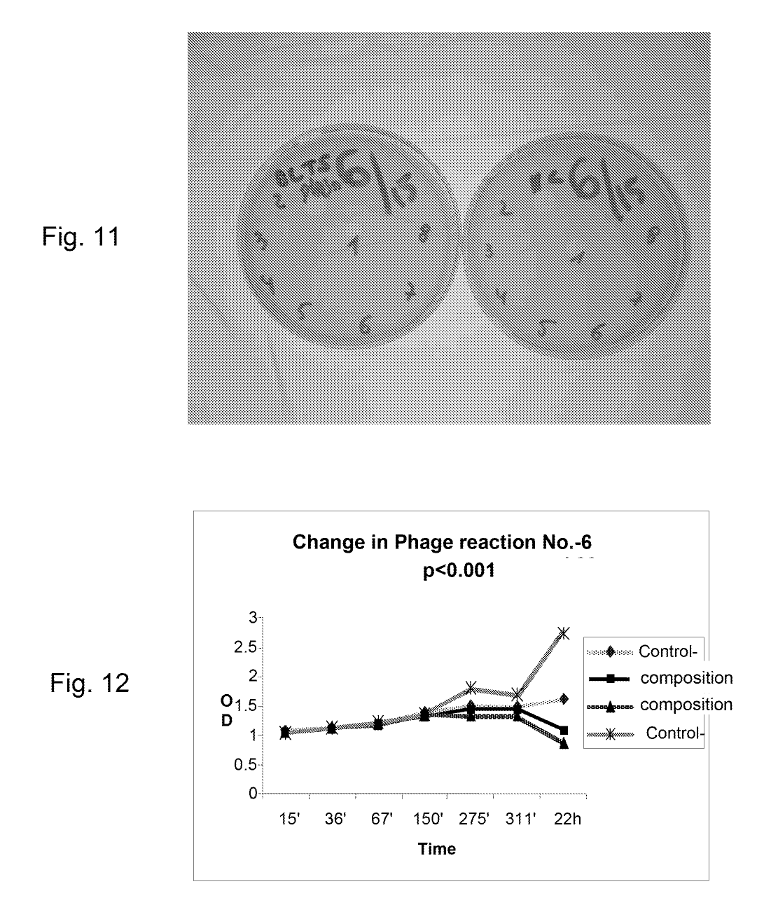

Zeta potential measurement was performed on the liquid composition of the present invention. FIG. 8 shows ζ potential of 6 samples: extra pure water, extra pure water shifted to pH 8, extra pure water shifted to pH 10, two samples of the liquid composition with positive quality and one sample of the liquid composition with negative quality. The measurement of the ζ potential was performed using a Zeta Sizer.

As shown, the ζ potential of the liquid composition of the present invention is significantly higher, indicating high mobility of the nanostructures in the liquid.

### Example 6

### Bacteriophage Reaction

The effect of the liquid composition of the present invention (LC9) on bacteriophage typing was investigated.

Materials and Methods

1) Bacteriophages No. 6 and 83A of a standard international kit for phage typing of staphylococcus aureus (SA), obtained from Public Health Laboratory In Colindale, UK, The International Reference Laboratory (URL: www.phls.co.uk), were examined.

2) Media for agar plates: Nutrient agar Oxoid No 2 (catalog number CM 67 Oxoid Ltd.)+CaCl₂. After autoclave sterilization 20 ml of CaCl₂ was added for each liter of medium.

3) Media for liquid cultures: Nutrient Broth No 2 Oxoid: 28 gr/1 liter.

4) Phage typing concentration: each bacteriophage was tested at 1 and 100 RTD (Routine Test Dilution).

5) Propagation of phage: each phage was propagated parallel on control and on tested media based on the liquid composition of the present invention.

6) The bacteriolysis surface area was measured using computerizes “Sketch” software for surface area measurements.

7) Statistical analysis: analysis-of-variance (ANOVA) with repeated measures was used for optic density analysis, and 2 ways ANOVA for lysis surface area measurements using SPSS™ software for Microsoft Windows™.

Results

Acceleration of Bacteriophage Reaction

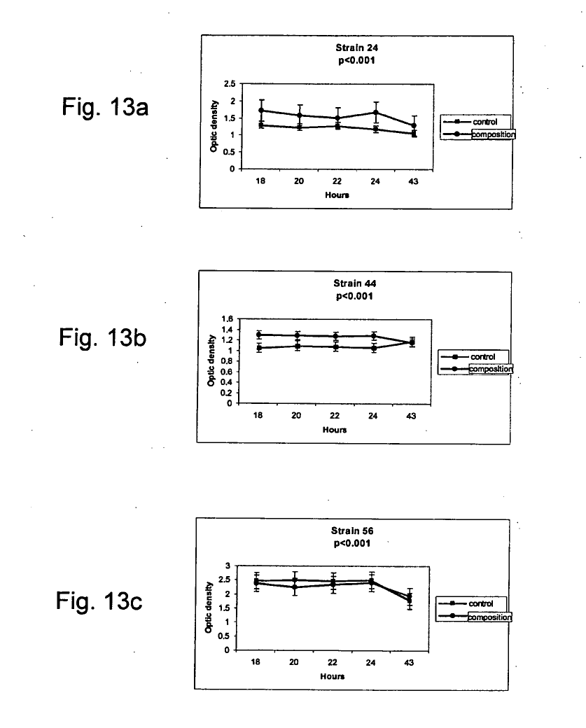

FIGS. 9a-b show the bacteriophage reaction on the tested media, as follows: FIG. 9a shows Bacteriophages No. 6 in a control medium (right hand side) and in the liquid composition of the present invention (left hand side); FIG. 9b shows Bacteriophages No. 83A in a control medium (right hand side) and in the liquid composition of the present invention. The bacteriophage reaction in the liquid composition of the present invention demonstrated an accelerated lysis of bacteria (within 1 hour in the liquid composition and 3 hours in the control media).

Superior lysis areas on the tested plates were observed immediately and remained larger at 24 hours of incubation. Vivid differences between the control and tested plates were demonstrated in the RTD concentrations.

Area Measurements

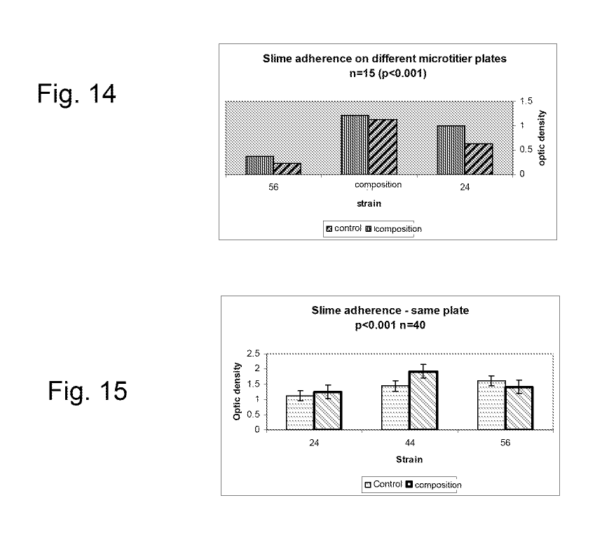

FIG. 10 is a histogram showing a comparison between the bacteriolysis surface areas of the control and liquid composition. Statistic significance was determined using 2 ways ANOVA for phage typing. The corresponding numbers are given in Tables 2 and 3, below.

TABLE 1

| Phage | Control | Composition |
|---|---|---|
| No. 6 | 2.488 | 6.084 |
| | 2.238 | 2.441 |
| | 3.246 | 5.121 |
| Average | 2.657333 | 4.548667 |
| STD | 0.524901 | 1.887733 |
| No. 83 | 2.898 | 7.369 |
| | 2.61 | 4.748 |
| | 4.692 | 8.261 |
| Average | 3.4 | 6.792667 |
| STD | 1.128133 | 1.826037 |

TABLE 2
2 way ANOVA - dependent variable: Area

| Factor | SS | d.f. | MS | F | Significance |
|---|---|---|---|---|---|
| Phage | 6.69 | 1 | 6.69 | 3.168 | 0.113 |
| Water | 20.94 | 1 | 20.94 | 9.917 | 0.014 |
| Phage-Water | 1.691 | 1 | 1.691 | 0.801 | 0.397 |

A significant increase in phage reaction area was found with the liquid composition (p=0.014). There no significant difference between the phages (p=0.113) and media interactions (p=0.397), which demonstrate that the liquid composition of the present invention has identical trends of effect on both tested phages.

RTD Determination

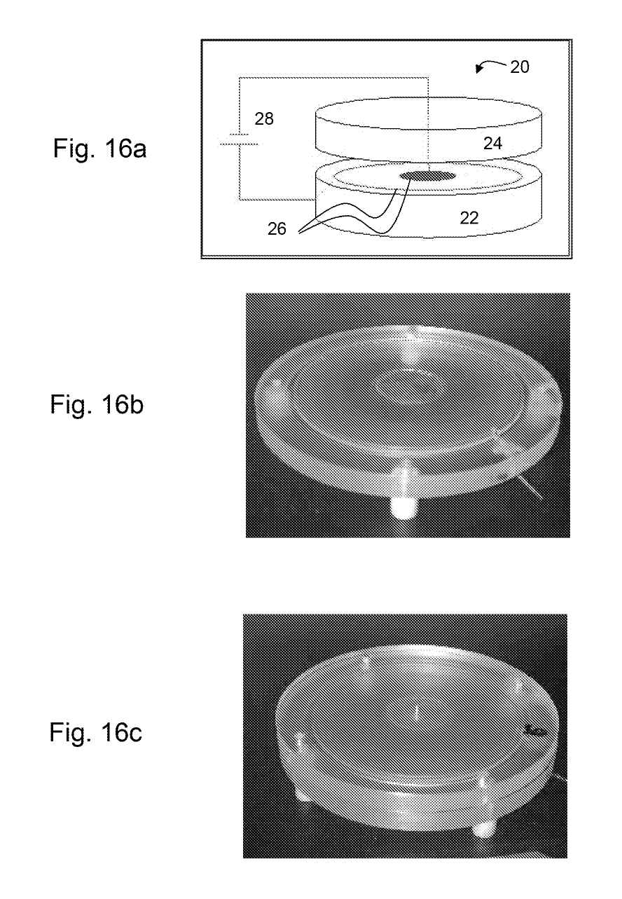

FIG. 11 shows increased dilution by 100 times in each increment. The increase concentration of phages in the liquid composition of the present invention by 100 times more was observed.

Bacteriolysis-Optic Density Reading

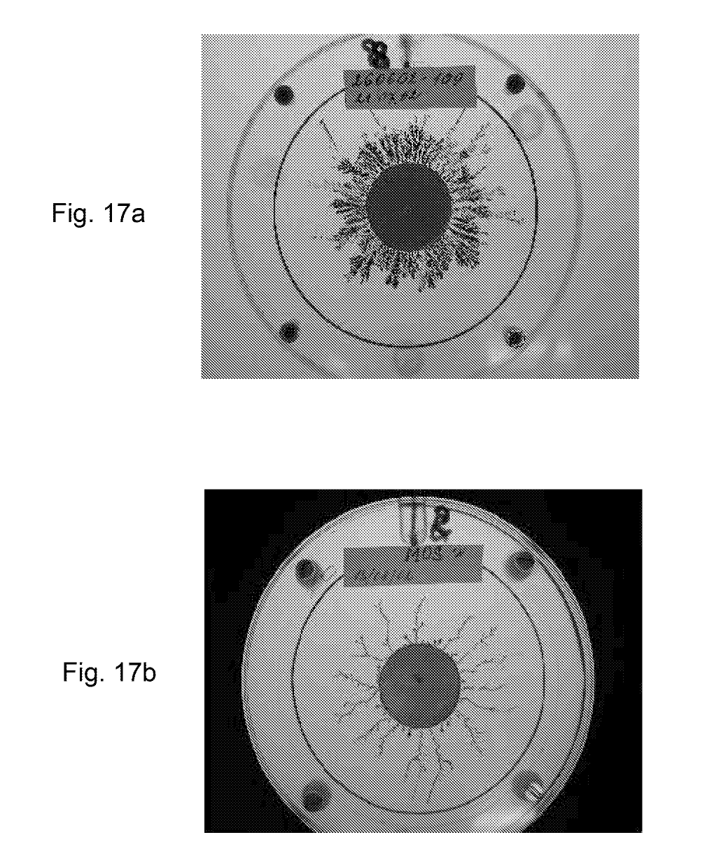

FIG. 12 is a graph of the optic density (OD) in phage No. 6, as a function of time. The corresponding numbers for mean change from start and the OD of phage reaction are given in Tables 3 and 4, respectively. The ANOVA for repeated measures is presented in Table 5.

TABLE 3

| | |
|---|---|
| phage No. 6 | phage No. 83A |

| Time | control | composition | control | composition |
|---|---|---|---|---|
| 15′ | 1.079109 | 1.052213 | 1.035938 | 1.038375 |
| 36′ | 1.142857 | 1.102157 | 1.139063 | 1.128668 |
| 67′ | 1.207373 | 1.205448 | 1.221875 | 1.180587 |
| 150′ | 1.407066 | 1.321226 | 1.366406 | 1.345372 |
| 275′ | 1.515361 | 1.434733 | 1.810938 | 1.3386 |
| 311′ | 1.483871 | 1.449489 | 1.686719 | 1.327314 |
| 22 h | 1.616743 | 1.094211 | 2.735938 | 0.87246 |

TABLE 4

| | phage No. 6 | | phage No. 83A | |
|---|---|---|---|---|
| Time | control | composition | control | composition |
| 0 | 0.668 | 0.446 | 0.642 | 0.428 |
| 0 | 0.634 | 0.435 | 0.638 | 0.458 |
| Average | 0.651 | 0.4405 | 0.64 | 0.443 |
| STD | 0.024042 | 0.007778 | 0.002828 | 0.021213 |
| 15 | 0.733 | 0.471 | 0.642 | 0.458 |
| 15 | 0.672 | 0.456 | 0.684 | 0.462 |
| Average | 0.7025 | 0.4635 | 0.687 | 0.46 |
| STD | 0.043134 | 0.010607 | 0.029698 | 0.002828 |
| 36 | 0.764 | 0.485 | 0.728 | 0.486 |
| 36 | 0.724 | 0.486 | 0.73 | 0.514 |
| Average | 0.744 | 0.4855 | 0.729 | 0.5 |
| STD | 0.028284 | 0.000707 | 0.001414 | 0.019799 |
| 67 | 0.799 | 0.537 | 0.777 | 0.523 |
| 67 | 0.773 | 0.525 | 0.787 | 0.523 |
| Average | 0.786 | 0.531 | 0.687 | 0.523 |
| STD | 0.018385 | 0.008485 | 0.007071 | 0 |
| 150 | 0.966 | 0.571 | 0.87 | 0.596 |
| 150 | 0.866 | 0.593 | 0.879 | 0.596 |
| Average | 0.916 | 0.582 | 0.8745 | 0.596 |
| STD | 0.070711 | 0.015556 | 0.006364 | 0 |
| 275 | 0.978 | 0.639 | 1.132 | 0.602 |
| 275 | 0.995 | 0.625 | 1.186 | 0.584 |
| Average | 0.9865 | 0.632 | 0.687 | 0.593 |
| STD | 0.012021 | 0.009899 | 0.038184 | 0.012728 |
| 311 | 0.964 | 0.644 | 1.081 | 0.602 |
| 311 | 0.968 | 0.633 | 1.078 | 0.574 |
| Average | 0.966 | 0.6385 | 1.0795 | 0.588 |
| STD | 0.002828 | 0.007778 | 0.002121 | 0.019799 |
| 22 h | 1.003 | 0.463 | 1.691 | 0.388 |
| 22 h | 1.102 | 0.501 | 1.811 | 0.385 |
| Average | 1.0525 | 0.482 | 0.687 | 0.3865 |
| STD | 0.070004 | 0.02687 | 0.084853 | 0.002121 |

TABLE 5

| Phage | Factor | SS | d.f. | MS | F | Significance |
|---|---|---|---|---|---|---|
| No. 83 | time | 17804.37 | 6 | 2967.396 | 164.069 | 0.001 |
| | time-water | 27350 | 6 | 4558.334 | 252.033 | 0.001 |
| | control- | 10851.38 | 1 | 10851.38 | 55.805 | 0.017 |
| | LC | | | | | |
| No. 6 | time | 6449.544 | 6 | 10.74.924 | 32.31 | 0.001 |
| | time-water | 2024.998 | 6 | 337.5 | 10.145 | 0.001 |
| | control- | 904.547 | 1 | 904.547 | 15.385 | 0.059 |
| | LC | | | | | |

As demonstrated in FIG. 12 and Tables 3-5, there is a significant correlation between the medium and the time. More specifically, there is a significant different trends in time between the control and the liquid composition of the present invention (p=0.001) both in phage No. 6 and in phage No. 83A. The phage reaction in the liquid composition of the present invention has significantly different trend with opposite direction.

At 22 hour an addition “kick” of lysis was observed which may be due to increased potency of the phage.

All the controls OD (media alone, phage alone, bacteria alone, in control and composition with different phages) demonstrated no difference between themselves and were significant different from tested reaction.

Conclusions

The liquid composition of the present invention accelerates the phage reaction time (×3); and increases the bacteriolysis surface area; increases the RTD (×100 or more)

The bacteriophage reactions in the liquid composition of the present invention demonstrate opposite trends compare to control in OD measurements, and increased potency with time.

Discussion

The kinetics of phage-host interaction has been enhanced in media containing the liquid composition. This was observed in repeated experiments and in measured “growth curve kinetics.” The parameters influencing the kinetics are independent of measured factors (e.g., pH, temperature, etc.) Not only phage concentration is increased but rather its potency, as was observed after 22 hours of reaction. Phages in control are already out of effect when phages in the liquid composition of the present invention are still effective. In addition, the propagating strains pre-treated with the liquid composition are much more effective.

### Example 7

### Effect of the Liquid Composition on Phage-Bacteria Interaction

The effect of the liquid composition of the present invention on Lambda (λ) phage was investigated. λ phage is used in molecular biology for representing the genome DNA of organisms. The following experiment relies on standard λ phage interaction applications. In all the experiments the materials in the test groups were prepared with the liquid composition as a solvent. The materials in control groups were prepared as described hereinbelow. The pH of the control groups was adjusted to the pH of the liquid composition solutions, which was between 7.2 and 7.4.

Materials and Methods

1) LB Medium

10 g. of Bacto Tryptone, 5 g of Yeast extract, 10 g of NaCl dissolved in 1000 ml of distilled water, and then sterilized by autoclave (121° C., 1.5 atm for 45 minutes). The plates were pre-incubated for two days before use.

2) LB Plates

15 g of Bacto Agar added to 1000 ml of LB medium, mixed and autoclaved as described above.

3) Top Agarose 0.7%

100 ml of LB medium mixed with 0.7 g of chemically pure, electrophoresis grade agarose (from Difco or other supplier), and then sterilized by autoclave (121° C., 1.5 atm during 45 minutes).

4) MgSO₄-10 mM

1.2 g of MgSO₄ was dissolved in 1000 ml distilled water and sterilized by autoclave.

5) Maltose 20% (w/v)

200 g of maltose was dissolved in 1000 ml distilled water, and sterilized by filtration through 20 μm filter.

6) MgSO₄-1 M

120.37 g of MgSO₄ was dissolved in 1000 ml distilled water and sterilized by autoclave.

7) LB with 10 mM of MgSO₄ and 0.2% of Maltose

100 μl of MgSO₄ 1M and 100 μl of maltose 20% added to 99.8 ml of LB medium.

8) SM Buffer (Phage Storage Buffer)

5.8 g of NaCl, 2 g of MgSO₄, 50 ml of 1M Tris HCl (pH 7.5), 5 ml of 2% (w/v) gelatin dissolved in distilled water, achieving the final volume of 1000 ml, and then, sterilized by autoclave.

9) Bacterial Strain (Host)

E. coli XL1 Blue MRA (Stratagene).

10) Phage:

λ GEM 11 (Promega).

11) Bacterial Cultivation on LB Plates

XL1 cells dispersed on the LB plate by bacteriological loop accordingly to common procedure of bacterial inoculation. The plates were incubated at 37° C. for 16 hours.

12) Bacterial Cultivation on LB Liquid Medium

Single colony of XL1 cells from LB plate inoculated in LB liquid medium with subsequent incubation at 37° C. for 16 hours (overnight), with shaking at 200 rpm.

13) Infection of the Host Bacterial Strain by the Phage

XL1 cells were inoculated into the LB medium supplemented with 10 mM of MgSO₄ and 0.2% of maltose. Incubation at 37° C. with shaking at 200 rpm continued, until turbidity of 0.6 at a wavelength of 600 nm was achieved (4-5 hours). The grown culture was centrifuged at 4000 rpm for 5 minutes. Supernatant was discarded, and the bacteria were re-suspended into the 10 mM of MgSO₄, until turbidity of 0.6 at wavelength of 600 nm was achieved. A required volume of SM buffer containing the phages was added into the 200 ml of the re-suspended bacteria. After the incubation at 37° C. for 15 minutes two alternatives were available:

(i) For lysate preparation an appropriate volume of LB medium was added to the host—phage mixture, and incubated at 37° C. for 16 hours (overnight), with shaking at 200 rpm.

(ii) For the phages appearance on the solid medium (plaques), a molten Top Agarose (50° C.) was poured to the host—phage mixture quickly mixed and spread on pre-warmed LB plate. After the agarose solidification, incubation was performed at 37° C. for 16 hours (overnight).

14) Extraction of the Phage DNA

Bacterial lysate was centrifuged at 6000 rpm for 5-10 minutes for sedimentation of the bacterial debris. Supernatant was collected and centrifuged at 14000 rpm for 30 minutes for sedimentation of the phage particles. Supernatant was discarded and the phage pellet was re-suspended in SM buffer without gelatin. A mixture of nucleases (RNase and DNase from any supplier) was added to the re-suspended phage for a final concentration of 5-10 Weiss units per 1 μl of the phage suspension. After an incubation of 30 minutes at 37° C., as required for complete digestion of any residual bacterial nucleic acids, the DNA of the phage was extracted by the following procedure:

(i) extraction with phenol: chloroform: iso-amil-alcohol (25:24:1 v/v);

(ii) removing of phenol contamination by chloroform;

(iii) precipitation to final concentration of 0.3 M Potassium Acetate and one volume of iso-propanol;

(iii) washing with 70% of ethanol; and

(iv) drying and re-suspension in distilled water for further analysis.

Results

Plaque Forming Unit (PFU) Titer Experiment.

Phage suspensions were prepared from phage stock in SM buffer in series of 1/10 dilutions: one in SM buffer based on liquid composition of the present invention and one in SM buffer based on ddH₂O.

1 μl of each dilution was incubated with 200 μl of competent bacterial host

(see methods, item 13). The mix was incubated at 37° C. for 15 minutes to allow the bacteriophage to inject its DNA into the host bacteria. After incubation a hot (45-50° C.) top agarose was added and the suspension was dispersed on the LB plate. Nine replications of each dilution and treatment were prepared.

Table 6 below presents the PFU levels which were counted after overnight incubation.

TABLE 6

| Phage | | |
|---|---|---|
| Dilution | Control | Composition |
| 10⁻³ | 506 | 724 |
| | 684 | 845 |
| | 761 | 704 |
| | 651 | 879 |
| | 618 | 617 |
| | 683 | 612 |
| | 932 | 860 |
| | 697 | 652 |
| | 746 | 891 |
| Average | 697.5556 | 753.7778 |
| S.D. | 115.6083 | 115.4597 |
| 10⁻⁴ | 70 | 119 |
| | 11 | 129 |
| | 77 | 90 |
| | 32 | 111 |
| | 96 | 91 |
| | 53 | 106 |
| | 56 | 120 |
| | 71 | 100 |
| | 25 | 183 |
| Average | 54.55556 | 116.5556 |
| S.D. | 27.41857 | 28.20067 |

The numbers were modified by square root transformation to normalize the data as required for performing parametrical tests. Table 7 below shows results of data analysis by factorial ANOVA.

TABLE 7

| Factors | SS | d.f. | MS | Significance |
|---|---|---|---|---|
| Treatment | 48.9147 | 1 | 48.9147 | P = 0.01 |
| Concentration | 2893.0255 | 1 | 2893.0255 | P = 0.01 |
| Interaction | 14.7506 | 1 | 14.7506 | P = 0.01 |
| Error | 239.8006 | 32 | 7.4938 | |

Significance levels: P 0.05 (d.f. 1; 32) = 4.14909, P 0.01 (d.f. 1; 32) = 7.49924.

Significant effect in the PFU titer was detected between concentrations (0.001 against 0.0001), treatment (test against control) and interactions (any combination of treatment and concentration). However, significant differences among concentrations were expected as a consequence of experiment structure. Significant increase in the PFU titer caused by the liquid composition of the present invention treatment requires special explanation, which is presented in the discussion section of this example, hereinbelow.

E. coli Strain XLI-Blue Bacterial Growth in LB.

2 μl of bacterial suspension were inoculated on each of 1/8 sector in two LB plates (16 inoculation totally), both in control and liquid composition of the present invention based media. After incubation at 37° C. during 3 days, colony shapes and sizes were observed. No significant differences were observed between control and the liquid composition treatments.

Phage Growth on LB Bacterial Culture (Lysate)

Lysate was prepared as described in methods (item 13), centrifuged to sediment bacterial debris and then turbidity was measured at 600 nm. Later, DNA was extracted from lysates as it is described hereinabove in the methods (item 14). No significant differences were observed between control and the liquid composition treatments both in turbidity and extracted DNA concentration (0.726 μg/μl in control; 0.718 μg/μl in the liquid composition).

Discussion

In two independent tests out of three, a significant increase in PFU at low phage dilutions (10⁻³ and 10⁻⁴) was observed, when the liquid composition of the present invention was used compared to the control.

The probable explanation to the above observation lies in the fact that plaques formation depend on two separate processes: phage infecting their hosts (infectivity) and on host compatibility to the phage.

The host compatibility depends on the ability of the phage using bacterial mechanisms for phage reproduction. No correlation between the liquid composition of the present invention to the host compatibility was found. In case of increased compatibility the plaques have to be larger than the control (larger distance from the initial infection site), or the phage particles number must be greater than the control.

The fact that the liquid composition of the present invention did not affect DNA phage level supports the previous founding.

The infectivity depends on essential phage particles and/or bacterial cells capability to be infected by the phage. The significant increase in PFU when the liquid composition of the present invention was used (about 2-fold greater than the control) indicates that the liquid composition of the present invention affects the infectivity. Pre-infection treatments (see methods, item 13), are required for increasing probability of infection by preparing competent bacteria, which are easier infected by phage than non-treated bacteria.

At low phage dilutions the limiting factor of the PFU formation is the host cell ability to be infected by the phage.

It seems that bacteria that was treated and grown with the liquid composition of the present invention had the capability to be infected by the phage more than control bacteria. It is therefore assumed that the liquid composition increases affinity between bacterial receptor and phage particles.

### Example 8

### Effect of the Liquid Composition on the Adherence of Coagulase-Negative Staphylococci to Microtiter Plate

Production of slime polysaccharide, is crucial to biofilm generation and maintenance, and plays a major part as a virulence factor in bacteria [Gotz F., “Staphylococcus and biofilms,” Mol Microbiol 2002, 43(6):1367-78]. The slime facilitates adherence of bacteria to a surface and their accumulation to form multi-layered clusters. Slime also protected against the host's immune defense and antibiotic treatment [Kolari M. et al., “Colored moderately thermophilic bacteria in paper-machine biofilms,” to apear in J Ind Microbiol Biotechnol 2003]. Biofilm produced by bacteria can cause problems also in industry.

Most of current concepts for the prevention of slime are associated with search for new anti-infective active in biofilm and new biocompatible materials that complicate biofilm.

It has been demonstrated [Besnier J M et al., “Effect of subinhibitory concentrations of antimicrobial agents on adherence to silicone and hydrophobicity of coagulase-negative staphylococci,” Clin Microbiol Infect 1996, 1(4):244-248] that the adherence of coagulase-negative staphylococci onto silicone can be modified by sub-MICs of antimicrobial agents. This effect was different in the slime-producing and non-slime-producing strains, and was not correlated with the mechanism of the inhibitory effect of these antimicrobial agents, or the modification of hydrophobicity suggesting that some surface components, not involved in hydrophobicity, could play a role in vitro adherence.

The bacterial resistance of Staphylococcus epidermidis, a serious pathogen of implant-related infections, to antibiotics is related to the production of a glycocalyx slime that impairs antibiotic access and the killing by host defense mechanisms [Konig D P et al., “In vitro adherence and accumulation of Staphylococcus epidermidis RP 62 A and Staphylococcus epidermidis M7 on four different bone cements,” Langenbecks Arch Surg 2001, 386(5):328-32]. In vitro studies of different bone cements containing antibiotics, developed for the prevention of biomaterial-associated infection, could not always demonstrate complete eradication of biomaterial-adherent bacteria. Further efforts are done to find better protection from slime adherence.

In addition, surface interaction can modify slime adherence. For example, Farooq et al. [Farooq M et al., “Gelatin-sealed polyester resists Staphylococcus epidermidis biofilm infection,” J Surg Res 1999, 87(1):57-61] demonstrated that gelatin-impregnated polyester grafts inhibit Staphylococcus epidermidis biofilm infection in a canine model of aortic graft interposition. Gelatin-impregnated polyester grafts demonstrated in vivo resistance to coagulase-negative staphylococcal biofilm infection.

The objectives of the experiments in this example were to investigate the effect of the liquid composition of the present invention on the adherence to plastic of a slime-producing Staphylococcus epidermidis (API-6706112)

Methods

The bacteria used were identified using Bio Merieux sa Marcy l'Eoile, France (API) with 98.4% confidence for Staphylococcus epidermidis 6706112. Table 8, below summarizes the three bacterial strains which were used.

TABLE 8

| Bacterial strain | API No. | Confidence |
|---|---|---|
| 24 | 6706112 | 98.4% |
| 44 | 6706112 | 98.4% |
| 56 | 6706112 | 98.4% |

Slime adherence was quantitatively examined with spectrophotometer optical density (OD) technique, as follows. Overnight cultures in TSB with the liquid composition of the present invention and with regular water were diluted 1:2.5 with corresponding media and placed in sterile micro titer tissue culture plates (Cellstar, Greniner labortechnik, Tissue culture plate, 96W Flat bottom, with LID, sterile No. 655180) for total volume of 250 μl each for incubation in 37° C. The plates were rinsed 3 times with tape water, stained with crystal violet, and rinsed 3 times with tape water. After drying, the OD of the stained adherent bacterial films were measured with a MicroElisa Auto reader (MR5000; Dynatech Laboratories, Alexandria Va.) by using wavelength of 550 nm. OD of bacterial culture was measured before each staining using dual filter of 450 nm and 630 nm. The test of each bacterial strain was performed in quadruplicates.

The experiment was design to evaluate slime adherence in intervals. The time table for the kinetics assessment was 18, 20, 22, 24 and 43 hours. All three (3) strains were evaluated on the same plate. The liquid composition was used for standard media preparation and underwent standard autoclave sterilization.

Adherence values were compared using ANOVA with repeated measures for the same plate examination; grouping factors were plate and strain. Three-way ANOVA was used for the different plate examination using SPSS™ 11.0 for Microsoft Windows™.

Results

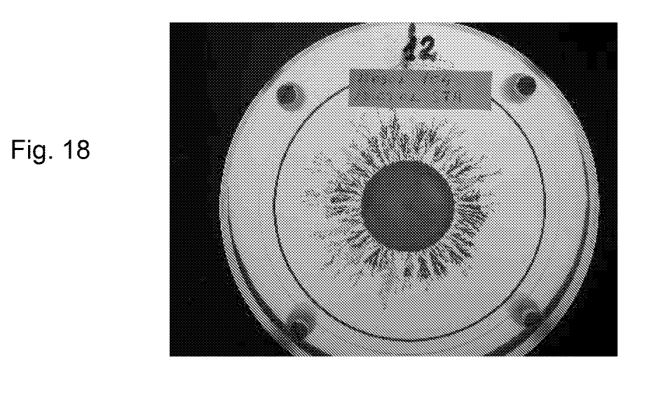

FIGS. 13a-c show the OD in all the slime-producing Staphylococcus epidermidis (see Table 8, above). Adherence was significantly different (p<0.001) in the liquid composition of the present invention.

The kinetics of Strains 24 and 44 demonstrated increased slime adherence (FIGS. 13a-b, respectively) and strain 56 demonstrated decreased adherences (FIG. 13c). Time was found to be a significant factor in decreasing adherence where in the last hour the lowest adherences were observed. Significant differences were found between the stains (p<0.001), each strain has its own adherence characteristics. Significant interaction was found between the different strains and time (p<0.001), the differences between the strains is time dependent. Regression analysis found no interaction between time and type of water used (p=0.787), the differences between the adherences in the liquid composition and in the control maintained at all times, beginning to be apparent at the 18th hour and greatest effect at the 43rd hour.

Significant interaction between the strains and water (p<0.001) was found, the differences between the liquid composition and the control water was strain dependent. Each strain had its own adherence characteristics.

No interaction was found between strains time and water (p=0.539).

Table 9, below summarizes the results of Slime adherence kinetics (Three-way ANOVA).

TABLE 9

| Factor | SS | df | MS | F | Significance |
|---|---|---|---|---|---|
| Time | 1.356 | 4 | 0.339 | 8.624 | 0.001 |
| Strain | 28.285 | 2 | 14.143 | 359.743 | 0.001 |
| Water | 0.731 | 1 | 0.731 | 18.599 | 0.001 |
| Time-Strain | 1.072 | 8 | 0.134 | 3.41 | 0.002 |
| Time-water | 6.75E−02 | 4 | 1.69E−02 | 0.429 | 0.787 |
| Strain-Water | 1.052 | 2 | 0.526 | 13.374 | 0.001 |
| Time-Strain- | 0.276 | 8 | 3.45E−02 | 0.877 | 0.539 |
| water | | | | | |

Repeated experiments of slime adherence were performed at 24 hours of incubation on different plates from the same type, where each strain was incubated in a different micro titer plate.

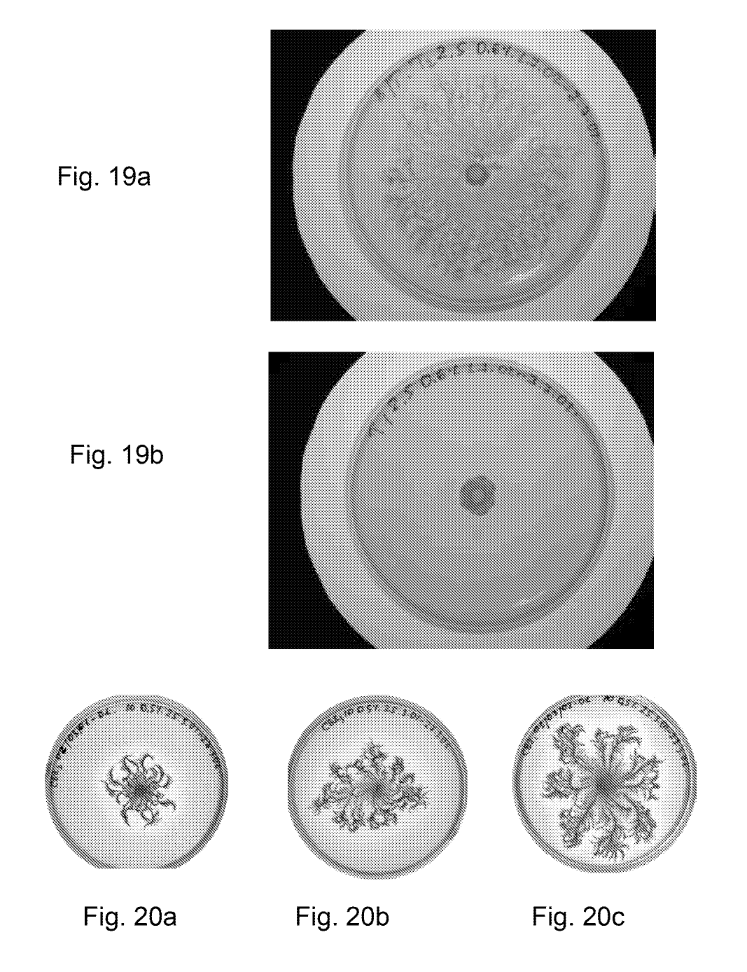

FIG. 14 is a histogram representing 15 repeated experiments of slime adherence on different micro titer plates. As shown, the adherence in the presence of the liquid composition is higher than the adherence in the control.

Significant adherence differences in the liquid composition and control, between the micro titer plates, and, among the strains were found (p<0.001). Significant interactions were found between plats, strain and water used. The extent of adherence is dependent on the strain, on the plat, and, on the water used.

Table 10, below summarizes the results of slime adherence on different micro titer plats (Three-way ANOVA).

TABLE 10

| Factor | SS | df | MS | F | Significance |
|---|---|---|---|---|---|
| Plate | 0.572 | 2 | 0.286 | 29.798 | 0.001 |
| Strain | 9.484 | 2 | 4.742 | 494.346 | 0.001 |
| Water | 1.288 | 1 | 1.288 | 134.276 | 0.001 |
| Plate-Strain | 1.265 | 4 | 0.316 | 32.976 | 0.001 |
| Plate-water | 2.15E−01 | 2 | 1.07E−01 | 11.183 | 0.001 |
| Strain-Water | 0.288 | 2 | 0.144 | 15.021 | 0.001 |
| Plate-Strain-water | 0.259 | 4 | 6.47E−02 | 6.744 | 0.001 |

To examine the possibility of plate to plate variation, multiple analyses were performed on the same plate (all strains).

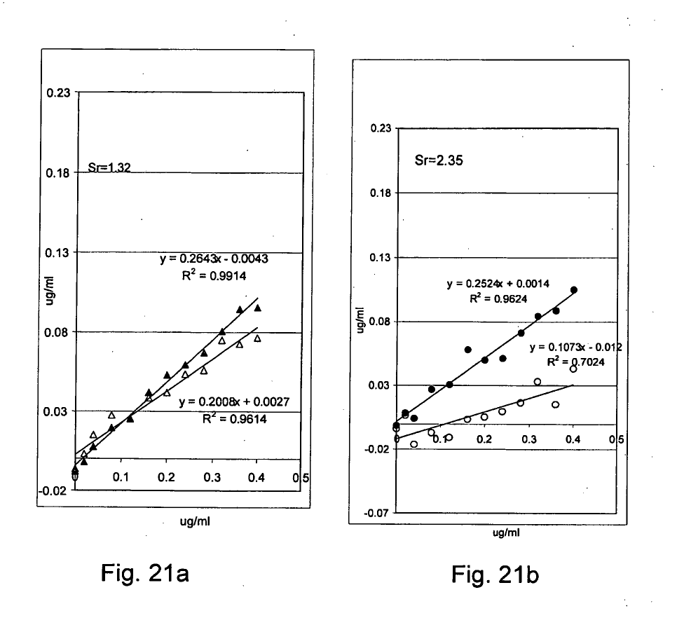

FIG. 15 shows slime adherence differences in the liquid composition of the present invention and the control on the same micro titer plate. Tables 11-12, below summarize the results of slime adherence on the same micro titer plat (ANOVA with repeated measurements).

As shown in Tables 11-12, significant difference between slime adherence with the liquid composition and Control once more was confirmed. However, new significant interactions between plate (p<0.001), strain (p<0.001), and water (p<0.001) were also found, confirming that the adherence differences in the liquid composition depend also on the plate, strain and interactions therebetween.

A significance difference in adherence between the strains and the plate points out the possibility of plate to plate variations. The above farther interactions in adherence open out a range of variation which is a result of the liquid composition. Plate to plate variation with the liquid composition indicates that there may be other factors on the plate surface or during plate preparation which could interact with the liquid composition.

TABLE 11

| Factor | SS | df | MS | F | Significance |
|---|---|---|---|---|---|
| Plate | 3.726 | 2 | 1.863 | 40.32 | 0.001 |
| Strain | 8.93 | 2 | 4.465 | 96.623 | 0.001 |
| Plate-Strain | 1.019 | 4 | 0.255 | 5.515 | 0.001 |

TABLE 12

| Factor-within subjects effects | F | Significance |
|---|---|---|
| composition-control | 17.106 | 0.001 |
| composition-control-plate | 6.496 | 0.001 |
| composition-control-strain | 50.165 | 0.001 |
| composition-control-plate-strain | 0.896 | 0.001 |

Discussion

The ability of the liquid composition of the present invention to change the surface interaction of the water, thereby to change bacterial adherence was studied. The media with the liquid composition contained the same buffering ability and underwent same autoclave sterilization, ruling out any organic or PH modification. Hydrophocity modification in the liquid composition can lead to environmental preference for the slime to be less or more adherence. The change in surface characteristics may be explained by a new order, which is introduced by the nanostructures, leading to a change in water hydrophobic ability.

### Example 9

### Electrochemical Deposition Tests

The liquid composition of the present invention has been subjected to a series of electrochemical deposition tests, in a quasi-two-dimensional cell.

Experimental Setup

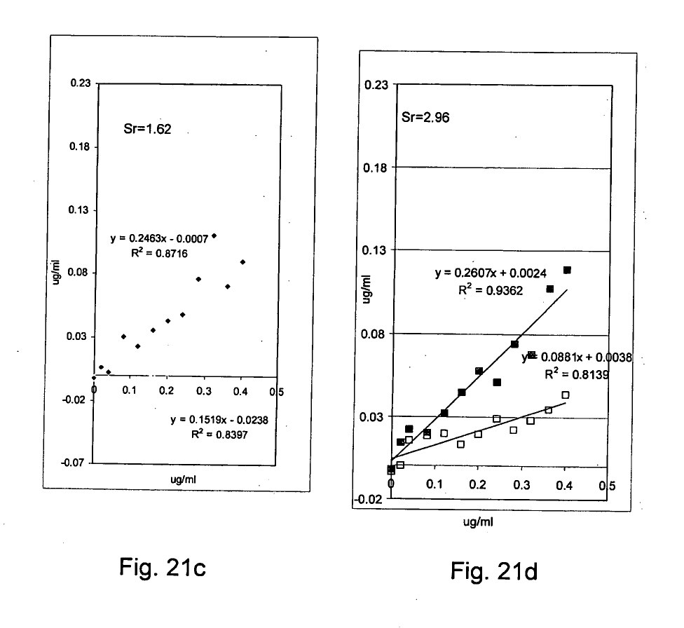

The experimental setup is shown in FIGS. 16a-c. A quasi-two-dimensional cell 20, 125 mm in diameter, included a Plexiglas base 22 and a Plexiglas cover 24. When cover 24 was positioned on base 22 a quasi-two-dimensional cavity, about 1 mm in height, was formed. Two concentric electrodes 26 were positioned in cell 20 and connected to a voltage source 28 of 12.4±0.1 V. The external electrode was shaped as a ring, 90 mm in diameter, and made of a 0.5 mm copper wire. The internal electrode was shaped as a disc having a thickness of 0.1 mm and diameter of 28 mm. The external electrode was connected to the positive pole of the voltage source and the internal electrode was connected to the negative pole thereof.

First, the experimental setup was used to perform electrochemical deposition process directly on the liquid composition of the present invention and, for comparison, on a control solution composed of Reverse Osmosis (RO) water.

Second, the experimental setup was used to examine the capability of the liquid composition to leave an electrochemical deposition signature, as follows. The liquid composition was placed in cell 20. After being in contact with base 22 for a period of 30 minutes, the liquid composition was replaced with RO water and an electrochemical deposition process was performed on the RO water.

Results

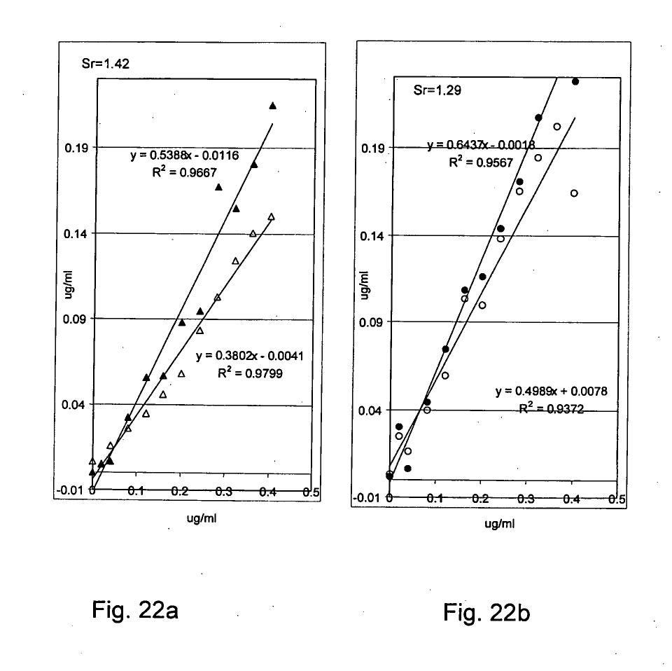

FIGS. 17a-b show electrochemical deposition of the liquid composition of the present invention (FIG. 17a) and the control (FIG. 17b). A transition between dense branching morphology and dendritic growth were observed in the liquid composition. The dense branching morphology spanned over a distance of several millimeters from the face of the negative electrode. In the control, the dense branching morphology was observed only in close proximity to the negative electrode and no morphology transition was observed.

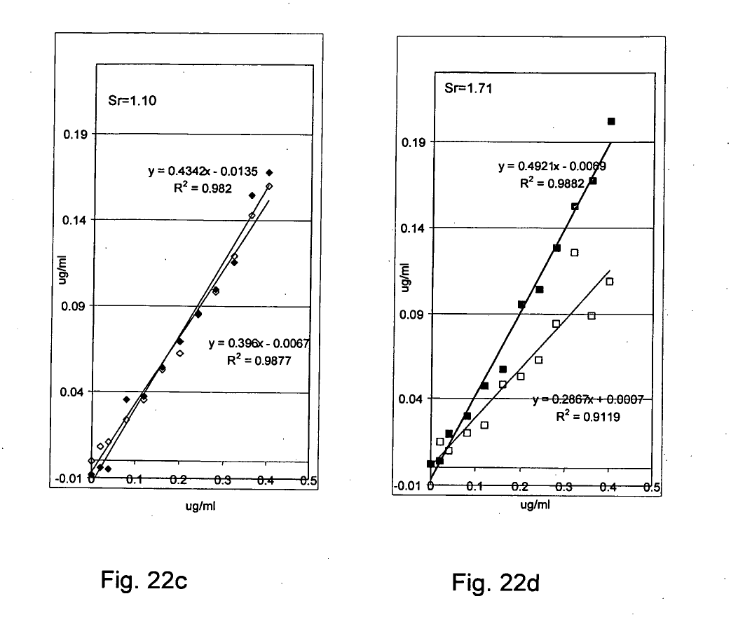

FIG. 18 shows electrochemical deposition of RO water in a cell, which was in contact with the liquid composition of the present invention for a period of 30 minutes. Comparing FIGS. 18 and 17b, one can see that the liquid composition leaves a clear signature on the surface of the cell, hence allows the formation of the branching and dendritic morphologies thereon. Such formation is absent in FIG. 17b where the RO water was placed on a clean cell.

The capability of the liquid composition to preserve an electrochemical deposition signature on the cell can be explained as a long range order which is induced on the RO water by the cell surface after the incubation with the liquid composition.

### Example 10

### Bacterial Colonies Growth

Colony growth of Bacillus subtilis was investigated in the presence of the liquid composition of the present invention. The control group included the same bacteria in the presence of RO water.

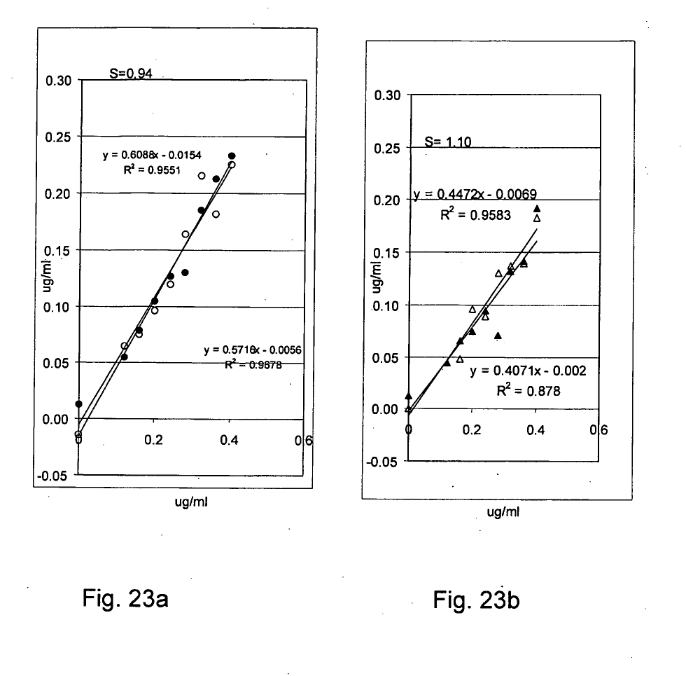

FIGS. 19a-b show results of Bacillus subtilis colony growth, for the liquid composition (FIG. 19a) and the control (FIG. 19b). As shown, the liquid composition of the present invention significantly accelerates the colony growth.

To further demonstrate the unique feature of the liquid composition of the present invention, an additional experiment was performed using a mixture of the raw powder, from which the nanostructure of the liquid composition are formed, and RO water, without the manufacturing process as further detailed above. This mixture is referred to hereinafter as Source Powder (SP) water.

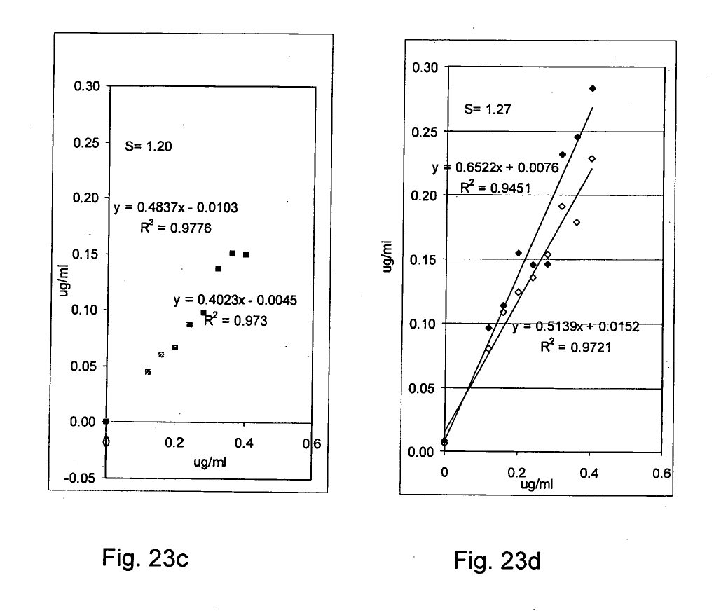

FIGS. 20a-c show the results of Bacillus subtilis colony growth, for the SP water (FIG. 20a), RO water (FIG. 20b) and the liquid composition (FIG. 20c). As shown, the colony growth in the presence of the SP water is even slower than the colony growth in the RO water, indicating that the raw material per se has a negative effect on the bacteria. On the other hand, the liquid composition of the present invention significantly accelerates the colony growth, although, in principle, the liquid composition is composed of the same material.

### Example 11

### Macromolecule Binding to Solid Phase Matrix

A myriad of biological treatments and reactions are performed on solid phase matrices such as Microtitration plates, membranes, beads, chips and the like. Solid phase matrices may have different physical and chemical properties, including, for example, hydrophobic properties, hydrophilic properties, electrical (e.g., charged, polar) properties and affinity properties.

The objectives of the experiments described in this example were to investigate the effect of the liquid composition of the present invention on the binding of biological material to microtitration plates and membranes having different physical and chemical properties.

Methods

The following microtitration plates, all produced by NUNC™ were used: (i) MaxiSorp™, which contains mixed hydrophilic/hydrophobic regions and is characterized by high binding capacity of and affinity for IgG and other molecules (binding capacity of IgG equals 650 ng/cm²); (ii) PolySorp™, which has a hydrophobic surface and is characterized by high binding capacity of and affinity for lipids; (iii) MedimSorp™, which has a surface chemistry between PolySorp™ and MaxiSorp™, and is characterized by high binding capacity of and affinity for proteins; (iv) Non-Sorp™, which is non-treated microtitration plate characterized by low binding capacity of and affinity for biomolecules; and (v) MultiSor™, which has a hydrophilic surface and is characterized by high binding capacity of and affinity for Glycans.

The following microtitration plates of CORNING™ (Costar) were used: (i) a medium binding microtitration plate, which has a hydrophilic surface and a binding capacity to IgG of 250 ng/cm²; (ii) a carbon binding microtitration plate, which covalently couples to carbohydrates; (iii) a high binding microtitration plate, which has a high adsorption capacity; and (iv) a high binding black microtitration plate, also having high adsorption capacity.

The binding efficiency of bio-molecules to the above microtitration plates was tested in four categories: ionic strengths, buffer pH, temperature and time.

The binding experiments were conducted by coating the microtitration plate by fluorescent-labeled bio-molecules or with a mixture of labeled and non-labeled bio-molecules of the same type, removal of the non-bound molecules by washing and measuring the remaining fluorescent signal on the plate.

The following protocol was employed:

1) Pre-diluting the fluorescent labeled bio-molecule to different concentration (typically 0.4-0.02 μg/ml) in a binding buffer. Each set of dilution was performed in two binding buffers: (i) the liquid composition of the present invention; and (ii) control RO water.

2) Dispensing (in triplicates) 100 μl samples from each concentration to the microtitration plate, and measuring the initial fluorescence level.

3) Incubating the plates overnight at 4° C. or 2 hours at 37° C.

4) Discarding the coating solution.

5) Adding 150 μl of washing solution to each well and agitate at room temperature for 5 minutes. This washing step was repeated three times. Typical washing solution includes 1× PBS, pH 7.4; 0.05% Tween20™; and 0.06 M NaCl.

6) Adding 200 μl fluorescence reading solution including 0.01 M NaOH and incubating for 180 minutes or overnight at room temperature.

7) Reading the fluorescence using a fluorescence bottom mode, with excitation wavelength of 485 nm, emission wavelength of 535 and optimal gain of 10 flashes.

The effect of the liquid composition of the present invention on the biding efficiency of glycoproteins (IgG of 150,000 D either labeled or non-labeled) to the above described plates was investigated. IgG is a polyclonal antibody composed of a mixture of mainly hydrophilic molecules. The molecules have a carbohydrate hydrophilic region, at the universal region and are slightly hydrophobic at the variable region. Such types of molecules are known to bind to MaxiSorp™ plates with very high efficiency (650 ng/cm²).

The following types of liquid composition of the present invention were used: LC1, LC2, LC3, LC4, LC5 and LC6, as further detailed hereinabove.

Table 13 below summarizes six assays which were conducted for IgG. In Table 13, assays in which only labeled antibodies were used are designated Ab*, and assays in which a mixture of labeled and non-labeled antibodies were used are designated Ab*/Ab.

TABLE 13

| | | Coating | | | Read |
|---|---|---|---|---|---|
| Assay | Plate type | condition | Washing buffer | Reading buffer | time |
| Ab*/Ab | Medium | 0.05M | 0.1M phosphate | 0.01M NaOH | 120′ |
| Ab* | (Costar) | carbonate buffer | buffer + 0.2M NaCl + | LC1 | |
| | | LC1 | 0.05% tween | C-5(1C) | |
| | | C-lot | LC1 | | |
| | | 5(1C) | C-5(1C) | | |
| | | O.N. 4° C. | | | |
| Ab*/Ab | Medium | 0.05M | 0.1M phosphate | 0.01M NaOH | 120′ |
| Ab* | (Costar) | Carbonate buffer | buffer + 0.2M NaCl + | LC1 | |
| | | LC1 | 0.05% tween | C-5(1C) | |
| | | C-5(1C) | LC1 | | |
| | | O.N. 4° C. | C-5(1C) | | |
| | | | 1xPBS + 0.06M NaCl + | | |
| | | | 0.05% tween | | |
| | | | LC2 | | |
| | | | C-(2C) | | |
| Ab*/Ab | Medium | 0.05M | 1xPBS + 0.06M NaCl + | 0.01M NaOH | 120′ |
| Ab* | (Costar) | Carbonate buffer | 0.05% tween | LC1 | |
| | Polysorp | LC1 | LC2 | C-5(1C) | |
| | Maxisorp | C-5(1C) | C-(2C) | | |
| | Non-sorp | O.N. 4° C./ | | | |
| | | RT O.N. | | | |
| Ab*/Ab | Medium | 0.05M | 1xPBS + 0.06M NaCl + | 0.01M NaOH | 120′ |
| Ab* | (Costar) | Carbonate buffer | 0.05% tween | LC5 | |
| | Polysorp | LC3 | LC5 | C-5(1C) | |
| | Maxisorp | C-(2C) | C-(2C) | | |
| | Non-sorp | O.N. 4° C. | | | |
| Ab*/Ab | Black | 0.05M Carbonate | 1xPBS + 0.06M NaCl + | 0.01M NaOH | 120′ |
| Ab* | (Costar) | buffer | 0.05% tween | C-(2C) | |
| | White | C-(2C) | C-(2C) | | |
| | (Costar) | O.N. 4° C. | | | |
| | Transparent | | | | |
| | (Costar) | | | | |
| Ab* | Medium | 0.05M Carbonate | 1xPBS + 0.06M NaCl + | 0.01M NaOH | 120′ |
| | (Costar) | buffer | 0.05% tween | LC3 | |
| | Polysorp | LC3 | LC4 | C-3C-5C | |
| | Maxisorp | C-2C | C-(2C) | | |
| | Non-sorp | O.N. 4° C./2 h 37 C.° | | | |

The effect of the liquid composition of the present invention on the binding efficiency of Peanut (Arachis hypogaea) agglutinin (PNA) was investigated on the MaxiSorp™ and Non-Sorp™ plates. PNA is an 110,000 Daltons lectin, composed of four identical glycoprotein subunits of approximately 27,000 Daltons each. PNA lectin binds glycoproteins and glycolipids with a specific configuration of sugar residues through hydrophilic regions. PNA also possesses hydrophobic regions. The assay, designated PNA*, included the use of three coating buffers: (i) carbonate buffer, pH 9.6, (ii) acetate buffer, pH 4.6 and (iii) phosphate buffer, pH 7.4. Table 14, below summarizes the experiment.

TABLE 14

| | Plate | Coating | | | Read |
|---|---|---|---|---|---|
| Assay | type | condition | Washing buffer | Reading buffer | time |
| PNA* | Maxisorp | 0.05M | 1xPBS + 0.06M NaCl + | 0.01M NaOH | 120′ |
| | Non-sorp | Carbonate | 0.05% tween | LC3 | |
| | | buffer | LC4 | C-3C-5C | |
| | | LC6 | C-(2C) | | |
| | | C-(2C) | | | |
| | | 0.1M | | | |
| | | acetate buffer | | | |
| | | LC6 | | | |
| | | C-(2C) | | | |
| | | 0.1M phosphat | | | |
| | | buffer | | | |
| | | LC1 | | | |
| | | C-5(1C) | | | |
| | | O.N. 4° C. | | | |

The effect of the liquid composition of the present invention on binding efficiency of nucleic acid was investigated on the MaxiSorp™, Polysorp™ and Non-Sorp™ plates. Generally, DNA molecules do not bind easily to polystyrene plates. Even more problematic is the binding of oligonucleotides, which are small single strand DNA, having a molecular weight of several thousands Dalton. Table 15 below summarizes the experiments which were conducted for labeled oligonucleotide binding. The assays are designated by Oligo*.

TABLE 15

| | Plate | Coating | | | Read |
|---|---|---|---|---|---|
| Assay | type | condition | Washing buffer | Reading buffer | time |
| Oligo* | Maxisorp | 0.05M | 1xPBS + 0.06M NaCl + | 0.01M NaOH | 180′ |
| | Non-sorp | Carbonate | 0.05% tween | LC3 | |
| | | buffer | LC4 | C-3C-5C | |
| | | LC6 | C-(2C) | | |
| | | C-(2C) | | | |
| | | 0.1M | | | |
| | | acetate buffer | | | |
| | | LC6 | | | |
| | | C-(2C) | | | |
| | | 0.1M | | | |
| | | phosphat | | | |
| | | buffer LC1 | | | |
| | | C-5(1C) | | | |
| | | 2 h 37° C. | | | |
| Oligo* | Polysorp | 0.05M | 1xPBS + 0.06 | 0.01M NaOH | 180′ |
| | Maxisorp | Carbonate | M NaCl + 0.05% | LC3 | |
| | | buffer | tween | C-3C-5C | |
| | | LC6 | LC2 | | |
| | | C-(2C) | C-(2C) | | |
| | | 0.1M | | | |
| | | acetate buffer | | | |
| | | LC6 | | | |
| | | C-(2C) | | | |
| | | 0.1Mphosphat | | | |
| | | buffer | | | |
| | | LC1 | | | |
| | | C-5(1C) | | | |
| | | O.N. 4° C. | | | |
| Oligo* | Polysorp | 0.1M | 1xPBS + 0.06M NaCl + | 0.01M NaOH | 180′ |
| | Maxisorp | acetate buffer + | 0.05% tween | LC3 | |
| | | 0.2M | LC4 | C-3C-5C | |
| | | sodium | C-(2C) | | |
| | | acetate | | | |
| | | LC6 | | | |
| | | C-(2C) | | | |
| | | 0.1M | | | |
| | | phosphate | | | |
| | | buffer + | | | |
| | | 0.2M | | | |
| | | sodium | | | |
| | | acetate | | | |
| | | LC1 | | | |
| | | C-5(1C) | | | |
| | | 2 h 37° C. | | | |

IgG Results and Discussion

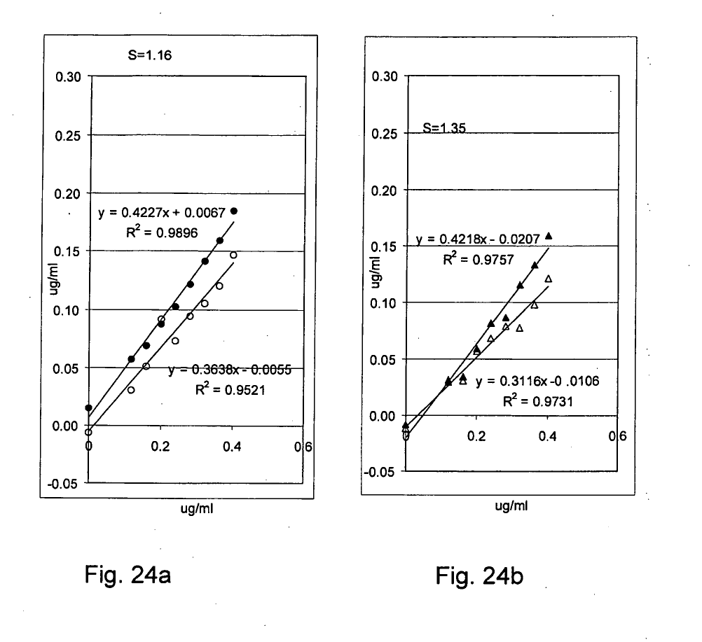

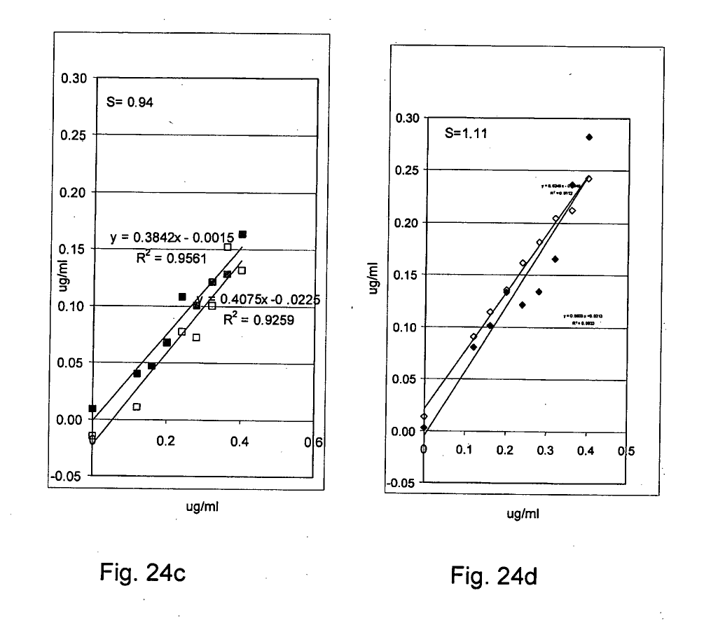

FIGS. 21a-22d show the results of the Ab*/Ab assays (FIGS. 21a-d) and the Ab* assays (FIGS. 22a-d) to the medium Costar™ (a), Non-SorpT™ (b), Maxisorp™ (c) and Polysorp™ (d) plates. The results obtained using the liquid composition of the present invention are marked with full symbols (triangles, squares, etc.) and the control results are marked with empty symbols. The lines correspond to linear regression fits. The binding efficiency can be estimated by the slope of the lines, whereby a larger slope corresponds to a better binding efficiency.

As shown in FIGS. 21a-22d, the slopes obtained using the liquid composition of the present invention are steeper than the slopes obtained in the control experiments. Thus, the liquid composition of the present invention is capable of enhancing the binding efficiency. The enhancement binding capability of the liquid composition of the present invention, is designated Sr and defined as the ratio of the two slopes in each Figure, such that Sr>1 corresponds to binding enhancement and Sr<1 corresponds to binding suppression. Hence, the values of the Sr parameter calculated for the slops obtained at FIGS. 21a-d were, 1.32, 2.35, 1.62 and 2.96, respectively, and the values of the Sr parameter calculated for the slops obtained at FIGS. 22a-d were, 1.42, 1.29, 1.10 and 1.71, respectively.

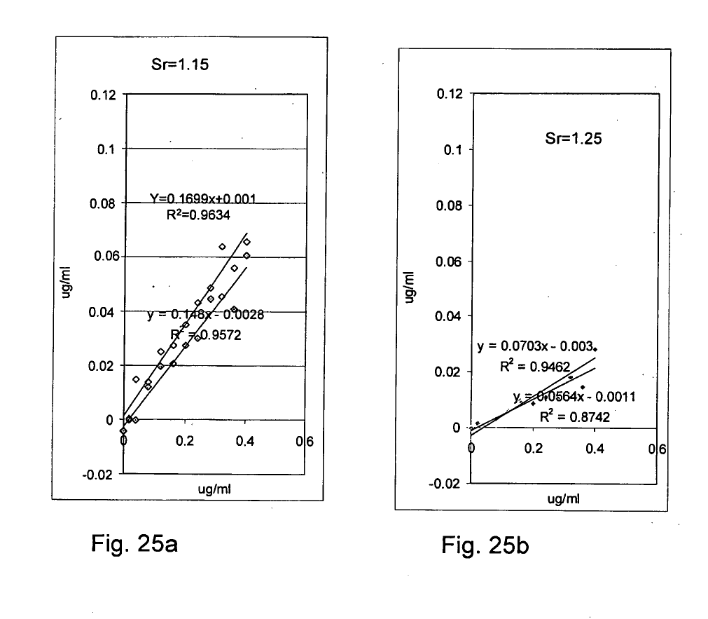

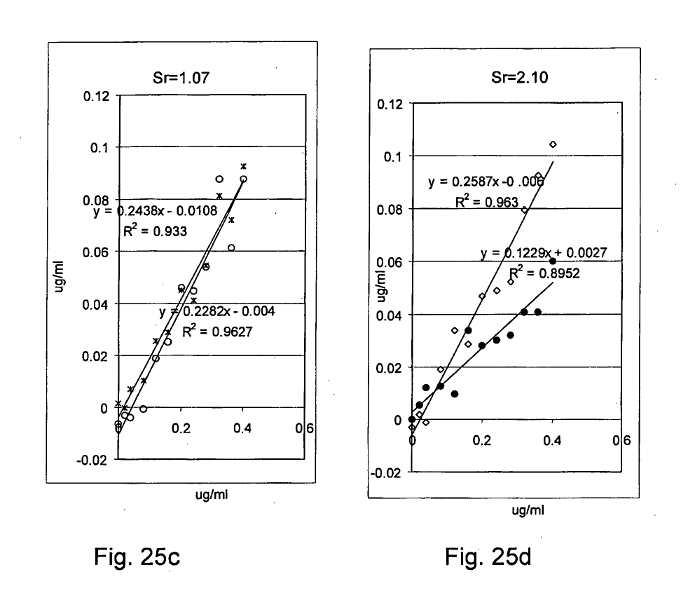

FIGS. 23a-24d show the results of the Ab* assays for the overnight incubation at 4° C. (FIGS. 23a-d) and the 2 hours incubation at 37° C. (FIGS. 24a-d) in NonSorp™ (a), medium Costar™ (b), PolySorp™ (c) and MaxiSorp™ (d) plates. Similarly to FIGS. 21a-22d, the results obtained using the liquid composition of the present invention and the control are marked with full and empty symbols, respectively. As shown in FIGS. 23a-24d, except for two occurrences (overnight incubation in the NonSorp™ plate, and 2 hours in the PolySorp™ plate), the slopes obtained using the liquid composition of the present invention are steeper than the slopes obtained in the control experiments. Specifically, the calculated values of the Sr parameter obtained for FIGS. 23a-d were, 0.94, 1.10, 1.20 and 1.27, respectively, while the calculated values of the Sr parameter obtained for FIGS. 24a-d were, 1.16, 1.35, 0.94 and 1.11, respectively.

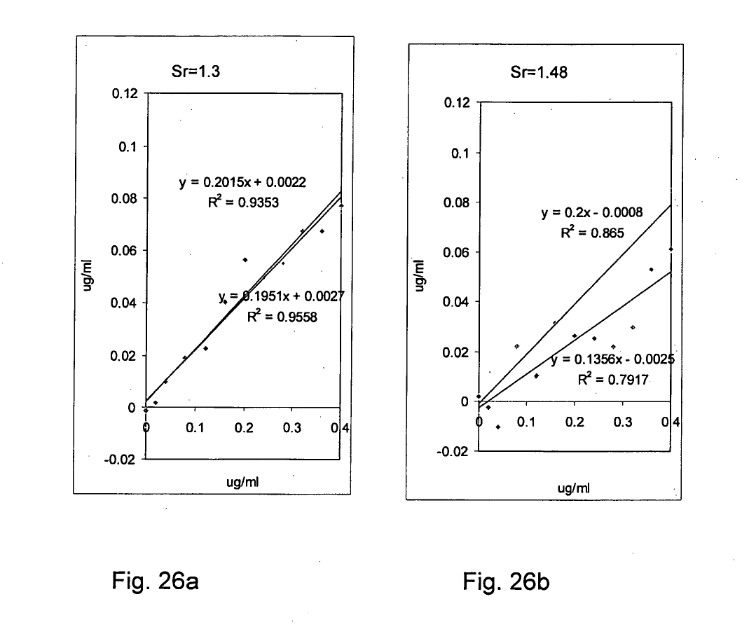

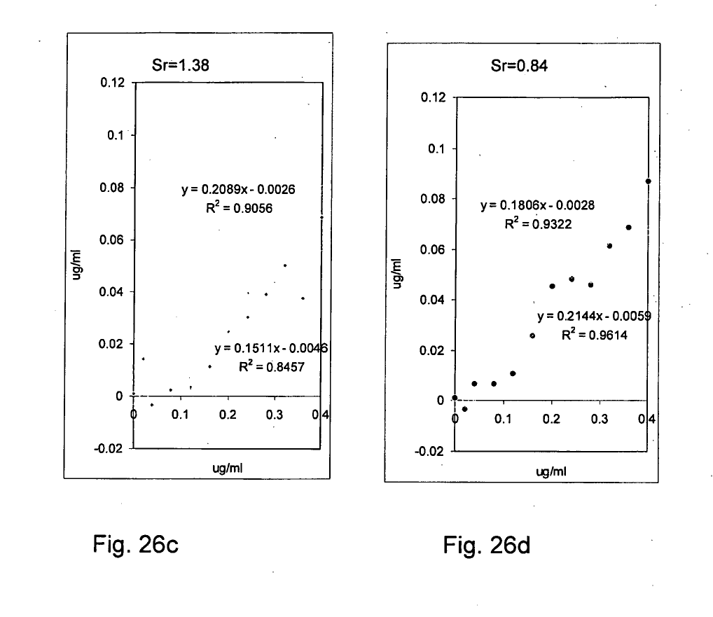

FIGS. 25a-26d show the results of the Ab*/Ab assays for the overnight incubation at 4° C. (FIGS. 25a-d) and the overnight incubation at room temperature (FIGS. 26a-d) in the medium Costar™ (a), PolySorp™ (b), MaxiSorp™ (c) and Non-Sorp™ (d) plates. As shown in FIGS. 25a-26d, except for one occurrence (incubation at room temperature in the non-sorp plate) the slopes obtained using the liquid composition of the present invention are steeper than the slopes obtained in the control. Specifically, the calculated values of the Sr parameter obtained for FIGS. 25a-d were, 1.15, 1.25, 1.07 and 2.10, respectively, and the calculated values of the Sr parameter obtained for FIGS. 26a-d were, 1.30, 1.48, 1.38 and 0.84, respectively.

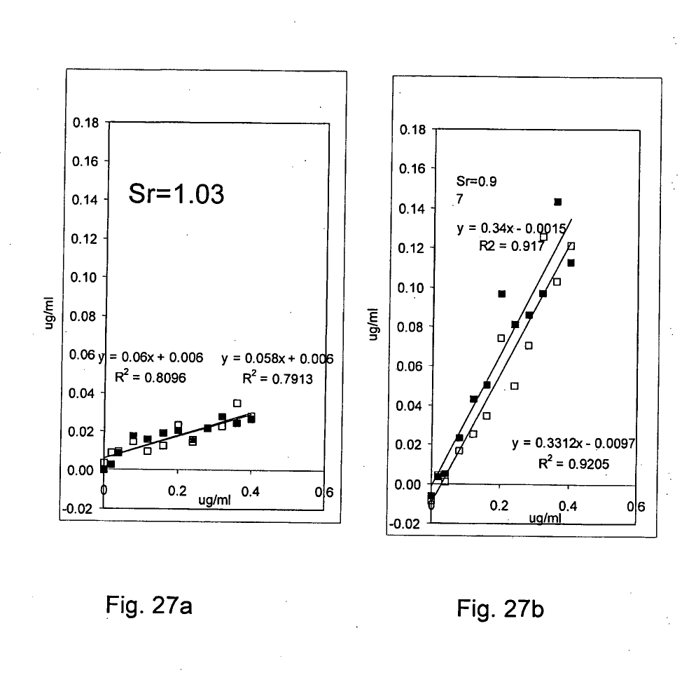

Different washing protocols are compared in FIG. 27a-dusing the medium Costar™. Hence, FIGS. 27a-b show the results of the Ab*/Ab (FIG. 27a) and Ab* (FIG. 27b) assays when phosphate buffer was used as the washing buffer, and FIGS. 27c-d show the results of Ab*/Ab (FIG. 27c) and Ab* (FIG. 27d) assays using PBS. The calculated values of the Sr parameter for the Ab*/Ab and Ab* assays (FIGS. 27a-d) were, respectively, 1.03, 0.97, 1.04 and 0.76.

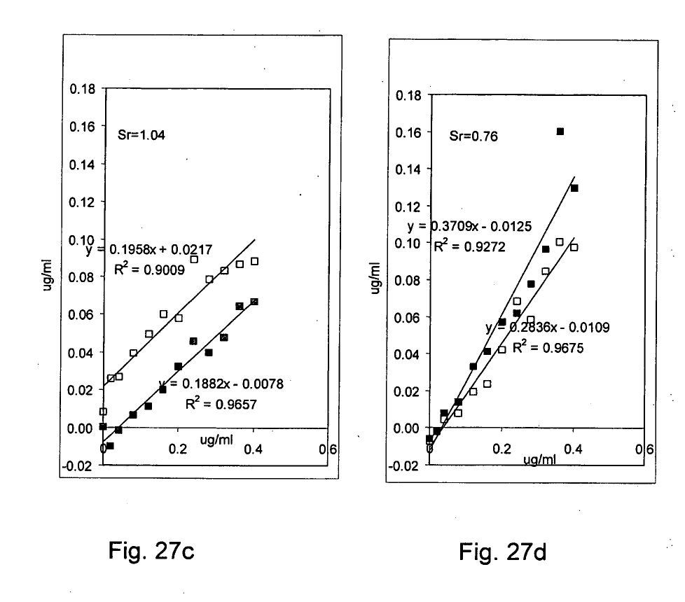

FIGS. 28a-b show the results of a single experiment in which the medium Costar™ plate was used for an overnight incubation at 4° C. (see the first experiment in Table 13). As shown in this experiment, the calculated values of the Sr parameter were 0.37 for the Ab*/Ab assay (FIG. 28a) and 0.67 for the Ab* assay (FIG. 28b).

Table 17 below, summarizes the results of FIGS. 21a-28b in terms of binding enhancement (Sr>1) and binding suppression (Sr<1) for each of the aforementioned plates.

TABLE 17

| | Medium | | | |
|---|---|---|---|---|
| Sr | costar | Polysorp | Maxisorp | Non-sorp |
| >1 | 8/12 | 5/6 | 6/6 | 4/6 |
| >1.05 | 5/12 | 5/6 | 6/6 | 4/6 |
| >1.1 | 4/12 | 5/6 | 5/6 | 3/6 |
| <1 | 4/12 | 1/6 | 0/6 | 2/6 |
| <0.95 | 3/12 | 1/6 | 0/6 | 2/6 |
| <0.9 | 3/12 | 0/6 | 0/6 | 1/6 |

As demonstrated in Table 17 and FIGS. 21a-28b, the liquid composition of the present invention enhances IgG binding, with more pronounced effect on the MaxiSorp™ and PolySorp™ plates.

Lectin Results and Discussion

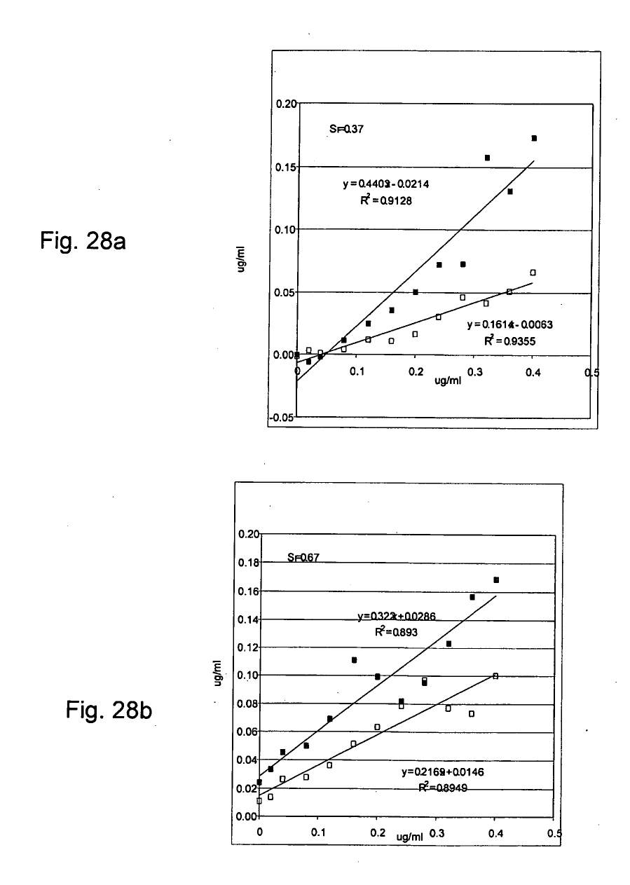

FIGS. 29a-c show the results of the PNA absorption assay to the Non-Sorp™ plate for the acetate (FIG. 29a), carbonate (FIG. 29b) and phosphate (FIG. 29c) buffers. In FIGS. 29a-c, the results obtained using the liquid composition of the present invention are marked with open symbols and results of the control are marked with full symbols.

The calculated values of the Sr parameter for the acetate, carbonate and phosphate buffers were 0.65, 0.75 and 0.78, respectively. Thus, in all three buffers the liquid composition of the present invention significantly inhibits the binding of PNA.

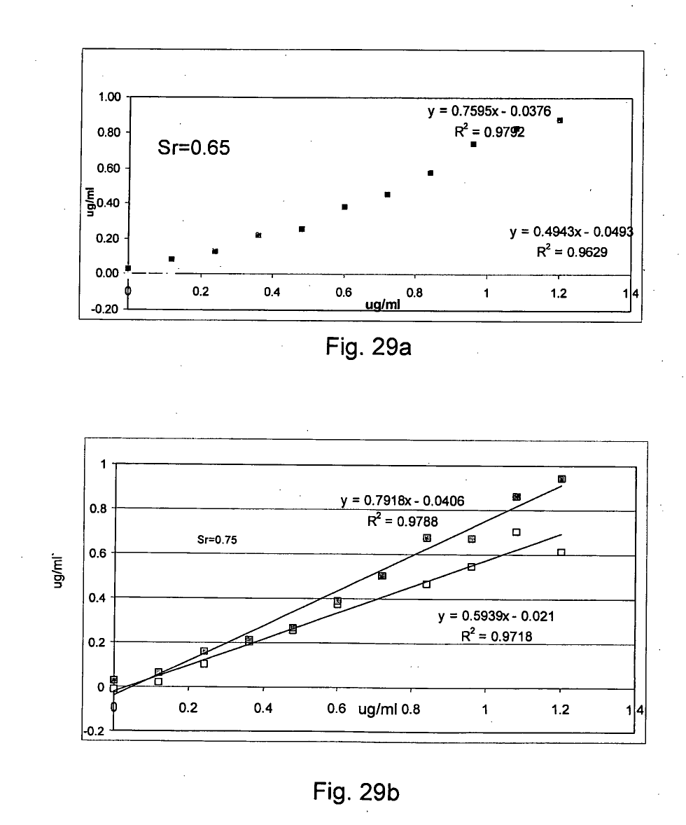

FIGS. 30a-d show the results of PNA absorption assay to the in which MaxiSorp™ plate in carbonate (FIGS. 30a-b), acetate (FIG. 30c) and phosphate (FIG. 30d) coating buffers were used. Similar symbols as in FIGS. 29a-c were used for presentation. With referring to FIG. 30a, in the carbonate buffer, a two-phase curve was obtained, having a linear part in low protein concentration in which no effect was observed and a nonlinear part in high concentration (above about 0.72) in which the liquid composition of the present invention significantly inhibits the binding of PNA. FIG. 30b presents the linear part of the graph, and a calculated value of Sr parameter of 1.01 for the carbonate buffer. The calculated values of the Sr parameter for the acetate and phosphate buffers were 0.91 and 0.83, respectively, indicating similar trend in which the liquid composition of the present invention inhibits the binding of PNA.

The results of the PNA* assay are summarized in Table 18, below, in terms of binding enhancement (Sr>1) and binding suppression (Sr<1).

TABLE 18

| Sr | MaxiSorp ™ | Non-Sorp ™ |
|---|---|---|
| >1 | 1/3** | 0/3 |
| >1.05 | 0/3 | 0/3 |
| >1.1 | 0/3 | 0/3 |
| <1 | 2/3 | 0/0 |
| <0.95 | 2/3 | 0/3 |
| <0.9 | 1/3 | 3/3 |

**Sr was calculated for the liner part of the graph.

Hence, in the Non-Sorp™ plate, the inhibition was not effected by the different buffers (pH). On the other hand, in the MaxiSorp™ plate, a pronounced effect was observed in the carbonate buffer were the curve saturated. This can be explained by dissociation of the four subunits, which effectively increases the number of competing molecules.

Note that the two proteins, IgG and PNA, behave in an opposite manner on the MaxiSorp™ plates. This indicates that the liquid composition of the present invention effect the molecular structure of the proteins.

Oligonucleotides Results and Discussion

The oligonucleotied was bound only to the MaxiSorp™ plates in acetate coating buffer.

Table 19 below summarizes the obtained values of the Sr parameter, for nine different concentrations of the oligonucleotied and four different experimental conditions, averaged over the assays in which MaxiSorp™ plates in acetate coating buffer were used.

TABLE 19

| | conditions | | | | |
|---|---|---|---|---|---|
| μg/ml | 37° C. | 4° C. | 37° C. + Na | 4° C. + Na | average |
| 0.4 | 1.32 | 1.20 | 1.75 | 2.17 | 1.61 |
| 0.36 | 1.33 | 1.44 | 1.30 | 1.17 | 1.31 |
| 0.32 | 0.98 | 1.31 | 1.17 | 1.30 | 1.19 |
| 0.28 | 1.38 | 1.47 | 1.27 | 1.34 | 1.36 |
| 0.24 | 1.16 | 1.16 | 1.13 | 1.26 | 1.18 |
| 0.20 | 1.26 | 1.23 | 0.94 | 1.09 | 1.13 |
| 0.16 | 1.08 | 1.16 | 1.22 | 1.20 | 1.16 |
| 0.12 | 0.89 | 1.18 | 1.34 | 1.57 | 1.24 |
| 0.08 | 1.21 | 1.03 | 0.93 | 1.29 | 1.11 |

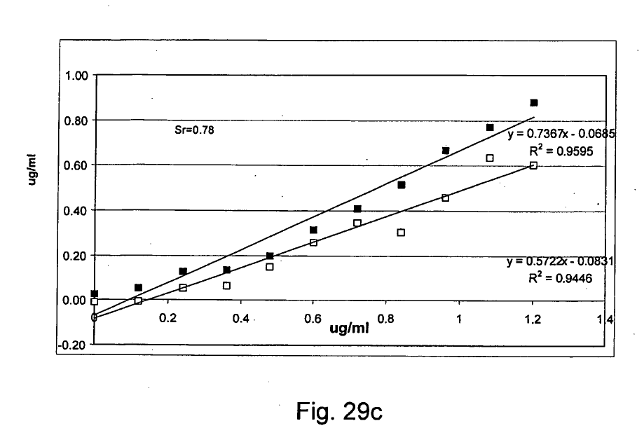

FIGS. 31a-b show the average values of the Sr parameter quoted in Table 19, where FIG. 31a show the average values for each experimental conditions and FIG. 31b show the overall average, with equal weights for all the experimental conditions.

As shown in FIGS. 31a-b, the average values of the Sr parameter were significantly larger then 1, with higher binding efficiency for higher concentration of oligonucleotied. Thus, it can be concluded the liquid composition of the present invention is capable of enhancing binding efficiency with and without the addition of salt to the coating buffer.

It is a common knowledge that acetate buffer is used to precipitate DNA in aqua's solutions. Under such conditions the DNA molecules interact to form “clamps” which are precipitated on the bottom of the plate, creating regions of high concentration on the bottom of the plate, thereby increasing the probability to bind and to generate higher signal per bounding event. Intra-molecular interactions compete with the mechanism of clamp formations. In contrast to the control water, the liquid composition of the present invention is capable of suppressing the enhancement of clamp formations for higher concentration.

The higher binding efficiency to DNA on MaxiSorp™ plates using acetate buffer composed of the liquid composition of the present invention, demonstrates the capability of the liquid composition of the present invention to at least partially de-fold DNA molecules. This feature of the present invention was also observed in DNA electrophoresis experiments, as further detailed in Example 14, below.

### Example 12

### Isolation and Purification of DNA

Nucleic acids (DNA and RNA) are the basic and most important material used by researchers in the life sciences. Gene functions, biomolecule production, drug development (pharmacogenomics) are fields that are routinely applying nucleic acids techniques. These techniques include DNA and RNA extraction, purification and amplification. DNA purification and determination is performed before and after amplification of specific regions. The amplification process is typically performed by PCR. After PCR reaction the amplified DNA is purified from oligonucleotide primers, primer dimmers, deoxinucleotide bases (A, T, C, G) and salt.

Materials and Methods:

The effect of liquid composition of the present invention on the purification processes was studied by reconstitution of the Promega kit “Wizard—PCR preps DNA purification system” (A7170).

The use of Promega Wizard™ kit involves the following steps:

1) Mix the purification buffer with the PCR sample to create conditions for binding the DNA to the Resin.

2) Mix the Resin suspension with the PCR mixture, for binding the DNA to the Resin, applies the resin samples to syringes and generate vacuum.

3) Add Isopropanol and suck the solution by vacuum to remove non bound DNA.

4) Elute the bound DNA by water.

5) Perform gel electrophoresis as further detailed hereinbelow.

Reconstitution of the kit was performed replacing aqua solutions of the kit (hereinafter control) with either RO water or the liquid composition of the present invention for each of steps 1, 2 and 4, where in step 3 the same 80% Isopropanol solution was used in all experiments.

The following protocol was used for gel electrophoresis:

(a) Gel solution: 8% PAGE (+Urea) was prepared with either RO water or the liquid composition of the present invention according to Table 20, below.

TABLE 20

| | |
|---|---|
| Total volume | 250 (ml) |
| 40% acrylamid | 50 |
| 10 x TBE | 25 |
| Urea | 84.1 g |
| RO/liquid composition | about 105 |

(b) Prepare four empty gel cassettes (Rhenium Ltd, Novex NC2015, 09-01505-C2).

(c) Add polymerization reagents containing 405 μl 10% APS and 55 μl TEMED (Sigma T-7024) to 50 ml of gel solution.

(d) Pour the gel solution to the cassette, place the plastic combs and allow polymerization for 30 minutes at room temperature.

(e) Take the combs out and strip off the tape at their bottom to allow assembling of two gels on two opposite sides of a single device.

(f) Fill in the inner chamber to the top of the gel and the outer chamber to about fifth of the gel height with running buffer-TBE×1 in either RO water or the liquid composition of the present invention.

(g) Wash.

(h) Prepare samples by diluting them in sample buffer containing TBE Ficoll, Bromophenol blue and urea (SBU), and mix 1:1 with the DNA sample.

(i) Load 8-10 μl of the mix into each well.

(j) Set the power supply to 100 V and let the migration continue until the color (Bromophenol blue) reaches 1 cm from the end.

The following protocol was used for gel staining visualization photographing and analyzing:

(a) Place the gels in staining solution containing 1 U/μl GelStar™ in 1× TBE for 15 minutes shaking.

(b) Distain the gels for 30 minutes in 1× TBE buffer.

(c) Place the gels on U.V. table; use 365 nm light to see the DNA.

(d) Using DC120™ digital camera, photograph the gels and store the digital information for further analysis.

PCR was prepared from Human DNA (Promega G 3041) using primers, specific to ApoE Gene (fragment size 265 bp), according to the following protocol (for 100 reactions):

(a) Mark 0.2 μl PCR-tubes according to the appropriate serial number.

(b) Add 2.5 μl of 40 μg/ml Human DNA (Promega G 3041) or water to the relevant tubes.

(c) Adjust to 17 μl with 14.5 μl DDW.

(d) Prepare 3630 μl of the PCR mix according to Table 21 (see below).

(e) Add 33 μl of the mix to each tube.

(f) Place the samples in the PCR.

(g) Run a PCR program according to Table 22 (see below).

(h) Analyze 5 μl of each product on 8% PAGE gel.

(i) Store reactions at −20° C.

TABLE 21
PCR Mix

| | |
|---|---|
| DDW | 836 |
| DMSO 100% | 550 |
| fw primer 1* (10 μM) | 550 |
| rv primer 2* (10 μM) | 550 |
| 10 × PCR buffer (15 mM MgCl) | 550 |
| dNTPs (2 mM) | 550 |
| MgC1 (25 mM) | 0 |
| Taq polymerase (5 μ/ul) | 44 |
| total in μl | 3630 |

*primer 1 5′TCCAAGGAGCTGCAGGCGGCGCA (SEQ ID NO: 1)
*primer 1 6-fam5′mTCCAAGGAGCTGCAGGCGGCGCA (SEQ ID NO: 2)
*primer 1 biotin5′bTCCAAGGAGCTGCAGGCGGCGCA (SEQ ID NO: 3)
*primer 2 5′GGCGCTCGCGGATGGCGCTGAG (SEQ ID NO: 4).

TABLE 22
PCR program

| | Temperature | time |
|---|---|---|
| 1 | 95° C. | 5 min |
| 2 | 94° C. | 1 min |
| 3 | 68° C. | 1 min |
| 4 | 72° C. | 30 sec |
| 5 | go back to step 2 ×34 times | |
| 6 | 72° C. | 5 min |
| 7 | 4° C. | hold |

Results:

For clarity, in the present and following Examples, control is abbreviated as “CO,” Reverse Osmosis water is abbreviated as “RO,” and the liquid composition of the present invention is abbreviated as “LC.”

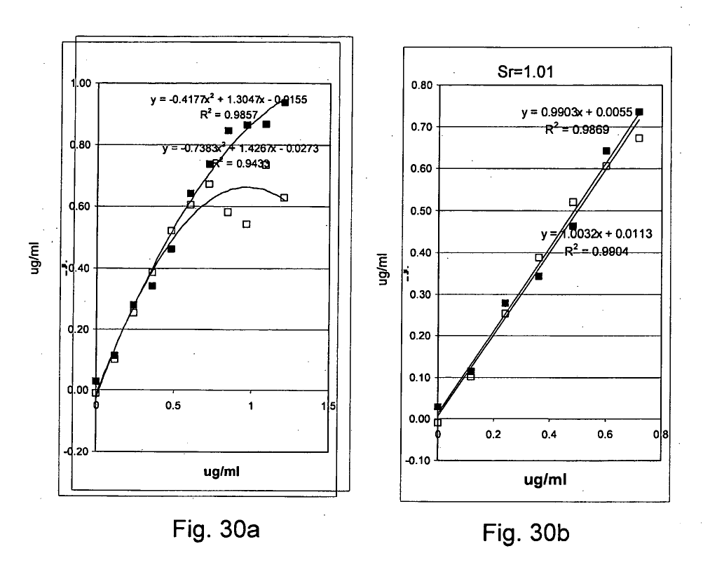

FIG. 32 is an image of 50 μl PCR product samples in an experiment, referred to herein as Experiment 3. There are 11 lanes in FIG. 32, in which lane 1 correspond to the PCR product before purification, lane 7 is a ladder marker, and lanes 2-6, 8-11 correspond the following combinations of the aforementioned steps 1, 2 and 4: CO/CO/CO (lane 2), RO/RO/RO (lane 3), LC/LC/LC (lane 4), CO/CO/CO elution 2 (lane 5), RO/RO/RO elution 2 (lane 6), LC/LC/LC elution 2 (lane 8), CO/CO/CO elution 3 (lane 9), RO/RO/RO elution 3 (lane 10), and LC/LC/LC elution 3 (lane 11).

All three assays systems exhibit similar purification features. Efficient removal of the low M.W molecules (Lower than 100 bp) is demonstrated. The removed molecules are the primers and their dimmers as well as Nucleotide bases.

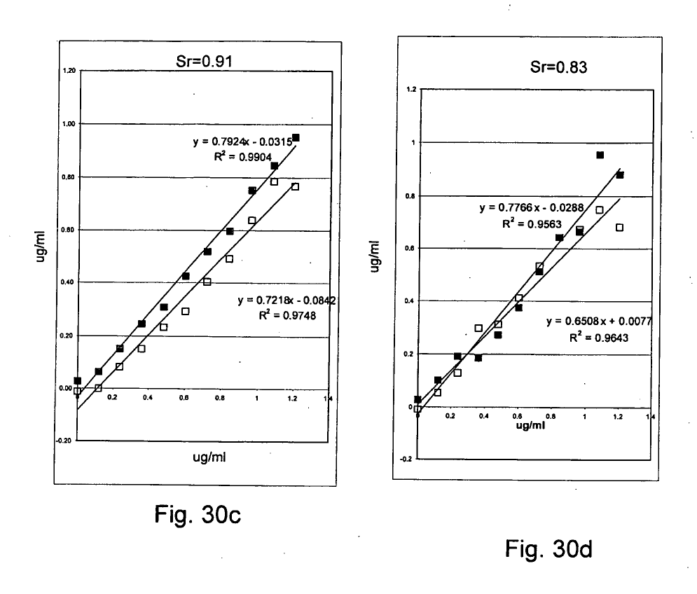

FIGS. 33a-b are images of 50 μl PCR product samples in an experiment, referred to herein as Experiment 4, for elution 1 (FIG. 33a) and elution 2 (FIG. 33b). There are 13 lanes in FIGS. 33a-b, in which lane 6 is a ladder marker, and lanes 1-5, 7-13 correspond the following combinations: CO/CO/CO (lane 1), RO/RO/RO (lane 2), LC/LC/LC (lane 3), CO/LC/LC (lane 4), CO/RO/RO (lane 5), CO/CO/LC (lane 7), CO/CO/RO (lane 8), CO/LC/CO (lane 9), CO/RO/CO (lane 10), LC/LC/CO (lane 11), RO/RO/CO (lane 12), LC/LC/LC (lane 13), where in lane 13 a different concentration was used for the liquid composition of the present invention.

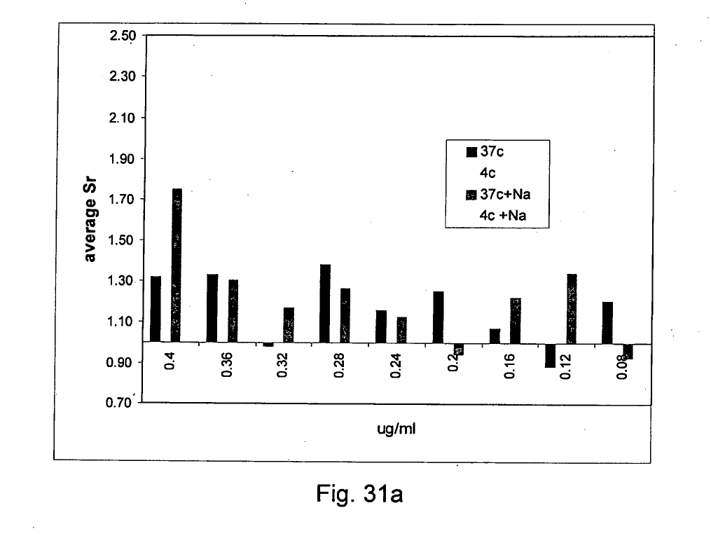

FIGS. 34a-b are images of 50 μl PCR product samples in an experiment, referred to herein as Experiment 5, for elution 1 (FIG. 34a) and elution 2 (FIG. 34b). In FIGS. 34a-b, lane 4 is a ladder marker, and lanes 1-3, 5-13 correspond the following combinations: CO/CO/CO (lane 1), RO/RO/RO (lane 2), LC/LC/LC (lane 3), CO/LC/LC (lane 5), CO/RO/RO (lane 6), CO/CO/LC (lane 7), CO/CO/RO (lane 8), CO/LC/CO (lane 9), CO/RO/CO (lane 10), LC/LC/CO (lane 11), RO/RO/CO (lane 12), and LC/CO/CO (lane 13). Lane 14 in FIG. 34a corresponds to the combination RO/CO/CO.

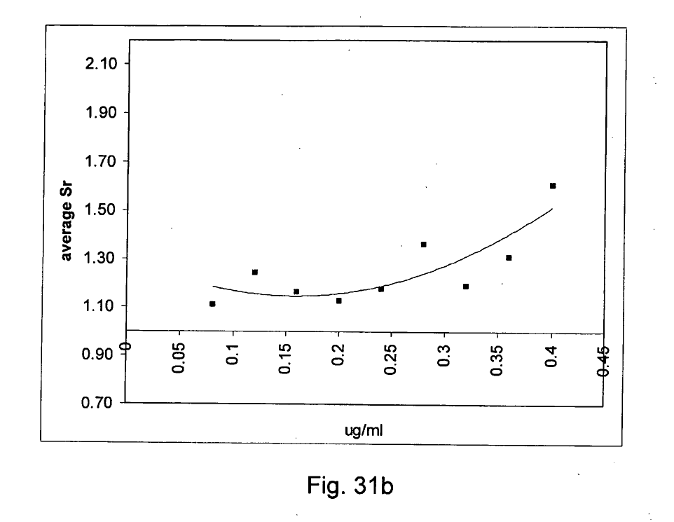

FIGS. 35a-b are images of 50 μl PCR product samples in an experiment, referred to herein as Experiment 6, for elution 1 (FIG. 35a) and elution 2 (FIG. 35b). In FIGS. 35a-b, lanes 1-13 correspond to the same combinations as in FIG. 34a, and lane 15 corresponds to the PCR product before purification.

### Example 13

### Column Capacity

In this example, the effect of the liquid composition of the present invention on column capacity was examined. 100 PCR reactions, each prepared according to the protocols of Example 12 were produced and combined to 5 ml stock solution. The experiment, referred to herein as Experiment 7, included two steps, in which in a preliminary step (hereinafter step A) was directed at examining the effect of volume applied to the columns on binding and elution, and a primary step (hereinafter step B) was directed at investigating the effect of the liquid composition of the present invention on the column capacity.

In Step A, four columns (columns 1-4) were applied with 50, 150, 300 or 600 μl stock PCR product solution, and 13 columns (5-17) were applied with 300 μl of stock PCR solution. All columns were eluted with 50 μl water. The eluted solutions were applied to lanes 7-10 in the following order: lane 7 (original PCR, concentration factor×1), lane 8 (original×3), lane 9 (×6) and lane 10 (×12). An additional lane, lane 11, was applied with a “mixture” of all elution of columns 5-17 (×6). Lanes 1-5 were applied with elution from columns 1-4 and the “mixture” of columns 5-17, pre-diluted to the original concentration (×1). Lane 6 was the ladder marker.

The following protocol was employed in Step A:

1) Mark the Wizard™ minicolumn and the syringe for each sample, and insert into the Vacuum Manifold.

2) Dispense 100 μl of each direct PCR purification buffer solution into a micro-tube.

3) Vortex briefly.

4) Add 1 ml of each resin solution and vortex briefly 3 times for 1 minute.

5) Add the Resin/DNA mix to the syringe and apply vacuum.

6) Wash by adding 2 ml of 80% isopropanol solution to each syringe and apply vacuum.

5) Dry the resin by maintaining the vacuum for 30 seconds.

6) Transfer the minicolumn to 1.5 ml microcentrifuge tube.

7) Centrifuge at 10000 g for 2 minutes.

8) Transfer the minicolumn to clean 1.5 ml tube.

9) Add 50 μl of the relevant water (nuclease free or the liquid composition of the present invention).

10) Centrifuge at 10000 g for 20 second.

11) Transfer 50 μl storage microtube and store at −20° C.

12) Repeat steps 9-11 for a second elution cycle.

Visualization steps:

13) Mix 6 μl of each sample with 6 μl loading buffer.

14) Load 10 μl of each mix in acrylamide urea gel (AAU) and run the gel at 70 V 10 mAmp.

15) Stain the gel with Gel Star™ solution (5 μl of 10000 u solution in 50 ml TBE), shake for 15 minutes at room temperature.

16) Shake in TBE buffer at room temperature for 30 minutes to distain the gel.

17) Image the gel.

In Step B the “mixture” elution of Step A was used as “concentrated PCR solution” and applied to 12 columns. Columns 1-5 were applied by 8.3 μl, 25 μl, 50 μl, 75 μl and 100 μl using the kit reagents. The columns were eluted by 50 μl kit water and 5 μl of each elution was applied to the corresponding lane on the gel. Columns 7-11 were treated as column 1-5 but with the liquid composition of the present invention as binding and elution buffers. The samples were applied to the corresponding gel lanes. Column 13 served as control with the “mixture” of columns 5-17 of Step A.

The following protocol was employed in Step B:

1) Mark the Wizard™ minicolumn and syringe to be used for each sample and insert into the vacuum manifold.

2) Dispense 100 μl of each direct PCR purification buffer solution into micro-tube.

3) Vortex briefly.

4) Add 1 ml of each resin solution and vortex briefly 3 times for 1 minutes.

5) Add the Resin/DNA mix to the syringe and apply vacuum.

6) Wash by adding 2 ml of 80% isopropanol solution to each syringe and apply vacuum.

5) Dry the resin by continuing to apply the vacuum for 30 seconds.

6) Transfer the minicolumn to 1.5 ml microcentrifuge tube.

7) Centrifuge at 10000 g for 2 minutes.

8) Transfer the minicolumn to clean 1.5 ml tube.

9) Add 50 μl of nuclease free or the liquid composition of the present invention.

10) Centrifuge at 10000 g for 20 seconds.

11) Transfer 50 μl storage micro-tube and store at −20° C.

12) Repeat steps 9-11 for a second elution cycle.

Visualization steps were the same as in Step A.

Results:

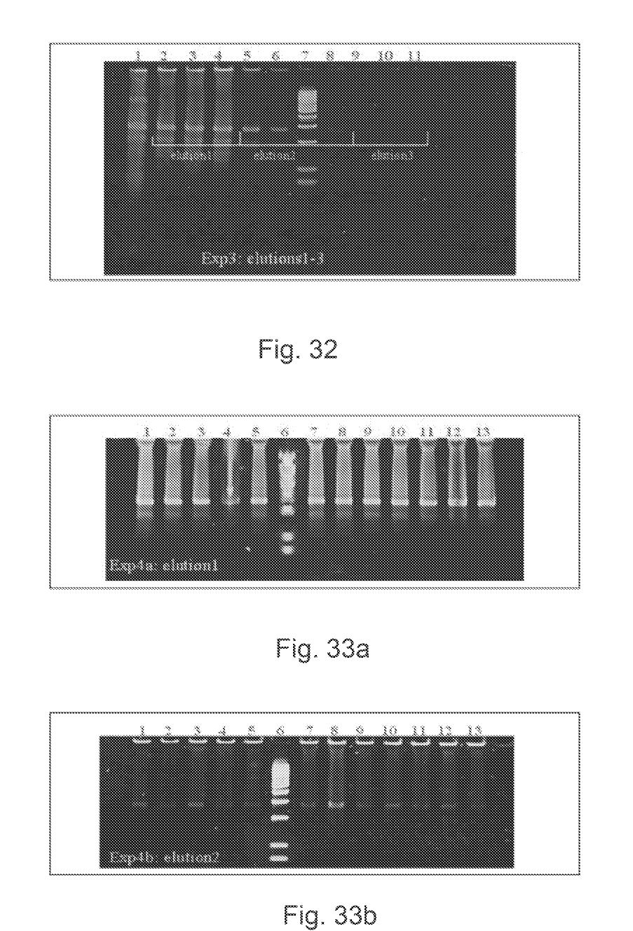

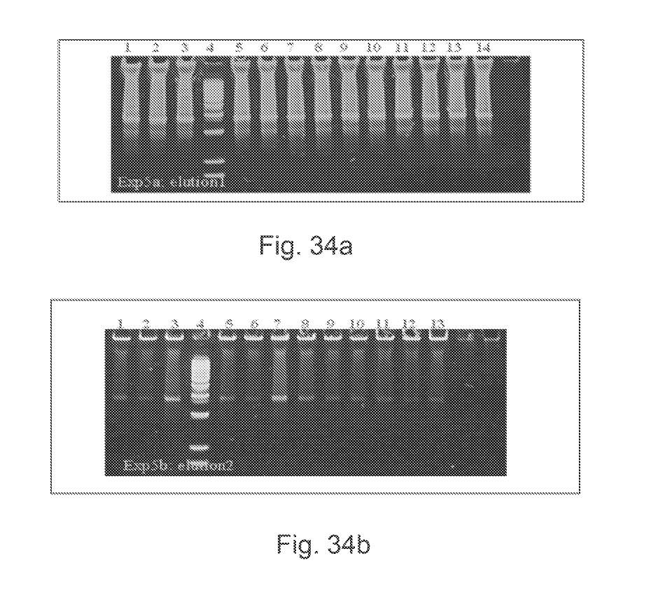

FIGS. 36-37 show image (FIG. 36) and quantitative analysis using Sionlmage™ software (FIG. 37) of lanes 1-11 of Step A. As shown in FIG. 36a, lanes 8-11 are overloaded. Lane 3 and 4 contain less DNA because columns 3, 4 were overloaded and as a result less DNA were recovered after dilution of the eluted samples.

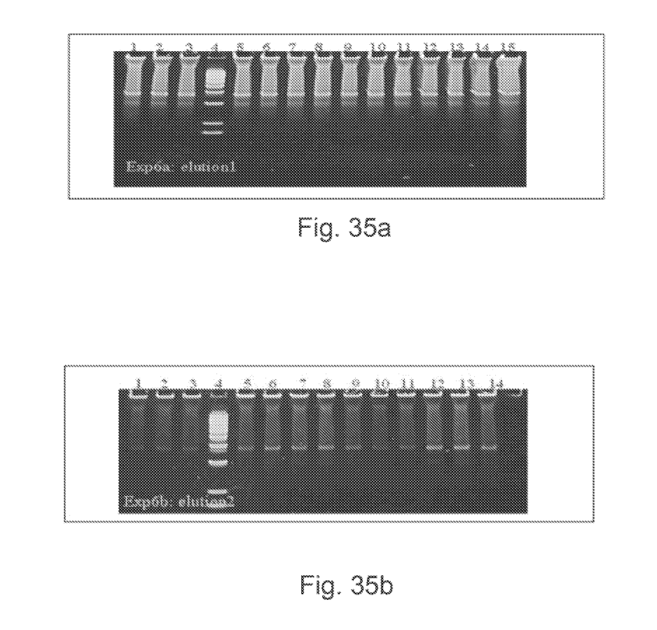

FIGS. 38a-c shows image of lanes 1-12 of Step B, for elution 1 (FIG. 38a), elution 2 (FIG. 38b) and elution 3 (FIG. 38c). The first elution exhibits similar picture as of overloading, and the differences in binding capacity is clearly seen in the second elution. The band intensity increases with the ascending number of the lane.

Comparing the intensity of corresponding lanes 1-5 and 7-11 indicates that the liquid composition of the present invention is capable of bounding more DNA then the kit reagents.

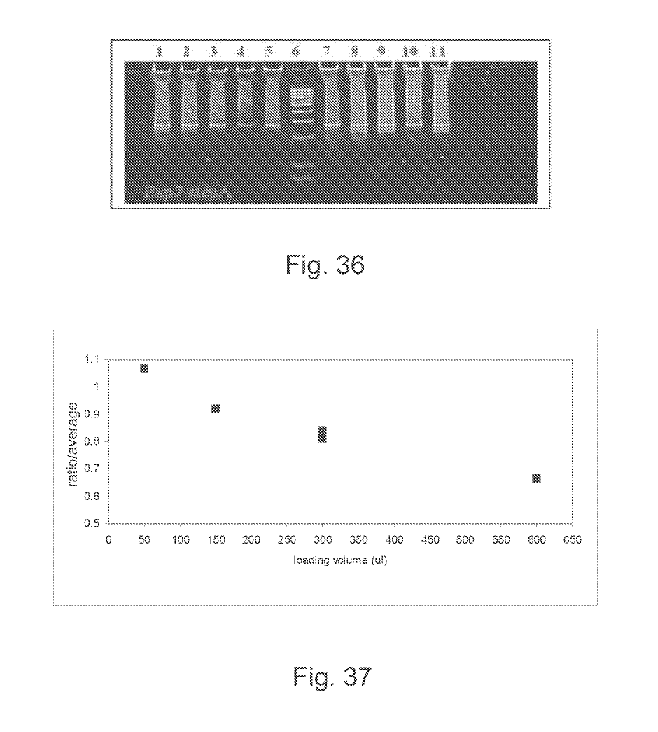

FIGS. 39a-b show quantitative analysis using SionImage™ software, where FIG. 39a presents the area of the control (designated CO in FIGS. 39a-b) and the liquid composition of the present invention (designated LC in FIGS. 39a-b) as a function of the loading volume for each of the three elutions, and FIG. 39b shows the ratio LC/CO. As shown in FIGS. 39a-b in elution 3, the area is larger for the liquid composition of the present invention.

### Example 14

### Isolation of DNA by Gel Electrophoresis

Gel Electrophoresis is a routinely used method for determination and isolation of DNA molecules based on size and shape. DNA samples are applied to an upper part of the gel, serving as a running buffer surrounding the DNA molecules. The gel is positively charged and forces the negatively charged DNA fragments to move downstream the gel when electric current is applied. The migration rate is faster for smaller and coiled or folded molecules and slower for large and unfolded molecules. Once the migration is completed, DNA is tagged by fluorescent label and is visualized under UV illumination. The DNA can be also transferred to a membrane and visualized by enzymatic coloration at high sensitivity. DNA is evaluated according to its position on the gel and the band intensity.

Following is a description of experiments in which the effect of the liquid composition of the present invention on DNA migration by gel electrophoresis was examined.

Materials and Methods:

Two types of DNA were used: (i) PCR product, 280 base pair; and (ii) ladder DNA composed of eleven DNA fragments of the following sizes: 80, 100, 200, 300, 400, 500, 600, 700, 800, 900 and 1030 bp. The gel was prepared according to the protocols of Example 12.

Three experiments were performed. In Experiment 1, PCR batch number 181103 was fed into lanes 2-10 with the ladder DNA in lane 1; in Experiment 2, PCR batch number 31203 was fed into lanes 2-11 with the ladder DNA in lane 1; and in Experiment 3, PCR batch number 31203 was fed into lanes 1-5 and 7-11, with the ladder DNA in lane 6.

Results:

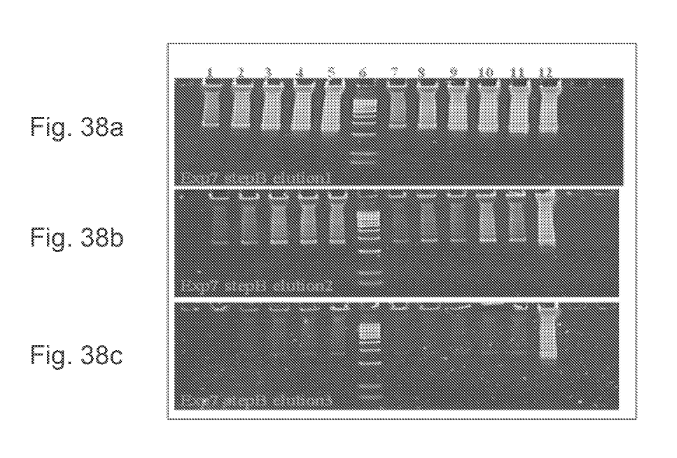

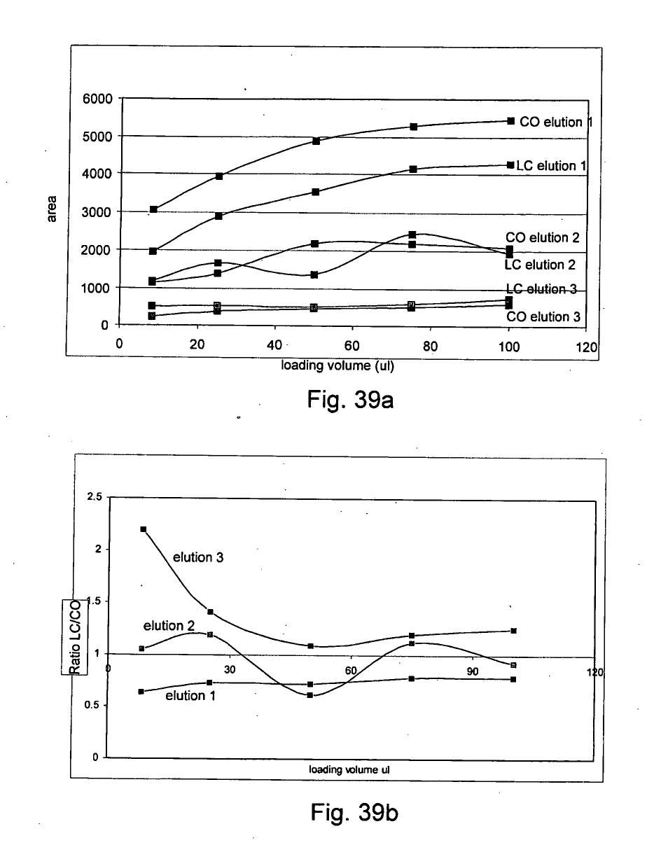

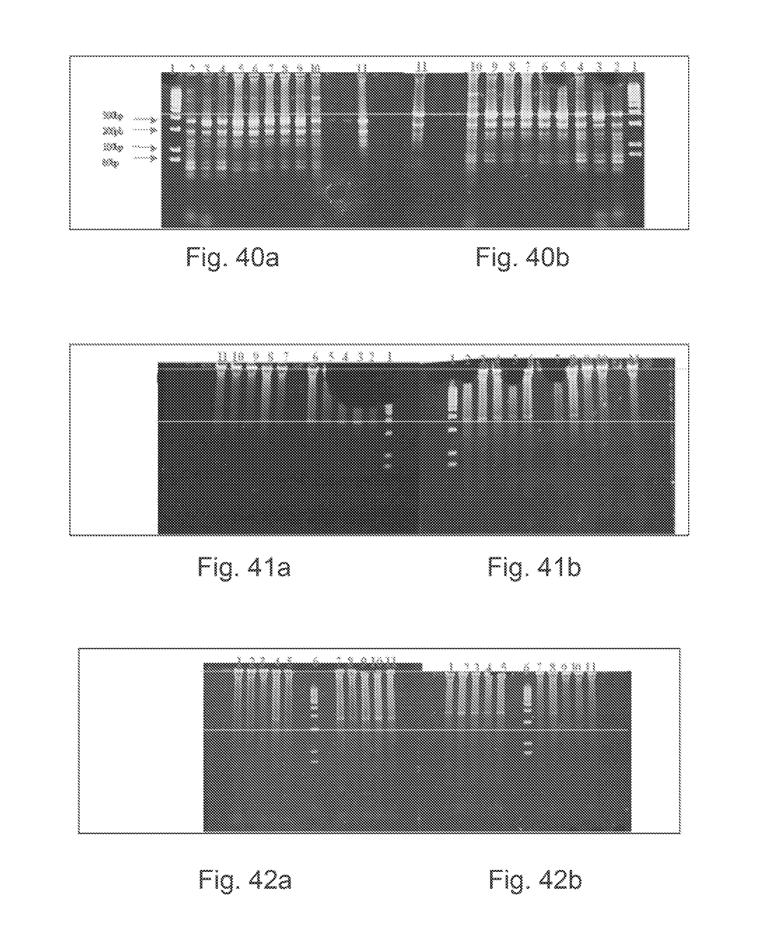

FIGS. 40a-42b are lane images comparing the migration speed in the presence of RO water (FIGS. 40a, 41a and 42a) and in the presence of the liquid composition of the present invention (FIGS. 40b, 41b and 42b) for Experiments 1, 2 and 3, respectively. In the images of FIGS. 40a-42b both the running buffers and the gel buffers were composed of the same type of liquid, i.e., in FIGS. 40a, 41a and 42a both the running buffer and the gel buffer were composed of RO water, while in FIGS. 40b, 41b and 42b both the running buffer and the gel buffer were composed of the liquid composition of the present invention.

As shown in FIGS. 40a-42b, both type of DNA (PCR product and the ladder DNA) were migrating significantly faster in RO water in comparison to the liquid composition of the present invention.

In trying to separate the effect of the liquid composition of the present invention on the gel content and its effect on the running buffer, the above experiments were repeated in all possible combinations of running and gel buffers.

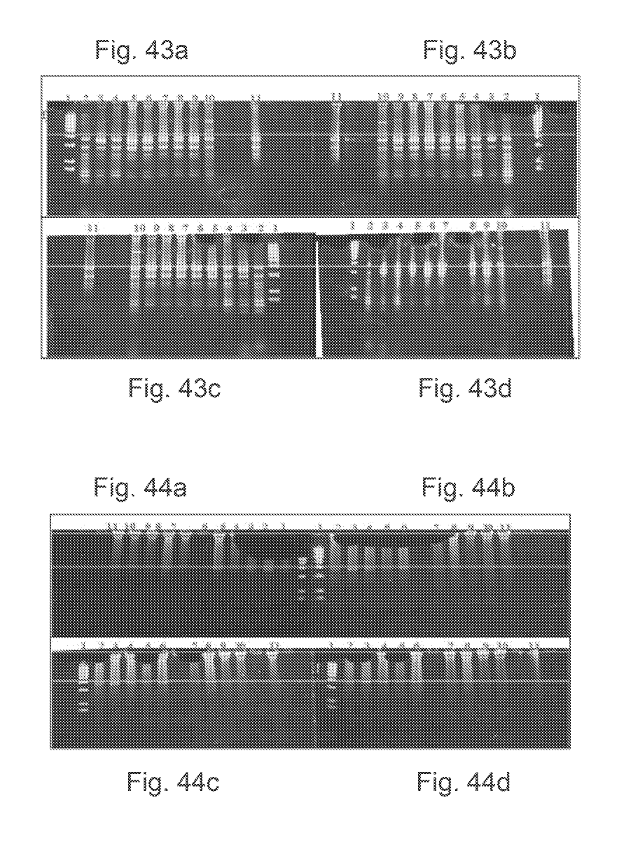

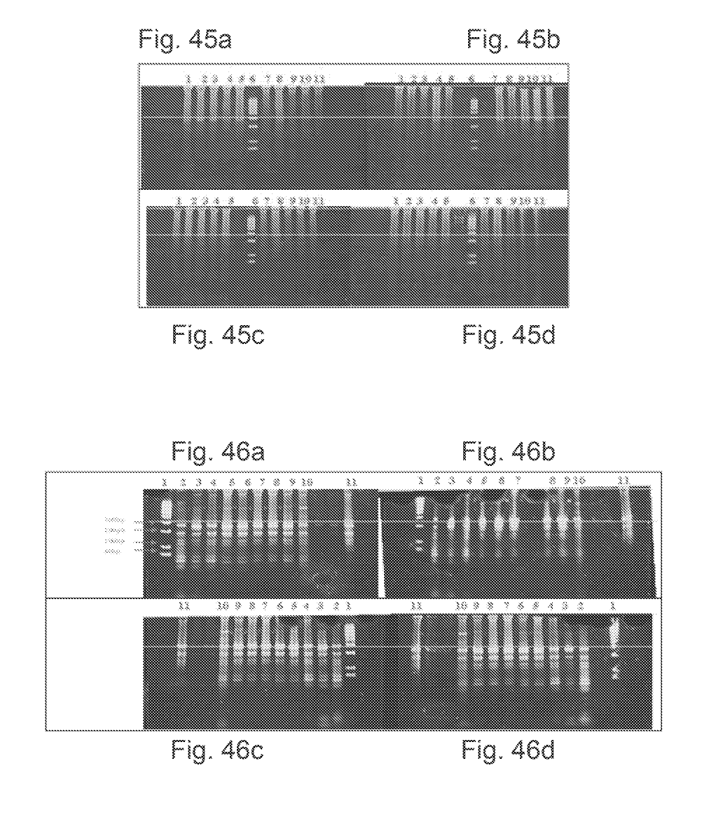

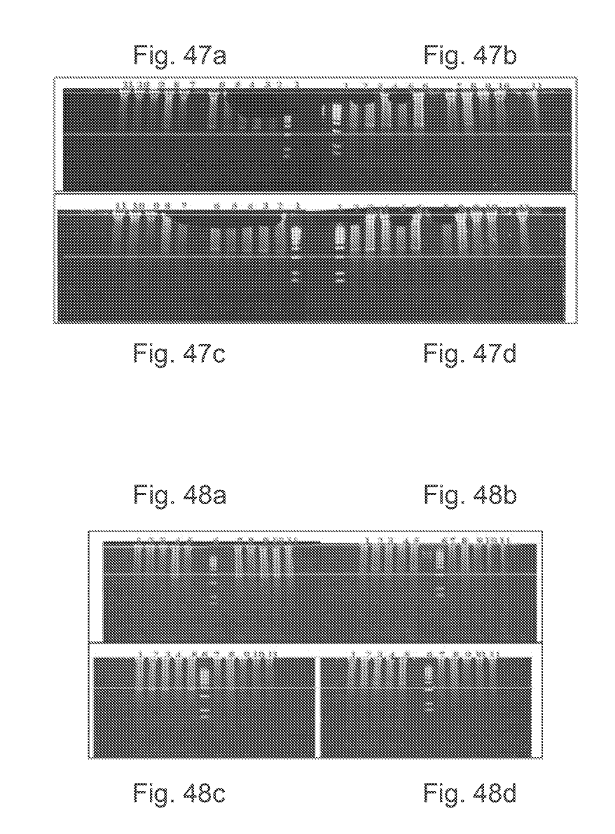

Hence, FIGS. 43a-45d are lane images of Experiments 1 (FIGS. 43a-d), 2 (FIGS. 44a-d) and 3 (FIGS. 45a-d), in which the effect of the liquid composition of the present invention on the running buffer are investigated. In each pair of figures (i.e., pairs a-b and c-d) the gels are composed of the same liquid and the running buffer is different. Using the abbreviations introduced in Example 12, the following combinations of gel/running buffers are shown in FIGS. 43a-45d: FIGS. 43a-b are images of RO/RO and RO/LC, respectively; FIGS. 43c-d are images of LC/LC and LC/RO respectively, FIGS. 44a-b are images of RO/RO and RO/LC, respectively; FIGS. 44c-d are images of LC/RO and LC/LC respectively, FIGS. 45a-b are images of RO/LC and RO/RO, respectively; and FIGS. 45c-d are images of LC/LC and LC/RO respectively.

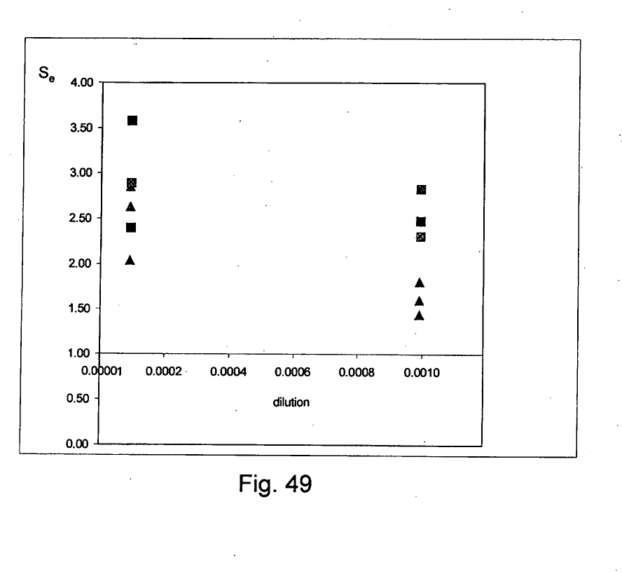

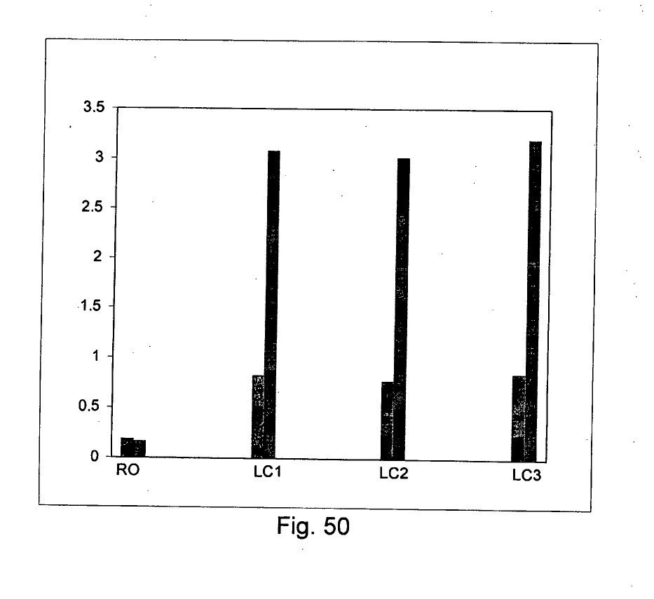

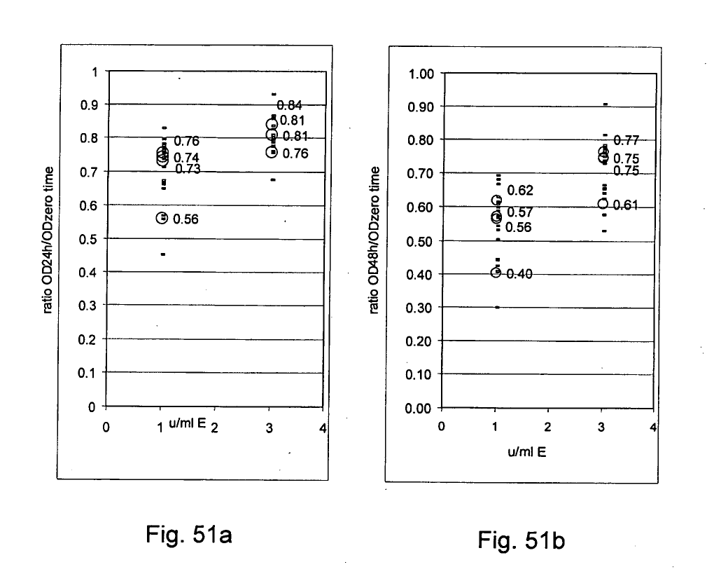

FIGS. 46a-48d are lane images of Experiments 1 (FIGS. 46a-d), 2 (FIGS. 47a-d) and 3 (FIGS. 48a-d), in which the effect of the liquid composition of the present invention on the gel buffer are investigated. In each pair of figures (a-b, c-d) the running buffers are composed of the same liquid and the gel buffer are different. Specifically, FIGS. 46a-b are images of RO/RO and LC/RO, respectively; FIGS. 46c-d are images of LC/LC and RO/LC respectively, FIGS. 47a-b are images of RO/RO and LC/RO, respectively; FIGS. 47c-d are images of RO/LC and LC/LC respectively, FIGS. 48a-b are images of RO/RO and LC/RO, respectively; and FIGS. 48c-d are images of RO/LC and LC/LC respectively.

As shown in FIGS. 43a-48d, in the presence of the liquid composition of the present invention, the migration of the DNA molecule is slower than in the presence of RO water. Note that no significant change in the electric field was observed. This effect is more pronounced when the gel buffer is composed of the liquid composition of the present invention, while the running buffer is composed of RO water.

Thus, the above experiments demonstrate that under the influence of the liquid composition of the present invention, the DNA configuration is changed, in a manner that the folding of the DNA is decreased (un-folding). The un-folding of DNA in the liquid composition of the present invention may indicate that stronger hydrogen boned interactions exists between the DNA molecule and the liquid composition of the present invention in comparison to RO water.

### Example 15

### Enzyme Activity and Stability

Improvement of Enzyme activity and stability are important for increasing efficiency and reducing costs of any process utilizing Enzymes. During storage or prolong activity, enzymes are typically exposed to stress which may contribute to lose of activity. The activity and stability throughout time is a highly important factor for the user and for the producer of the enzyme.

In this example, the effect of the liquid composition of the present invention on activity and satiability of enzymes is demonstrated. This study has covered two enzymes, which are commonly used in the biotechnological industry: Alkaline Phosphatase (AP), and β-Galactosidase. Two forms of AP were used: an unbound form and a bound form in which AP was bound to Strept-Avidin (ST-AP).

Following is a description of experiments in which the effect of the liquid composition of the present invention on diluted enzymes was investigated. The dilutions were performed either in RO water or in the liquid composition of the present invention without additives and in natural pH (7.4).

### Unbound Form of Alkaline Phosphatase

Materials and Methods:

Alkaline Phosphatase (Jackson INC) was serially diluted in either RO water or the liquid composition of the present invention. Diluted samples 1:1,000 and 1:10,000 were incubated in tubes at room temperature.

At different time intervals, enzyme activity was determined by mixing 10 μl enzyme with 90 μl pNPP solution (AP specific colorimetric substrate). The assay was performed in microtitration plates (at least 4 repeats for each test point). Color intensity was determined by an ELISA reader at wavelength of 405 nm.

Enzyme activity was determined at time t=0 for each dilution, both in RO water and in three different concentrations of the liquid composition of the present invention: LC3, LC7 and LC8 as further detailed hereinabove. Stability was determined as the activity after 22 hours and 48 hours divided by the activity at t=0.

Results & Discussion:

Tables 23-25 below summarize the average activity values of six experiments, enumerated 1-6, for t=0 (Table 23), t=22 hours (Table 24) and t=48 hours (Table 25). Experiments 1-5 were conducted at room temperature and Experiment 6 was conducted at 4° C.

TABLE 23

| | | average activity | | | | | |
|---|---|---|---|---|---|---|---|
| liquid | dilution | 1 | 2 | 3 | 4 | 5 | 6 |
| RO | 1:1000 | 3.27 | 2.91 | 2.72 | 1.74 | 2.46 | 2.89 |
| | 1:10000 | 0.49 | 0.35 | 0.37 | 0.29 | 0.45 | 0.42 |
| | 0 | 0.07 | 0.08 | 0.08 | 0.10 | 0.08 | 0.08 |
| LC7 | 1:1000 | 3.55 | 3.51 | 3.39 | 3.39 | 0.08 | 3.43 |
| | 1:10000 | 0.62 | 0.56 | 0.55 | 0.63 | 0.08 | 0.58 |
| | 0 | 0.08 | 0.08 | 0.08 | 0.08 | 0.08 | 0.08 |
| LC8 | 1:1000 | 3.44 | 3.34 | 3.45 | 3.54 | 3.37 | 3.55 |
| | 1:10000 | 0.58 | 0.45 | 0.56 | 0.58 | 0.48 | 0.59 |
| | 0 | 0.08 | 0.08 | 0.08 | 0.09 | 0.08 | 0.08 |
| LC3 | 1:1000 | 3.47 | 3.39 | 3.44 | 3.60 | 2.87 | 3.48 |
| | 1:10000 | 0.63 | 0.68 | 0.80 | 0.67 | 0.41 | 0.55 |
| | 0 | 0.08 | 0.08 | 0.08 | 0.08 | 0.09 | 0.08 |

TABLE 24

| | | average activity | | | | | |
|---|---|---|---|---|---|---|---|
| liquid | dilution | 1 | 2 | 3 | 4 | 5 | 6 |
| RO | 1:1000 | 1.78 | 0.77 | 2.01 | 0.36 | 1.46 | 1.70 |
| | 1:10000 | 0.28 | 0.14 | 0.17 | 0.17 | 0.19 | 0.22 |
| | 0 | 0.04 | 0.07 | 0.07 | 0.06 | 0.04 | 0.08 |
| LC7 | 1:1000 | 3.42 | 2.47 | 3.10 | 2.64 | | 2.57 |
| | 1:10000 | 0.45 | 0.32 | 0.40 | 0.40 | | 0.47 |
| | 0 | 0.04 | 0.08 | 0.09 | 0.06 | | 0.08 |
| LC8 | 1:1000 | 3.27 | 2.23 | 3.42 | 3.39 | 3.02 | 3.30 |
| | 1:10000 | 0.45 | 0.27 | 0.08 | 0.47 | 0.47 | 0.47 |
| | 0 | 0.04 | 0.04 | 0.05 | 0.04 | 0.04 | 0.08 |
| LC3 | 1:1000 | 3.50 | 3.31 | 3.36 | 3.15 | 3.08 | 3.31 |
| | 1:10000 | 0.56 | 0.55 | 0.61 | 0.58 | 0.46 | 0.48 |
| | 0 | 0.08 | 0.04 | 0.08 | 0.08 | 0.05 | 0.08 |

TABLE 25

| | | average activity | | | | | |
|---|---|---|---|---|---|---|---|
| liquid | dilution | 1 | 2 | 3 | 4 | 5 | 6 |
| RO | 1:1000 | 1.34 | 0.49 | 0.86 | 0.22 | 0.60 | 1.34 |
| | 1:10000 | 0.22 | 0.12 | 0.11 | 0.13 | 0.13 | 0.22 |
| | 0 | 0.08 | 0.08 | 0.08 | 0.08 | 0.08 | 0.08 |
| LC7 | 1:1000 | 3.03 | 2.43 | 2.05 | 2.16 | | 3.03 |
| | 1:10000 | 0.37 | 0.31 | 0.23 | 0.29 | | 0.37 |
| | 0 | 0.08 | 0.08 | 0.08 | 0.08 | | 0.08 |
| LC8 | 1:1000 | 2.48 | 2.32 | 2.07 | 2.67 | 1.78 | 2.48 |
| | 1:10000 | 0.37 | 0.25 | 0.27 | 0.41 | 0.26 | 0.37 |
| | 0 | 0.05 | 0.07 | 0.07 | 0.08 | 0.08 | 0.05 |
| LC3 | 1:1000 | 3.27 | 3.57 | 2.22 | 2.58 | 1.83 | 3.27 |
| | 1:10000 | 0.46 | 0.45 | 0.42 | 0.45 | 0.31 | 0.46 |
| | 0 | 0.08 | 0.08 | 0.07 | 0.08 | 0.08 | 0.08 |

As shown in Tables 23-25 the activity in the presence of LC7, LC8 and LC3 is consistently above the activity in the presence of RO water. To quantify the effect of the liquid composition of the present invention on the stability, a stability enhancement parameter, Sₑ, was defined as the stability in the presence of the liquid composition of the present invention divided by the stability in RO water.

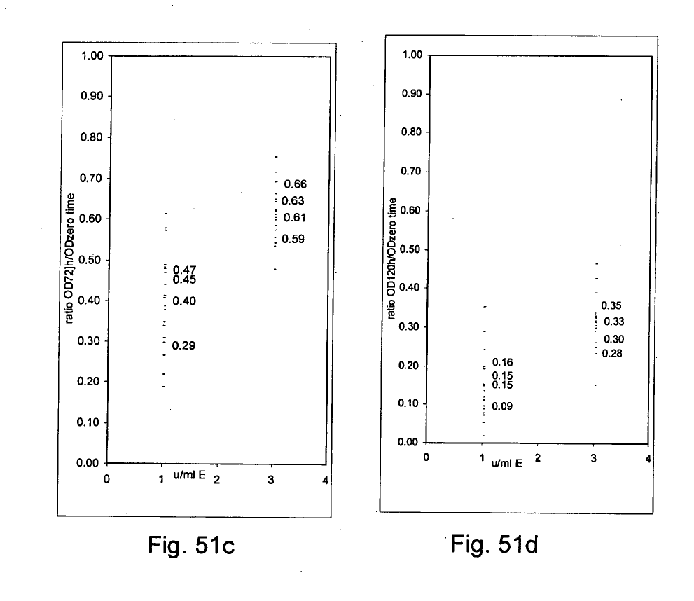

FIG. 49 shows the values of Sₑ, for 22 hours (full triangles) and 48 hours (full squares), as a function of the dilution. The values of Sₑ for LC7, LC8 and LC3 are shown in FIG. 49 in blue, red, and green, respectively). As shown in FIG. 49, the measured stabilizing effect is in the range of about 2 to 3.6 for enzyme dilution of 1:10,000, and in the range of about 1.5 to 3 for dilution of 1:1,000. The same phenomena were observed at low temperatures, although in somewhat to a less extent.

### Bound Form of Alkaline Phosphatase

Binding an enzyme to another molecule typically increases its stability. Enzymes are typically stored in high concentration, whereby prior to their use the enzymes are diluted to the desired dilution. The following experiments are directed at investigating the stabilization effect of the liquid composition of the present invention when the enzymes are in high concentration for a prolonged period of time.

Materials and Methods:

Strept-Avidin Alkaline Phosphatase (Sigma) was diluted 1:10 and 1:10,000 in RO water and in the aforementioned liquid compositions LC7, LC8 and LC3 of the present invention. The diluted samples were incubated in tubes for 5 days at room temperature.

All samples were diluted to final enzyme concentrated of 1:10,000 and the activity was determined as further detailed hereinabove. Enzyme activity was determined at time t=0 and after 5 days.

Results and Discussion:

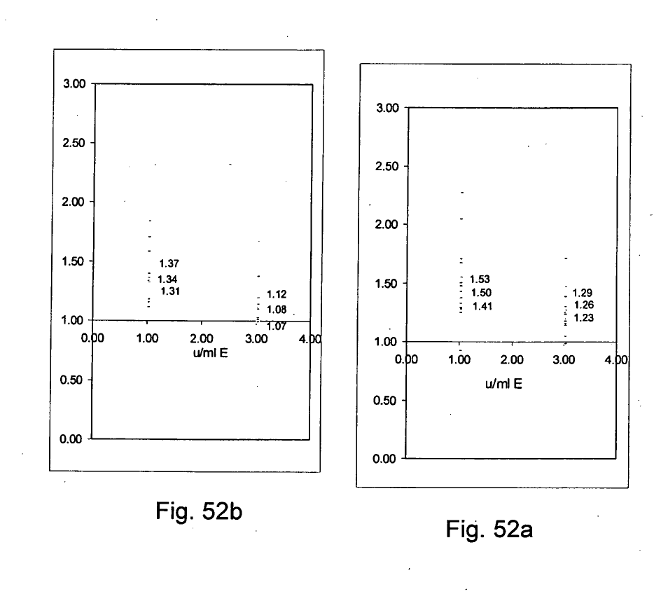

FIG. 50 is a chart showing the activity of the conjugated enzyme after 5 days of storage in a dilution of 1:10 (blue) and in a dilution of 1:10,000 (red), for the RO water and the liquid composition of the present invention. In RO water the enzyme activity is about 0.150 OD for both dilutions. In contrast, in the presence of the liquid composition of the present invention the activity is about 3.5 times higher in the 1:10 dilution substantially without any activity lost during 5 days of storage.

### β-Galactosidase

Materials and Methods:

The experiments with β-Galactosidase were performed according to the same procedure which was employed in the Alkalin Phosphatse experiments described above. The modifications of the present experiments were only in enzyme type and concentration and in incubation time. Hence, β-Galactosidase (Sigma) was serially diluted in RO water and in the liquid composition of the present invention. The samples were diluted to 1:330 and 1:1000 and were incubated at room temperature.

At different time intervals, namely, 0, 24 hours, 48 hours, 72 hours and 120 hours, the enzyme activity was determined by mixing 10 μl enzyme with 100 μl ONPG solution (β-Gal specific colorimetric substrate) for 15 minutes at 37° C. and adding 50 μl stop solution (1M Na₂Hco₃). The assay was performed in microtitration plates (8 repetitions from each test point). An ELISA reader at wavelength of 405 nm was used to determine color intensity.

The enzyme activity was determined at time t=0 for each dilution, for the RO water and for the aforementioned liquid compositions LC7, LC8 and LC3 of the present invention. Five experiments were performed under identical conditions. The enzyme stability and the stability enhancement parameter, Sₑ, were calculated as further detailed hereinabove.

Results and Discussion:

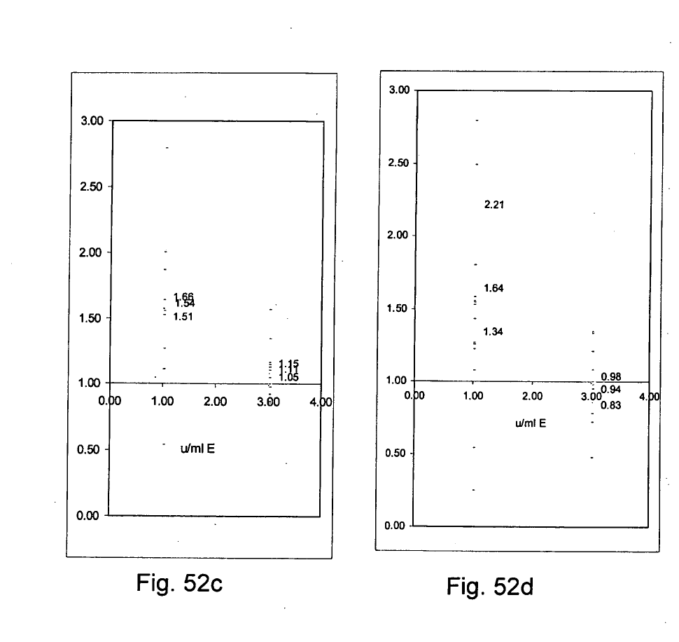

FIGS. 51a-d show the stability (the activity at time t≠0, divided by the activity at t=0), at t=24 hours (FIG. 51a), t=48 hours (FIG. 51b), t=72 hours (FIG. 51c) and t=120 hours (FIG. 51d). The liquids RO, LC7, LC8, LC3 and LC4 are shown in FIGS. 51a-d in blue, red, green and purple, respectively, and average values of the stability are shown as circles. As shown in FIGS. 51a-51d, the activity in the presence of LC7, LC8 and LC3 is consistently above the activity in the presence of RO water.

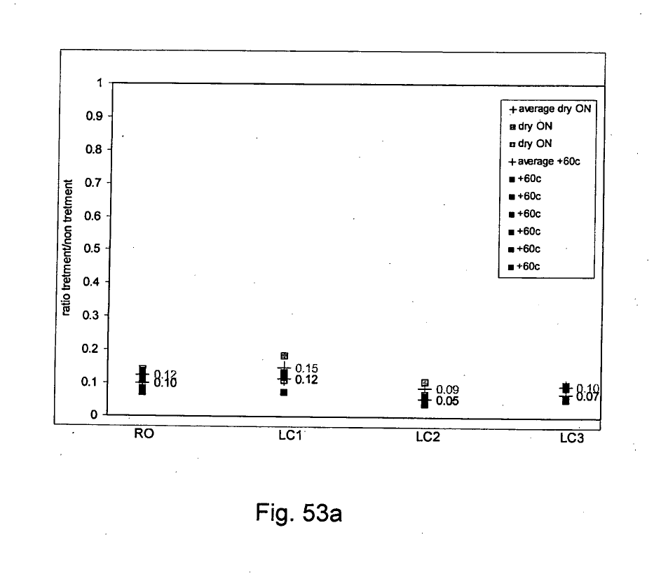

FIGS. 52a-d show the stability enhancement parameter, Sₑ, at t=24 hours (FIG. 52a), t=48 hours (FIG. 52b), t=72 hours (FIG. 52c) and t=120 hours (FIG. 52d), with similar color notations as in FIGS. 51a-d. As shown in FIG. 52a-d, the measured stabilizing effect is in the range of about 1.3 to 2.21 for enzyme dilution of 1:1000, and in the range of about 0.83 to 1.3 for dilution of 1:330.

Thus, the stabilizing effect liquid composition of the present invention on β-Galactosidase is similar to the stabilizing effect found for AP. The extent of stabilization is somewhat lower. This can be explained by the relatively low specific activity (464 u/mg) having high protein concentration in the assay, which has attenuated activity lost over time.

### Activity and Stability of Dry Alkaline Phosphatase

Many Enzymes are dried before storage. The drying process and the storage at dry state for a prolonged period of time are known to damage the enzymes. Following is a description of experiments directed at investigation the effect of the liquid composition of the present invention on the activity and stability of dry alkaline phosphatase.

Materials and Methods:

Alkaline Phosphatase (Jackson INC) was diluted 1:5000 in RO water and in the in the aforementioned liquid compositions LC7, LC8 and LC3 of the present invention, as further detailed hereinabove.

Nine microtitration plates were filled with aliquot of 5 μl solution. One plate was tested for enzyme activity in time t=0, as further detailed hereinabove, and the remaining 8 plates were dried at 37° C. overnight. The drying process was performed under desiccated environment for 16 hours.

Two plates were tested for enzyme activity by cooling the plates to room temperature and adding 100 μl pNPP solution in room temperature. Color intensity was determined by an ELISA reader at wavelength of 405 nm and the stability was calculated as further detailed hereinabove. Six plates were transferred to 60° C. for 30 minutes and the enzyme activity was determined thereafter.

Results:

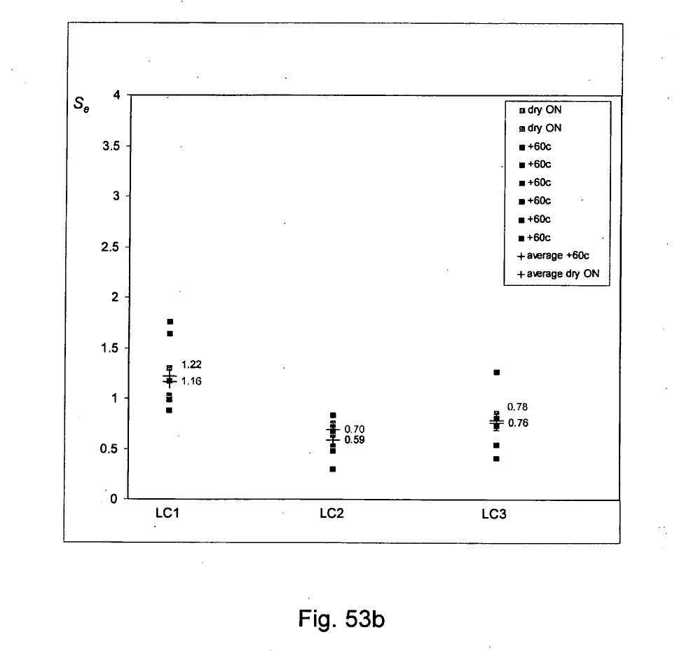

FIG. 53a shows the remaining activity of the enzymes after drying (two repeats) and after 30 minutes of heat treatment at 60° C. (6 repeats). Average values are shown in FIG. 53a by a “+” symbol. Both treatments substantially damaged the enzyme and their effect was additive.

FIG. 53b shows the stability enhancement parameter, Sₑ. In spite of the relatively small database and the extreme conditions to which the enzyme was exposed, the liquid composition of the present invention has evidently stabilized the activity of the enzyme. For example, for LC7 the average value of the stability enhancement parameter was increased from 1.16 to 1.22.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of producing a liquid composition from a solid powder, the method comprising:
(a) heating the solid powder, thereby providing a heated solid powder;
(b) immersing said heated solid powder in a cold liquid; and
(c) substantially contemporaneously with said step (b), irradiating said cold liquid and said heated solid powder by electromagnetic radiation, said electromagnetic radiation being characterized by a frequency selected such that nanostructures are formed from particles of the solid powder.

2. The method of claim 1, wherein the solid powder comprises micro-sized particles.

3. The method of claim 2, wherein said micro-sized particles are crystalline particles.

4. The method of claim 3, wherein said nanostructures are crystalline nanostructures.

5. The method of claim 1, wherein said liquid comprises water.

6. The method of claim 1, wherein said water is at a temperature which is below a density anomaly temperature of said water.

7. The method of claim 1, wherein said electromagnetic radiation is a radiofrequency radiation.

8. The method of claim 1, wherein said electromagnetic radiation is continues wave a radiofrequency radiation.

9. The method of claim 1, wherein said electromagnetic radiation is a modulated radiofrequency radiation.

10. The method of claim 1, wherein the solid powder is selected from the group consisting of a ferroelectric material and a ferromagnetic material.

11. The method of claim 1, wherein the solid powder is selected from the group consisting of BaTiO₃, WO₃ and Ba₂F₉O₁₂.

12. The method of claim 1, wherein the solid powder comprises a material selected from the group consisting of a mineral, a ceramic material, glass, metal and synthetic polymer.

13. The method of claim 1, wherein said electromagnetic radiation is in the radiofrequency range.

14. The method of claim 13, wherein said electromagnetic radiation is continues wave electromagnetic radiation.

15. The method of claim 13, wherein said electromagnetic radiation is modulated electromagnetic radiation.

16. The method of claim 13, wherein an amount of heated solid powder which is immersed in said cold liquid is selected such that a concentration of said nanostructures is lower than 10²⁰ nanostructures per litter.

17. The method of claim 13, wherein an amount of heated solid powder which is immersed in said cold liquid is selected such that a concentration of said nanostructures is lower than 10¹⁵ nanostructures per litter.

18. The method of claim 1, further comprising, subsequently to (c), mixing said nanostructures with a liquid being different from said cold liquid.

19. A nanostructure, produced by the method of claim 1.

20. A composition, comprising liquid and nanostructure and being produced by the method of claim 1.


SEQUENCE LISTING (4 sequences)

SEQ ID NO: 1 - DNA, 23 bp, Artificial sequence
  Single strand DNA oligonucleotide
tccaaggagctgcaggcggcgca

SEQ ID NO: 2 - DNA, 23 bp, Artificial sequence
  Single strand DNA oligonucleotide
tccaaggagctgcaggcggcgca

SEQ ID NO: 3 - DNA, 23 bp, Artificial sequence
  Single strand DNA oligonucleotide
tccaaggagctgcaggcggcgca

SEQ ID NO: 4 - DNA, 22 bp, Artificial sequence
  Single strand DNA oligonucleotide
ggcgctcgcggatggcgctgag